(19) United States
(12) Patent Application Publication
(10) Pub. No.: US 2024/0382531 A1
(43) Pub. Date: Nov. 21, 2024

(54) CELL POPULATIONS AND GENE EXPRESSION ASSOCIATED WITH IN VITRO BETA CELL DIFFERENTIATION

(71) Applicant: President and Fellows of Harvard College, Cambridge, MA (US)

(72) Inventors: Douglas A. Melton, Cambridge, MA (US); Adrian Veres, Cambridge, MA (US); Aubrey L. Faust, Cambridge, MA (US); Henry L. Bushnell, Cambridge, MA (US)

(21) Appl. No.: 18/620,696

(22) Filed: Mar. 28, 2024

Related U.S. Application Data
(63) Continuation of application No. PCT/US22/45111, filed on Sep. 28, 2022.
(60) Provisional application No. 63/249,546, filed on Sep. 28, 2021.

(51) Int. Cl. A61K 35/39; C12N 5/071; C12N 9/22; C12N 15/11

(52) U.S. Cl. CPC A61K 35/39; C12N 5/0613; C12N 9/22; C12N 15/111; C12N 2310/20; C12N 2510/00

(57) ABSTRACT

Disclosed herein are methods for directing the differentiation of stem cells towards a specific cell type.

### CROSS-REFERENCE TO RELATED APPLICATIONS

This application is a continuation of International Application No. PCT/US2022/045111, filed on Sep. 28, 2022, which claims the benefit of U.S. Provisional Application No. 63/249,546, filed on Sep. 28, 2021. The entire teachings of the above applications are incorporated herein by reference in their entirety. International Application No. PCT/US2022/045111 was published under PCT Article 21(2) in English.

### BACKGROUND OF THE INVENTION

Pancreatic beta cells are regulators of blood glucose whose autoimmune destruction or dysfunction cause Type 1 and Type 2 diabetes. Recently, in vitro differentiation protocols have been developed that convert pluripotent stem cells into pancreatic beta cells. A challenge in producing any cell type in vitro is heterogeneity of the cells generated by directed differentiation. At each step of the process, some cells follow the desired path, others stray. To improve yield of a desired cell type, it would be beneficial to direct the differentiation process such that the cells follow a specific path resulting in the desired cell type.

### SUMMARY OF THE INVENTION

Single-cell RNA sequencing can be used to identify the various cell types produced during in vitro differentiation of pluripotent stem cells to a target cell type. The sequencing analysis can provide a view of the sequence of transcriptional changes that underlie the formation of a target cell and reveals fate-determinative decision points and alternative pathways that cells may follow along the differentiation road.

Work described herein identifies markers for cell types produced by in vitro pancreatic cell differentiation. Using this data, genes can be identified that are specifically enriched in single populations or combinations of populations. The knowledge that these genes are specific to a given population can be leveraged to further develop in vitro pancreatic beta cell differentiation methods, as well as methods for in vitro differentiation to other cell types (such as alpha cells).

More specifically, these genes can be used as targets for genetic perturbation (such as knock-out, activation or inhibition) allowing for the creation of ‘tailored’ (non-wild type, non-native) stem cell lines that have been genetically edited to be incapable of mis-differentiating toward undesired fates. That is, by controlling the gene expression of cells during the differentiation process (at the pluripotent stage and/or at one or more later points in the differentiation process), one may open or close routes of differentiation, encouraging or forcing a cell down a desired path or away from an undesired path.

Described herein are methods for directing differentiation of a population of cells comprising inhibiting expression of a regulator of cell fate in a progenitor cell, wherein the regulator is selected from Table 1 or Table 2, thereby directing differentiation of a population of cells towards SC-P cells.

In some embodiments, the regulator is selected from the group consisting of FBXL14, BCORL1, SHOC2, CCDC6, B3GALT6, HOXA1, DDX3X, CARM1, EXT2, EXT1, DYRK1A, SCAF1, SCAF8, CAND1, NDST1, EYA3, GLCE, DYRK1B, PRDM16, ALG3, CXXC4, SMURF1, PHF21A, SOX4, and TET2. In certain embodiments, the regulator is selected from the group consisting of FBXL14, BCORL1, SHOC2, CCDC6, B3GALT6, HOXA1, DDX3X, CARM1, EXT2, and EXT1. In some embodiments, the regulator is selected from the group consisting of SOX4, BCORL1, FBXL14, CCDC6, SOX1, CARM1, TNRC18, CAND1, TET2, HOXA1, ASCL1, ARID2, SIRT6, FBXO22, FLVCR1, FOXA1, COPS9, ELAVL1, SSBP3, PROSER1, PROX1, SMURF1, SCAF1, HELLS, and DACH1. In certain embodiments, the regulator is selected from the group consisting of SOX4, BCORL1, FBXL14, CCDC6, SOX1, CARM1, TNRC18, CAND1, TET2, and HOXA1.

Described herein are methods for directing differentiation of a population of cells comprising inhibiting expression of a regulator of cell fate in a progenitor cell, wherein the regulator is selected from Table 3 or Table 4, thereby directing differentiation of a population of cells towards SC-a cells.

In some embodiments, the regulator is selected from the group consisting of PDX1, CCDC6, HES1, PHF21A, PAX4, DYRK1B, DYRK1A, BCORL1, TET2, DDX3X, PROSER1, PBX1, HELLS, CAND1, EYA3, MYT1, AFF4, FBXL14, HOXA1, ZC3H15, SCAF8, PRDM16, HEXIM1, TTC14, ZRANB1, and B3GALT6. In certain embodiments, the regulator is selected from the group consisting of PDX1, CCDC6, HES1, PHF21A, PAX4, DYRK1B, DYRK1A, BCORL1, TET2, and DDX3X. In some embodiments, the regulator is selected from the group consisting of PAX4, HES1, CCDC6, SOX4, ZBTB10, PHF21A, PBX1, ARID2, TET2, BCORL1, TTC14, CAND1, PROSER1, SOX1, FBXO22, HELLS, DYRK1B, ZRANB1, DYRK1A, ASCL1, ZC3H15, SETBP1, FAM58A, MYT1, and RALGAPB. In certain embodiments, the regulator is selected from the group consisting of PAX4, HES1, CCDC6, SOX4, ZBTB10, PHF21A, PBX1, ARID2, TET2, and BCORL1.

In some embodiments, the expression of the regulator of cell fate is inhibited by knocking down or knocking out the regulator using a gene editing technique, e.g., using CRISPR. In some embodiments, the gene editing technique, e.g., CRISPR, is delivered to the progenitor cell via a retrovirus, e.g., lentivirus.

Also described herein are enriched populations of SC-0 cells produced by the methods described herein. In some embodiments, the methods produce a cell population comprising 70%, or in some aspects, 80% SC-0 cells.

Also described herein are SC-islets comprising the enriched population of SC-β cells described herein.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 provides a schematic of the genetic screening of in vitro human development. HUES8, human embryonic stem cells are grown and seeded for differentiation. The cells are differentiated until Stage 6, day 15+, stained for intracellular markers, sorted for target populations (SC-beta cells, SC-alpha cells, SC-EC cells, and Triple Negative cells) and sequenced. All tissue culture done with 100 million cells or more.

FIG. 2 shows a sorting strategy and provides a definition of cell types (SC-beta cells, SC-alpha cells, SC-EC cells, and Triple Negative cells). Cells are sorted based on their expression or lack thereof of INS, GCG, and SLC18A1.

FIG. 3 demonstrates that the technical controls in a validation experiment show expected patterns of depletion in a desired population. Each vertical plot compares a pair of population (between SC-beta, SC-alpha, and Triple Negative). Each dot represents a gene within the secondary screen and shows, on the y-axis, the effect of disrupting that gene on the likelihood of creating each cell type. Vertical bar indicates the value for the labelled control genes.

FIG. 4 shows perturbation targets (Table 1) that increase SC-beta cell differentiation relative to triple negative cell (TN). Orange dots show where perturbation effects the 50 genes in Table 1. The highlighted panel shows how this group was defined.

FIG. 5 shows perturbation targets (Table 2) that increase SC-beta cell differentiation relative to SC-EC cells. Orange dots show where perturbation effects the 50 genes in Table 2. The highlighted panel shows how this group was defined.

FIG. 6 shows perturbation targets (Table 3) that increase SC-alpha cell differentiation relative to triple negative cells (TN). Orange dots show where perturbation effects of the 50 genes in Table 3. The highlighted panel shows how this group was defined.

FIG. 7 shows perturbation targets (Table 4) that increase SC-alpha cell differentiation relative to SC-EC cells. Orange dots show where perturbation effects of the 50 genes in Table 4. The highlighted panel shows how this group was defined.

FIG. 8 shows the effects of knocking out FBXL14 using CRISPR via a lentivirus. The columns show the effect of the knock out where the orange line is the extent to which the cell population containing the knock out makes more cells of one type versus another. The three cell types measured are beta cells (SC-beta), alpha cells (SC-alpha), and enterochromaffin cells (SC-EC). TN, at the bottom of each column, refers to “triple negative” meaning the cells are not alpha, beta, nor EC. All black dots represent other gene knock outs. The same data is presented in another way in the circle, where the orange x represents the position of the knockout. This shows that knocking out FBXL14 results in the generation of more beta cells. The histograms provide quantification using different guide RNAs (gi, g2, etc.) to achieve CRISPR knockouts. Knocking out FBXL14 is shown to increase the % of beta cells (without any significant effect on EC cells).

FIG. 9 shows the effects of knocking out FBXO22 using CRISPR via a lentivirus. The columns show the effect of the knock out where the orange line is the extent to which the cell population containing the knock out makes more cells of one type versus another. The three cell types measured are beta cells (SC-beta), alpha cells (SC-alpha), and enterochromaffin cells (SC-EC). TN, at the bottom of each column, refers to “triple negative” meaning the cells are not alpha, beta, nor EC. All black dots represent other gene knock outs. The same data is presented in another way in the circle, where the orange x represents the position of the knockout. This shows that knocking out FBXO22 results in the production of fewer EC cells. The histograms provide quantification using different guide RNAs (gi, g2, etc.) to achieve CRISPR knockout. Knocking out FBXO22 is shown to decrease the % of EC cells (without any significant effect on beta cells).

FIGS. 10A-10G demonstrate single cell RNA sequencing of in vitro beta cell differentiation. FIG. 10A provides a summary of the cell populations identified by flow cytometry at the end of Stages 3-6 of the Pagliuca et al. SC-beta protocol. PDX1: pancreatic transcription factor, NKX6.1: beta cell transcription factor, INS: insulin, beta cell hormone, CHGA: chromogranin A, pan endocrine marker. FIG. 10B demonstrates using inDrops to sample cells from several time points of the same differentiation. FIG. 10C provides expression profiles of developmentally relevant genes and markers across cell types identified during SC-beta differentiation. Shading displays mean expression (z-normalized tpm) and diameter denotes fractional expression. FIGS. 10D-10G shows tSNE projections of cells sampled from the ends of Stages 3-6 of the ‘x1’ protocol. Cells are colored according to their assigned cluster. Horizontal bars indicate cell type proportions.

FIGS. 11A-11I demonstrate SC-beta cells maintain identity and gain maturation marker expression during extended culture in Stage 6. FIG. 11A provides an experimental design to study functional and transcriptional changes during Stage 6 of protocol v8. FIG. 11B shows glucose stimulated insulin secretion showing consecutive low glucose (2.8 mM) and high glucose (20 mM) challenges for three independent differentiations over a period of 5 weeks. FIG. 11C provides stimulation indices (insulin released at 20 mM glucose/insulin released at 2 mM) for data in FIG. 11B. FIG. 11D shows tSNE projection of 38,494 cells from 6 time points spanning 5 weeks of Stage 6. Cells are colored according to their assigned type. Vertical bars show population ratios in each week. FIG. 11E shows expression of endocrine marker genes. FIG. 11F shows correlation of expression profiles for each major cell type, broken down by week. Cell type colors match those in (d). FIG. 11G provides pseudotime order of SC-beta cells shown on tSNE (top) and distribution of SC-beta pseudotime order stratified by sampling week (bottom). FIG. 11H provides identification of dynamic genes along SC-beta pseudotime. Fold-change compares start and end of pseudotime trajectory. q-values are FDR adjusted (alpha=0.001) p-values from likelihood ratio test comparing full and reduced models (see methods). FIG. 11I provides expression of selected genes shown along SC-beta pseudotime. Each dot represents expression of a cell, sorted and shaded as in (FIG. 11G). Line shows result of pseudotime regression.

FIGS. 12A-12E provide characterization of stem cell derived-enterochromaffin cells (SC-EC cells). FIG. 12A provides a comparison of SC-beta and SC-EC gene expression profiles. Blue genes are required for serotonin synthesis or enterochromaffin markers. FIG. 12B shows expression levels for SC-EC enriched genes across in vitro populations (top panel) and human pancreatic endocrine cells (bottom panel). FIGS. 12C-12D show immunofluorescence staining for SC-EC cell markers showing co-localization with serotonin (5-HT) in v8 protocol. Scale bars: 100 μm. FIG. 12E shows immunofluorescence staining of graft tissue recovered 8 weeks after transplantation of (v4)SC-islet clusters.

FIGS. 13A-13D demonstrates purification of SC-beta cells with ITGA1/CD49a. FIG. 13A shows expression of ITGA1/CD49a in Stage 6 time-course data. FIG. 13B provides immunofluorescence for SC-beta (top) and endocrine (bottom) markers of native, unsorted re-aggregated and CD49a+ sorted re-aggregated clusters. Scale bars: 100 μm. FIG. 13C provides flow cytometry quantification of SC-beta cells (C-pep+/NKX6.1+) and SC-EC cells (SLC18A1+) fractions in three matched conditions for 5 biologically independent v8 differentiations. FIG. 13D provides stimulation index for the same differentiations. In (FIGS. 13C-13D), symbol shows mean and error bars (where shown) correspond to standard errors across 3 independently-reaggregated biological replicates. P-values are from (two-sided) dependent t-test.

FIGS. 14A-14I provide a high-resolution map of in vitro endocrine induction. FIGS. 14A-14C shows tSNE projection of 51,274 cells, shaded according to (FIG. 14A) sampling time within Stage 5, (FIG. 14B) NEUROG3 expression and (FIG. 14C) assigned cell types. Arrows on FIG. 5C indicate key lineage bifurcations. FIG. 14D shows fraction of cells from each cluster in FIG. 14C for each day of both independent differentiations. FIG. 14E show tSNE shading of branch assignment and pseudotime value of each cell on the path from NKX6.1+ progenitors to SC-beta and SC-EC cells. FIG. 14F provides expression of selected marker genes along pseudotime ordering from FIG. 14E. Dots show expression in single cells, sorted and shaded according to pseudotime order. Lines show regression on pseudotime for each branch (blue: SC-EC, purple: SC-beta). FIG. 14G shows genes with significant branch-specific expression pattern. q-values are FDR adjusted (alpha=0.001) p-values from likelihood ratio test comparing branched and non-branched models (see methods). FIG. 14H provides mean expression values of transcription factors for clusters presented in FIGS. 14C-14D. Shading displays mean expression (z-normalized tpm) and diameter denotes fractional expression. FIG. 14I provides proposed developmental model for the key cell types produced by SC-beta protocol.

FIGS. 15A-15M provide comparison of two SC-beta protocol variants and resulting cell types. FIGS. 15A-15C provide immunofluorescence imaging of differentiated (v8, Stage 6, day 13)SC-islets showing staining of relevant markers. FIG. 15A shows SC-beta cells, typically positioned in the periphery, are positive for both NKX6.1 and C-peptide (fragment of proinsulin). FIG. 15B shows SC-EC cells are positive for SLC18A1, an enterochromaffin cell marker. These cells are also present in the periphery. FIG. 15C shows non-endocrine cells, marked by SOX9, are most commonly found near the center of SC-islets. Scale bars: 100 μm. FIGS. 15D-15E show summary of changes in Stages 3 and 4 in protocols x1 (FIG. 15D) and x2 (FIG. 15E) and representative flow cytometry results at the end of Stages 4 and 6. FIGS. 15F-15I provides tSNE projection of cells sampled from the ends of Stages 3-6 of protocol x2. Cells in FIGS. 15F-15I are colored according to their assigned cluster. Horizontal bars indicate cell type proportions. (Related to FIGS. 10D-10G). FIG. 15J provides a comparison of cell populations from protocols x1 and x2. Correlation is computed using the z-scores of mean tpm values (for each cluster) of 2000 high-variance genes. Rows and columns are ordered using hierarchical clustering. Cells are labeled as in FIGS. 15F-15I and FIGS. 10D-10G. FIGS. 15K-15L provide tSNE projections of Stage 6 from three differentiations, colored by cell type (FIG. 15K) and by differentiation (FIG. 15L). FIG. 15M provides correlation of cell populations derived from HUES8 (ES cells, v4 and x3) and iPS1016/31 (iPS cells, v4). Same colors as in FIG. 15K. Correlation is computed as in FIG. 15J.

FIGS. 16A-16C provide a functional assay of glucose stimulated insulin secretion (GSIS) during Stage 6 time course. FIG. 16A provides a design for a sequential GSIS assay. FIG. 16B provides the complete data for 3 independent flasks, assayed across several weeks. Circles are individual technical triplicates and bars show mean of those triplicates. FIG. 16C provides the complete data for cadaveric human islets 7 donors, run alongside samples from FIG. 16B.

FIGS. 17A-17F demonstrate Stage 6 SC-beta cells express characteristic beta cell markers. FIGS. 17A-17B provide tSNE projections of Stage 6 time course data shaded by sampling time (FIG. 17A) and by representative marker genes (FIG. 17B). Expression is normalized relative to maximum value and smoothed over neighboring cells. FIG. 17C provides expression profiles for key genes necessary for beta-cell function. Shading displays mean expression (tpm, log-scaled) and diameter denotes fractional expression. FIGS. 17D-17E provide comparisons of global expression between human cadaveric islet-derived beta cells and in vitro progenitors (FIG. 17D) and SC-beta cells (FIG. 17E). Note the shift in gene expression from progenitors to SC-beta cells. All genes shown in all panels from FIG. 17C are circled in red. FIG. 17F provide results from Gene Set Enrichment Analysis (GSEA) showing that gene sets from FIG. 17C are significantly upregulated during differentiation. Value plotted is −log 10 of the GSEA-reported FDR q-value (capped at 10), with sign showing direction of effect (i.e, purple positive values are up-regulated in SC-beta cells compared to NKX6.1 progenitors).

FIGS. 18A-18D provide comparison of SC-beta and SC-alpha cells to each other and their islet counterparts. FIG. 18A shows insulin and glucagon expression in SC-beta (purple distributions) and SC-alpha cells (red distributions) during several weeks of Stage 6, shown as violin plots of SC-beta or SC-alpha cells from that particular time point. Connected line connects medians of each population at each time point. FIG. 18B shows identification of genes enriched in cadaveric islet alpha cells and islet beta cells from data in Baron et al. 2016. FIG. 18C provides a heatmap of expression level of genes from FIG. 18B, shown for islet alpha, SC-alpha, SC-beta and islet beta cells. FIG. 18D shows genes enriched in islet beta cells are up-regulated in SC-beta cells, and genes enriched in alpha cells are up-regulated in SC-alpha cells. The displayed p-value is computed using a (two-sided) Wilcoxon rank-sum test. In boxplot: boxes extend from first to third quartiles, whiskers extend from 5^{th} to 95^{th} percentiles, central line indicates median and box notching indicates 95^{th} percentile confidence interval for median.

FIGS. 19A-19F demonstrate SC-EC cells secrete serotonin and exist in other protocols. FIG. 19A provides a schematic of serotonin synthesis from tryptophan. Enterochromaffin cells use TPH1, whereas serotoninergic neurons use TPH2 for the first and rate limiting synthesis step. FIG. 19B shows serotonin release during sequential challenges of low and high glucose followed by KCl depolarization. Upper panel: clusters from three independent SC-beta differentiation. Lower panel: human cadaveric islets from two donors. Symbols show values of individual replicates for each sample (different clusters from the same sample are split and measured separately). p-values computed using (two-sided) Wilcoxon rank-sum test (n.s=non-significant with p>0.05). FIGS. 19C-19D show expression of EC marker genes (shown in blue) is detectable in bulk RNA-sequencing (from Gupta et al.), and enriched via sorting of NKX6.1(GFP)+ cells, shown as fold-change, mean expression and differential expression q-values. Positive fold-change indicates higher expression in NKX6.1(GFP)+ cells. Enrichment of SC-EC markers is comparable to beta cell markers (shown in purple) and opposite of alpha cell markers (shown in red). All values shown are directly reproduced from results computed and deposited by Gupta et al. 2018. FIG. 19E provides flow cytometry showing that SLC18A1 is co-expressed with NKX6.1+ in SC-EC cells of v8 SC-beta protocol differentiations. This example is representative across more than one hundred independent differentiations. FIG. 19F provides a comparison of gene expression between WT mouse islets and mouse islets 25 weeks after beta-cell specific PRC2 ablation via EED knockout. Purple genes are example down-regulated beta cell identity genes, blue genes represent serotonin/EC signature. q-values are FDR-corrected (alpha=0.05) p-values from Limma differential expression analysis.

FIGS. 20A-20D provide characterization of non-endocrine cells from Stage 6 time course. FIGS. 20A-20B provide tSNE projections of non-endocrine cells from Stage 6 time course, shaded by collection day (FIG. 20A) or by genes relevant to cell identity (FIG. 20B). Expression is normalized relative to maximum value, and smoothed over neighboring cells. FIG. 20C provides a tSNE projection shaded by assigned cluster and bar charts of cellular fraction in each cluster by week of differentiation. FIG. 20D shows gene expression of population specific markers for each subpopulation of non-endocrine cells. Shading displays mean expression (z-normalized tpm) and diameter denotes fractional expression.

FIGS. 21A-21K demonstrate re-aggregation is a scalable, function-preserving method to enrich for endocrine cells. FIG. 21A provides a schematic drawing of a re-aggregation procedure to remove non-endocrine cells. Cells are enzymatically dissociated and re-aggregated during continued suspension culture. Non-endocrine cells fail to adhere and are removed by filtration. FIG. 21B provides a schematic of a CD49a enrichment procedure to produce SC-beta enriched clusters. Dissociated cells are stained with anti-CD49a PE-conjugated antibody, incubated with anti-PE magnetic microbeads and magnetically separated. The enriched cells are re-aggregated in 6 well plates on a rocker. FIG. 21C provides a tSNE projection of cells sequenced from native and re-aggregated clusters from a single differentiation showing strong depletion of the non-endocrine population. Cells in both panels were differentiated with protocol v8. FIG. 21D shows immunofluorescence staining for C-peptide, GCG and SLC18A1 showing distinct neighborhoods in re-aggregated clusters (protocol v8). Images shown are maximum intensity projections from z-stacks. Each panel shows separate, representative clusters stained for all markers. Scale bars: 100 μm. FIGS. 21E-21F show representative flow cytometry analysis of endocrine cell abundance (from protocol v8), before and after re-aggregation. Endocrine cells express CHGA. FIG. 21G shows a summary of population composition (as assayed by flow cytometry) in 60 re-aggregated (RA) and 41 native independent differentiations, carried out with protocol v8. Re-aggregations were carried out in spinner flasks. p-value computed using (two-sided) Wilcoxon rank-sum test. In FIG. 21G and FIG. 21H boxplots: boxes extend from first to third quartiles, whiskers extend from 5^{th} to 95^{th} percentiles, central line indicates median and box notching indicates 95^{th} percentile confidence interval for median. FIG. 21H provides a stimulation index (insulin released at 20 mM glucose/insulin released at 2 mM) of 52 independent protocol v8 differentiations, with paired native vs. re-aggregated comparisons. p-value computed using (two-sided) Wilcoxon signed-rank test. FIG. 21I provides complete data for static glucose stimulated insulin secretion assays, performed as in FIG. 16, corresponding to stimulation indices shown in FIG. 13D. Circles are individual technical triplicates and bars show mean of those triplicates. FIG. 21J shows dynamic perifusion assay of glucose responsive insulin secretion of human islets, native SC-beta clusters (Stage 6, day 22, v8) and matched CD49a magnetically sorted enriched SC-beta islets. Each point is the mean of 3 technical replicates, with the vertical bar indicating standard error across those triplicates. FIG. 21K shows area under the curve comparing the first low-glucose stimulation and the high-glucose stimulation, normalized to equal effective time in each treatment.

FIGS. 22A-22F demonstrate Stage 5 time course markers and progenitor population heterogeneity. FIGS. 22A-22B provide tSNE projections of Stage 5 time course data shaded by collection day (FIG. 22A) and by population marker genes (FIG. 22B). Expression is normalized relative to maximum value, and smoothed over neighboring cells. FIG. 22C shows a pseudotime analysis of day 0 (top) and day 1 (bottom) progenitor cells. Shading on each tSNE shows assigned pseudotime value of each cell. FIG. 22D shows pseudotime ordering of progenitor cells from Stage 5 day 0 (top row) and day 1 (bottom row) showing population heterogeneity among early progenitors. Individual cells are shown as dots, shaded as in (FIG. 22C). Gene expression predicted from pseudotime regression shown as overlaid line. FIG. 22E provides a summary of Stage 5 day 0 heterogeneity captured by pseudotime analysis. Fold-change between start and end of pseudotime ordering. q-value from likelihood ratio test of model with and without pseudotime. FIG. 22F provides a heatmap of receptors, ligands and signaling effectors that are dynamically expressed across Stage 5 populations. Shading displays mean expression (z-normalized tpm) and diameter denotes fractional expression.

FIG. 23 provides expression of key marker genes across all populations from time course datasets and cadaveric islets. Column on the left indicates origin dataset. Shading displays mean expression (z-normalized tpm) and diameter denotes fractional expression.

FIG. 24 provides expression of intestinal enteroendocrine marker genes across all populations from time course datasets. Column on the left indicates dataset origin. Shading displays mean expression (z-normalized tpm) and diameter denotes fractional expression.

FIGS. 25A-25D demonstrate an example of flow cytometry gating strategy. FIGS. 25A-25C show Stage 6 time course differentiation 1 (internal ID: DA-089), at Stage 6, day 13 of v8 protocol. FIG. 25A: Secondaries-only control. FIG. 25B: SC-beta cell identification via staining for C-peptide and NKX6.1. FIG. 25C: Endocrine cell identification via staining for CHGA and NKX6.1. Results are representative across more than a hundred v8 differentiations, with typical SC-beta percentages being 25-45%. FIG. 25D provides an example CD49a+ magnetic purification. Left panel shows CD49a+ distribution prior to sorting, right panel shows distribution after one round of magnetic separation (see methods). Results are representative across more than 10 enrichment experiments.

FIG. 26 provides specification of differentiation protocols used in the study. Summary of the different versions of the SC-beta protocol used throughout this study.

FIG. 27 provides a summary of all cell populations identified in the study. For each population, key markers for their identification, which datasets they were identified in and, for rare populations, a description of their relation to other populations are listed.

FIG. 28 provides a summary of single-cell RNA sequencing datasets generated in the study. This table specifies protocols, cell lines, number of inDrops libraries, source of inDrops reagents and number of cells sequenced for each dataset in the study, as well as the corresponding figures.

FIG. 29 demonstrates the homozygous deletion of FBXL14 increases the percentage of SC-beta cells. The figure provides a comparison of HUES8 ESC monoclonal lines on their endocrine cell formation ability. Three genotypes are compared (WT: wild-type, FBXL14 KO: homozygous deletion of FBXL14, NEUROG3 KO: homozygous deletion of NEUROG3) and each point is an independent differentiation. FBXL14 leads to 50% increase in proportion of SC-beta cells, relative to WT. NEUROG3 KO is a control showing complete loss of endocrine cell formation.

FIG. 30 demonstrates that overexpression of screen hit transcription factors (TFs) changes endocrine cell ratios. ISL1 overexpression increases SC-beta cell and SC-alpha cell while reducing SC-EC cell formation. Lentiviruses for constitutive expression of GFP and specified TF were produced, and separately delivered to whole clusters. 1-5% of the outer layer of cells was transduced. At S6d1, SC-islets were dissociated, fixed and stained for flow cytometry. The GFP+ fraction was specifically compared between target genes and a neutral control (carrying LUC2) to compute log 2 fold-change.

### DETAILED DESCRIPTION OF THE INVENTION

Aspects of the disclosure relate to methods of directing the differentiation of cells with multiple potential differentiation outcomes toward or away from particular differentiation outcomes.

### Definitions

For convenience, certain terms employed herein, in the specification, examples and appended claims are collected here. Unless otherwise defined, all technical and scientific terms used herein have the same meaning as commonly understood by one of ordinary skill in the art to which this invention belongs.

The term “differentiated cell” is meant any primary cell that is not, in its native form, pluripotent as that term is defined herein. Stated another way, the term “differentiated cell” refers to a cell of a more specialized cell type derived from a cell of a less specialized cell type (e.g., a stem cell such as an induced pluripotent stem cell) in a cellular differentiation process. Without wishing to be limited to theory, a pluripotent stem cell in the course of normal ontogeny can differentiate first to an endoderm cell that is capable of forming pancreas cells and other endoderm cell types. Further differentiation of an endoderm cell leads to the pancreatic pathway, where ^{˜}98% of the cells become exocrine, ductular, or matrix cells, and ^{˜}2% become endocrine cells.

As used herein, the term “somatic cell” refers to any cells forming the body of an organism, as opposed to germline cells. In mammals, germline cells (also known as “gametes”) are the spermatozoa and ova which fuse during fertilization to produce a cell called a zygote, from which the entire mammalian embryo develops. Every other cell type in the mammalian body—apart from the sperm and ova, the cells from which they are made (gametocytes) and undifferentiated stem cells—is a somatic cell: internal organs, skin, bones, blood, and connective tissue are all made up of somatic cells. In some embodiments the somatic cell is a “non-embryonic somatic cell”, by which is meant a somatic cell that is not present in or obtained from an embryo and does not result from proliferation of such a cell in vitro. In some embodiments the somatic cell is an “adult somatic cell”, by which is meant a cell that is present in or obtained from an organism other than an embryo or a fetus or results from proliferation of such a cell in vitro. Unless otherwise indicated the methods described herein can be performed both in vivo and in vitro.

As used herein, the term “adult cell” refers to a cell found throughout the body after embryonic development.

The term “endoderm cell” as used herein refers to a cell which is from one of the three primary germ cell layers in the very early embryo (the other two germ cell layers are the mesoderm and ectoderm). The endoderm is the innermost of the three layers. An endoderm cell differentiates to give rise first to the embryonic gut and then to the linings of respiratory and digestive tracts (e.g. the intestine), the liver and the pancreas.

The term “a cell of endoderm origin” as used herein refers to any cell which has developed or differentiated from an endoderm cell. For example, a cell of endoderm origin includes cells of the liver, lung, pancreas, thymus, intestine, stomach and thyroid. Without wishing to be bound by theory, liver and pancreas progenitors (also referred to as pancreatic progenitors) develop from endoderm cells in the embryonic foregut. Shortly after their specification, liver and pancreas progenitors rapidly acquire markedly different cellular functions and regenerative capacities. These changes are elicited by inductive signals and genetic regulatory factors that are highly conserved among vertebrates.

The term “pancreatic progenitor” or “pancreatic precursor” are used interchangeably herein and refer to a stem cell which is capable of forming any of pancreatic endocrine cells, pancreatic exocrine cells, or pancreatic duct cells. The term “Pdx1-positive pancreatic progenitor” or “Pdx1+ pancreatic progenitor” as used herein refers to a cell which is a pancreatic endoderm (PE) cell. A Pdx1-positive pancreatic progenitor expresses the marker Pdx1. Other markers include, but are not limited to Cdcp1, or Ptf1a, or HNF6 or NRx2.2. The expression of Pdx1 may be assessed by any method known by the skilled person such as immunochemistry using an anti-Pdx1 antibody or quantitative RT-PCR. The term “Pdx1-positive, NKX6-1-positive pancreatic progenitor” or “Pdx1+, NKX6-1+ pancreatic progenitor” as used herein refers to a cell which is a pancreatic endoderm (PE) cell. A Pdx1-positive, NKX6-1-positive pancreatic progenitor expresses the markers Pdx1 and NKX6-1. Other markers include, but are not limited to Cdcp1, or Ptf1a, or HNF6 or NRx2.2. The expression of NKX6-1 may be assessed by any method known by the skilled person such as immunochemistry using an anti-NKX6-1 antibody or quantitative RT-PCR.

The terms “stem cell-derived β cell”, “SC-β cell”, and “mature SC-β cell” refer to cells (e.g., pancreatic β cells) that display at least one marker indicative of a pancreatic β cell, express insulin, and display a GSIS response characteristic of an endogenous mature β cell. In some embodiments, the “SC-β cell” comprises a mature pancreatic β cell. It is to be understood that the SC-β cells need not be derived (e.g., directly) from stem cells, as the methods of the disclosure are capable of deriving SC-β cells from any insulin-positive endocrine cell or precursor thereof using any cell as a starting point (e.g., one can use embryonic stem cells, induced-pluripotent stem cells, progenitor cells, partially reprogrammed somatic cells (e.g., a somatic cell which has been partially reprogrammed to an intermediate state between an induced pluripotent stem cell and the somatic cell from which it was derived), multipotent cells, totipotent cells, a transdifferentiated version of any of the foregoing cells, etc., as the invention is not intended to be limited in this manner). Moreover, it should be understood that an SC-β cell of the invention is a non-native, i.e., non-naturally occurring, non-endogenous, cell and has at least one characteristic that is different from a native/naturally-occurring/endogenous cell. Examples of SC-β cells, and methods of obtaining such SC-β cells, are described in WO 2015/002724 and WO 2014/201167, both of which are incorporated herein by reference in their entirety.

The terms “stem cell-derived a cell”, “SC-α cell”, and “mature SC-α cell” refer to cells (e.g., pancreatic a cells) that display at least one marker indicative of a pancreatic a cell, express and secrete glucagon, and display an ultrastructure similar to cadaveric alpha cells. In some embodiments, the “SC-α cell” comprises a mature pancreatic a cell. It is to be understood that the SC-α cells need not be derived (e.g., directly) from stem cells, as the methods of the disclosure are capable of deriving SC-a cells from any insulin-positive endocrine cell or precursor thereof using any cell as a starting point (e.g., one can use embryonic stem cells, induced-pluripotent stem cells, progenitor cells, partially reprogrammed somatic cells (e.g., a somatic cell which has been partially reprogrammed to an intermediate state between an induced pluripotent stem cell and the somatic cell from which it was derived), multipotent cells, totipotent cells, a transdifferentiated version of any of the foregoing cells, etc., as the invention is not intended to be limited in this manner). Moreover, it should be understood that an SC-α cell of the invention is a non-native, i.e., non-naturally occurring, non-endogenous, cell and has at least one characteristic that is different from a native/naturally-occurring/endogenous cell. Examples of SC-α cells, and methods of obtaining such SC-α cells, are described in WO 2019/217487, which is incorporated herein by reference in its entirety.

The terms “stem cell-derived enterochromaffin cell,” “SC-EC cell,” and “mature SC-EC cell” refer to cells (e.g., enterochromaffin cells) that display at least one marker indicative of an enterochromaffin cell, express SLC18A1, and is capable of producing and releasing serotonin (5-HT). In some embodiments, the “SC-EC cell” comprises a mature enterochromaffin cell. It is to be understood that the SC-EC cells need not be derived (e.g., directly) from stem cells, as the methods of the disclosure are capable of deriving SC-EC cells from any progenitor cell using any cell as a starting point (e.g., one can use embryonic stem cells, induced-pluripotent stem cells, progenitor cells, partially reprogrammed somatic cells (e.g., a somatic cell which has been partially reprogrammed to an intermediate state between an induced pluripotent stem cell and the somatic cell from which it was derived), multipotent cells, totipotent cells, a transdifferentiated version of any of the foregoing cells, etc, as the invention is not intended to be limited in this manner). Moreover, it should be understood that an SC-α cell of the invention is a non-native, i.e., non-naturally occurring, non-endogenous, cell and has at least one characteristic that is different from a native/naturally-occurring/endogenous cell. Examples of SC-EC cells, and methods of obtaining such SC-EC cells, are described in WO 2019/217493, which is incorporated herein by reference in its entirety.

The terms “triple negative cell” or “TN cell” refer to cells that may exist in a cluster of differentiated pancreatic cells, but do not express INS, GCG, and SLC18A1. TN cells may include progenitor cells or stem cell-derived cells that are not SC-β cells, SC-α cells, or SC-EC cells.

The term “exocrine cell” as used herein refers to a cell of an exocrine gland, i.e. a gland that discharges its secretion via a duct. In particular embodiments, an exocrine cell refers to a pancreatic exocrine cell, which is a pancreatic cell that produces enzymes that are secreted into the small intestine. These enzymes help digest food as it passes through the gastrointestinal tract. Pancreatic exocrine cells are also known as islets of Langerhans, that secrete two hormones, insulin and glucagon.

The term “phenotype” refers to one or a number of total biological characteristics that define the cell or organism under a particular set of environmental conditions and factors, regardless of the actual genotype.

The term “pluripotent” as used herein refers to a cell with the capacity, under different conditions, to differentiate to more than one differentiated cell type, and preferably to differentiate to cell types characteristic of all three germ cell layers.

Pluripotent cells are characterized primarily by their ability to differentiate to more than one cell type, preferably to all three germ layers, using, for example, a nude mouse teratoma formation assay. Pluripotency is also evidenced by the expression of embryonic stem (ES) cell markers, although the preferred test for pluripotency is the demonstration of the capacity to differentiate into cells of each of the three germ layers. It should be noted that simply culturing such cells does not, on its own, render them pluripotent. Reprogrammed pluripotent cells (e.g. iPS cells as that term is defined herein) also have the characteristic of the capacity of extended passaging without loss of growth potential, relative to primary cell parents, which generally have capacity for only a limited number of divisions in culture.

As used herein, the terms “iPS cell” and “induced pluripotent stem cell” are used interchangeably and refers to a pluripotent stem cell artificially derived (e.g., induced or by complete reversal) from a non-pluripotent cell, typically an adult somatic cell, for example, by inducing a forced expression of one or more genes.

The term “progenitor” or “precursor” cell are used interchangeably herein and refer to cells that have a cellular phenotype that is more primitive (i.e., is at an earlier step along a developmental pathway or progression than is a fully differentiated cell) relative to a cell which it can give rise to by differentiation. Often, progenitor cells also have significant or very high proliferative potential. Progenitor cells can give rise to multiple distinct differentiated cell types or to a single differentiated cell type, depending on the developmental pathway and on the environment in which the cells develop and differentiate.

The term “stem cell” as used herein, refers to an undifferentiated cell which is capable of proliferation and giving rise to more progenitor cells having the ability to generate a large number of mother cells that can in turn give rise to differentiated, or differentiable daughter cells. The daughter cells themselves can be induced to proliferate and produce progeny that subsequently differentiate into one or more mature cell types, while also retaining one or more cells with parental developmental potential. The term “stem cell” refers to a subset of progenitors that have the capacity or potential, under particular circumstances, to differentiate to a more specialized or differentiated phenotype, and which retains the capacity, under certain circumstances, to proliferate without substantially differentiating. In one embodiment, the term stem cell refers generally to a naturally occurring mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell may derive from a multipotent cell which itself is derived from a multipotent cell, and so on. While each of these multipotent cells may be considered stem cells, the range of cell types each can give rise to may vary considerably. Some differentiated cells also have the capacity to give rise to cells of greater developmental potential. Such capacity may be natural or may be induced artificially upon treatment with various factors. In many biological instances, stem cells are also “multipotent” because they can produce progeny of more than one distinct cell type, but this is not required for “stem-ness.” Self-renewal is the other classical part of the stem cell definition, and it is essential as used in this document. In theory, self-renewal can occur by either of two major mechanisms. Stem cells may divide asymmetrically, with one daughter retaining the stem state and the other daughter expressing some distinct other specific function and phenotype. Alternatively, some of the stem cells in a population can divide symmetrically into two stems, thus maintaining some stem cells in the population as a whole, while other cells in the population give rise to differentiated progeny only. Formally, it is possible that cells that begin as stem cells might proceed toward a differentiated phenotype, but then “reverse” and re-express the stem cell phenotype, a term often referred to as “dedifferentiation” or “reprogramming” or “retrodifferentiation” by persons of ordinary skill in the art. As used herein, the term “pluripotent stem cell” includes embryonic stem cells, induced pluripotent stem cells, placental stem cells, etc.

The term “embryonic stem cell” is used to refer to the pluripotent stem cells of the inner cell mass of the embryonic blastocyst (see U.S. Pat. Nos. 5,843,780, 6,200,806). Such cells can similarly be obtained from the inner cell mass of blastocysts derived from somatic cell nuclear transfer (see, for example, U.S. Pat. Nos. 5,945,577, 5,994,619, 6,235,970). The distinguishing characteristics of an embryonic stem cell define an embryonic stem cell phenotype. Accordingly, a cell has the phenotype of an embryonic stem cell if it possesses one or more of the unique characteristics of an embryonic stem cell such that that cell can be distinguished from other cells. Exemplary distinguishing embryonic stem cell characteristics include, without limitation, gene expression profile, proliferative capacity, differentiation capacity, karyotype, responsiveness to particular culture conditions, and the like.

The term “adult stem cell” or “ASC” is used to refer to any multipotent stem cell derived from non-embryonic tissue, including fetal, juvenile, and adult tissue. Stem cells have been isolated from a wide variety of adult tissues including blood, bone marrow, brain, olfactory epithelium, skin, pancreas, skeletal muscle, and cardiac muscle. Each of these stem cells can be characterized based on gene expression, factor responsiveness, and morphology in culture. Exemplary adult stem cells include neural stem cells, neural crest stem cells, mesenchymal stem cells, hematopoietic stem cells, and pancreatic stem cells. As indicated above, stem cells have been found resident in virtually every tissue. Accordingly, the present invention appreciates that stem cell populations can be isolated from virtually any animal tissue.

The term “reprogramming” as used herein refers to the process that alters or reverses the differentiation state of a somatic cell. The cell can either be partially or terminally differentiated prior to the reprogramming. Reprogramming encompasses complete reversion of the differentiation state of a somatic cell to a pluripotent cell. Such complete reversal of differentiation produces an induced pluripotent (iPS) cell. Reprogramming as used herein also encompasses partial reversion of a cells differentiation state, for example to a multipotent state or to a somatic cell that is neither pluripotent or multipotent, but is a cell that has lost one or more specific characteristics of the differentiated cell from which it arises, e.g. direct reprogramming of a differentiated cell to a different somatic cell type. Reprogramming generally involves alteration, e.g., reversal, of at least some of the heritable patterns of nucleic acid modification (e.g., methylation), chromatin condensation, epigenetic changes, genomic imprinting, etc., that occur during cellular differentiation as a zygote develops into an adult.

The term “agent” as used herein means any compound or substance such as, but not limited to, a small molecule, nucleic acid, polypeptide, peptide, drug, ion, etc. An “agent” can be any chemical, entity or moiety, including without limitation synthetic and naturally-occurring proteinaceous and non-proteinaceous entities. In some embodiments, an agent is nucleic acid, nucleic acid analogues, proteins, antibodies, peptides, aptamers, oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof etc. In certain embodiments, agents are small molecule having a chemical moiety. For example, chemical moieties included unsubstituted or substituted alkyl, aromatic, or heterocyclyl moieties including macrolides, leptomycins and related natural products or analogues thereof. Compounds can be known to have a desired activity and/or property, or can be selected from a library of diverse compounds.

As used herein, the term “contacting” (i.e., contacting at least one endocrine cell or a precursor thereof with a maturation factor, or combination of maturation factors) is intended to include incubating the maturation factor and the cell together in vitro (e.g., adding the maturation factors to cells in culture). In some embodiments, the term “contacting” is not intended to include the in vivo exposure of cells to the compounds as disclosed herein that may occur naturally in a subject (i.e., exposure that may occur as a result of a natural physiological process). The step of contacting at least one endocrine cell or a precursor thereof with a maturation factor as in the embodiments described herein can be conducted in any suitable manner. For example, the cells may be treated in adherent culture, or in suspension culture. In some embodiments, the cells are treated in conditions that promote cell clustering. The disclosure contemplates any conditions which promote cell clustering. Examples of conditions that promote cell clustering include, without limitation, suspension culture in low attachment tissue culture plates, spinner flasks, or aggrewell plates. In some embodiments, the inventors have observed that clusters have remained stable in media containing 10% serum. In some embodiments, the conditions that promote clustering include a low serum medium.

It is understood that the cells contacted with a maturation factor can also be simultaneously or subsequently contacted with another agent, such as a growth factor or other differentiation agent or environments to stabilize the cells, or to differentiate the cells further.

The term “cell culture medium” (also referred to herein as a “culture medium” or “medium”) as referred to herein is a medium for culturing cells containing nutrients that maintain cell viability and support proliferation. The cell culture medium may contain any of the following in an appropriate combination: salt(s), buffer(s), amino acids, glucose or other sugar(s), antibiotics, serum or serum replacement, and other components such as peptide growth factors, etc. Cell culture media ordinarily used for particular cell types are known to those skilled in the art.

The term “cell line” refers to a population of largely or substantially identical cells that has typically been derived from a single ancestor cell or from a defined and/or substantially identical population of ancestor cells. The cell line may have been or may be capable of being maintained in culture for an extended period (e.g., months, years, for an unlimited period of time). It may have undergone a spontaneous or induced process of transformation conferring an unlimited culture lifespan on the cells. Cell lines include all those cell lines recognized in the art as such. It will be appreciated that cells acquire mutations and possibly epigenetic changes over time such that at least some properties of individual cells of a cell line may differ with respect to each other. In some embodiments, a cell line comprises a stem cell derived cell described herein.

The term “exogenous” refers to a substance present in a cell or organism other than its native source. For example, the terms “exogenous nucleic acid” or “exogenous protein” refer to a nucleic acid or protein that has been introduced by a process involving the hand of man into a biological system such as a cell or organism in which it is not normally found or in which it is found in lower amounts. A substance will be considered exogenous if it is introduced into a cell or an ancestor of the cell that inherits the substance. In contrast, the term “endogenous” refers to a substance that is native to the biological system.

The term “expression” refers to the cellular processes involved in producing RNA and proteins and as appropriate, secreting proteins, including where applicable, but not limited to, for example, transcription, translation, folding, modification and processing. “Expression products” include RNA transcribed from a gene and polypeptides obtained by translation of mRNA transcribed from a gene.

The terms “genetically modified” or “engineered” cell as used herein refers to a cell into which an exogenous nucleic acid has been introduced by a process involving the hand of man (or a descendant of such a cell that has inherited at least a portion of the nucleic acid). The nucleic acid may for example contain a sequence that is exogenous to the cell, it may contain native sequences (i.e., sequences naturally found in the cells) but in a non-naturally occurring arrangement (e.g., a coding region linked to a promoter from a different gene), or altered versions of native sequences, etc. The process of transferring the nucleic acid into the cell can be achieved by any suitable technique. Suitable techniques include calcium phosphate or lipid-mediated transfection, electroporation, and transduction or infection using a viral vector. In some embodiments the polynucleotide or a portion thereof is integrated into the genome of the cell. The nucleic acid may have subsequently been removed or excised from the genome, provided that such removal or excision results in a detectable alteration in the cell relative to an unmodified but otherwise equivalent cell. It should be appreciated that the term genetically modified is intended to include the introduction of a modified RNA directly into a cell (e.g., a synthetic, modified RNA). Such synthetic modified RNAs include modifications to prevent rapid degradation by endo- and exo-nucleases and to avoid or reduce the cell's innate immune or interferon response to the RNA. Modifications include, but are not limited to, for example, (a) end modifications, e.g., 5′ end modifications (phosphorylation dephosphorylation, conjugation, inverted linkages, etc.), 3′ end modifications (conjugation, DNA nucleotides, inverted linkages, etc.), (b) base modifications, e.g., replacement with modified bases, stabilizing bases, destabilizing bases, or bases that base pair with an expanded repertoire of partners, or conjugated bases, (c) sugar modifications (e.g., at the 2′ position or 4′ position) or replacement of the sugar, as well as (d) internucleoside linkage modifications, including modification or replacement of the phosphodiester linkages. To the extent that such modifications interfere with translation (i.e., results in a reduction of 50% or more in translation relative to the lack of the modification—e.g., in a rabbit reticulocyte in vitro translation assay), the modification is not suitable for the methods and compositions described herein.

The term “identity” as used herein refers to the extent to which the sequence of two or more nucleic acids or polypeptides is the same. The percent identity between a sequence of interest and a second sequence over a window of evaluation, e.g., over the length of the sequence of interest, may be computed by aligning the sequences, determining the number of residues (nucleotides or amino acids) within the window of evaluation that are opposite an identical residue allowing the introduction of gaps to maximize identity, dividing by the total number of residues of the sequence of interest or the second sequence (whichever is greater) that fall within the window, and multiplying by 100. When computing the number of identical residues needed to achieve a particular percent identity, fractions are to be rounded to the nearest whole number. Percent identity can be calculated with the use of a variety of computer programs known in the art. For example, computer programs such as BLAST2, BLASTN, BLASTP, Gapped BLAST, etc., generate alignments and provide percent identity between sequences of interest. The algorithm of Karlin and Altschul (Karlin and Altschul, Proc. Natl. Acad. Sci. USA 87:22264-2268, 1990) modified as in Karlin and Altschul, Proc. Natl. Acad. ScL USA 90:5873-5877, 1993 is incorporated into the NBLAST and XBLAST programs of Altschul et al. (Altschul, et al., J. MoI. Biol. 215:403-410, 1990). To obtain gapped alignments for comparison purposes, Gapped BLAST is utilized as described in Altschul et al. (Altschul, et al. Nucleic Acids Res. 25: 3389-3402, 1997). When utilizing BLAST and Gapped BLAST programs, the default parameters of the respective programs may be used. A PAM250 or BLOSUM62 matrix may be used. Software for performing BLAST analyses is publicly available through the National Center for Biotechnology Information (NCBI). See the Web site having URL world-wide web address of: “ncbi.nlm nih.gov” for these programs. In a specific embodiment, percent identity is calculated using BLAST2 with default parameters as provided by the NCBI.

The term “isolated” or “partially purified” as used herein refers, in the case of a nucleic acid or polypeptide, to a nucleic acid or polypeptide separated from at least one other component (e.g., nucleic acid or polypeptide) that is present with the nucleic acid or polypeptide as found in its natural source and/or that would be present with the nucleic acid or polypeptide when expressed by a cell, or secreted in the case of secreted polypeptides. A chemically synthesized nucleic acid or polypeptide or one synthesized using in vitro transcription/translation is considered “isolated”.

The term “isolated cell” as used herein refers to a cell that has been removed from an organism in which it was originally found or a descendant of such a cell. Optionally the cell has been cultured in vitro, e.g., in the presence of other cells. Optionally the cell is later introduced into a second organism or re-introduced into the organism from which it (or the cell from which it is descended) was isolated.

The term “isolated population” with respect to an isolated population of cells as used herein refers to a population of cells that has been removed and separated from a mixed or heterogeneous population of cells. In some embodiments, an isolated population is a substantially pure population of cells as compared to the heterogeneous population from which the cells were isolated or enriched from.

The term “substantially pure”, with respect to a particular cell population, refers to a population of cells that is at least about 75%, preferably at least about 85%, more preferably at least about 90%, and most preferably at least about 95% pure, with respect to the cells making up a total cell population.

The terms “enriching” or “enriched” are used interchangeably herein and mean that the yield (fraction) of cells of one type is increased by at least 10% over the fraction of cells of that type in the starting culture or preparation.

The terms “renewal” or “self-renewal” or “proliferation” are used interchangeably herein, and are used to refer to the ability of stem cells to renew themselves by dividing into the same non-specialized cell type over long periods, and/or many months to years. In some instances, proliferation refers to the expansion of cells by the repeated division of single cells into two identical daughter cells.

The term “lineages” as used herein describes a cell with a common ancestry or cells with a common developmental fate. For example, in the context of a cell that is of endoderm origin or is “endodermal linage” this means the cell was derived from an endoderm cell and can differentiate along the endoderm lineage restricted pathways, such as one or more developmental lineage pathways which give rise to definitive endoderm cells, which in turn can differentiate into liver cells, thymus, pancreas, lung and intestine.

A “marker” as used herein is used to describe the characteristics and/or phenotype of a cell. Markers can be used for selection of cells comprising characteristics of interests. Markers will vary with specific cells. Markers are characteristics, whether morphological, functional or biochemical (enzymatic) characteristics of the cell of a particular cell type, or molecules expressed by the cell type. Preferably, such markers are proteins, and more preferably, possess an epitope for antibodies or other binding molecules available in the art. However, a marker may consist of any molecule found in a cell including, but not limited to, proteins (peptides and polypeptides), lipids, polysaccharides, nucleic acids and steroids. Examples of morphological characteristics or traits include, but are not limited to, shape, size, and nuclear to cytoplasmic ratio. Examples of functional characteristics or traits include, but are not limited to, the ability to adhere to particular substrates, ability to incorporate or exclude particular dyes, ability to migrate under particular conditions, and the ability to differentiate along particular lineages. Markers may be detected by any method available to one of skill in the art. Markers can also be the absence of a morphological characteristic or absence of proteins, lipids etc. Markers can be a combination of a panel of unique characteristics of the presence and absence of polypeptides and other morphological characteristics.

The term “modulate” is used consistently with its use in the art, i.e., meaning to cause or facilitate a qualitative or quantitative change, alteration, or modification in a process, pathway, or phenomenon of interest. Without limitation, such change may be an increase, decrease, or change in relative strength or activity of different components or branches of the process, pathway, or phenomenon. A “modulator” is an agent that causes or facilitates a qualitative or quantitative change, alteration, or modification in a process, pathway, or phenomenon of interest.

As used herein, the term “DNA” is defined as deoxyribonucleic acid.

The term “polynucleotide” is used herein interchangeably with “nucleic acid” to indicate a polymer of nucleosides. Typically a polynucleotide of this invention is composed of nucleosides that are naturally found in DNA or RNA (e.g., adenosine, thymidine, guanosine, cytidine, uridine, deoxyadenosine, deoxythymidine, deoxyguanosine, and deoxycytidine) joined by phosphodiester bonds. However the term encompasses molecules comprising nucleosides or nucleoside analogs containing chemically or biologically modified bases, modified backbones, etc., whether or not found in naturally occurring nucleic acids, and such molecules may be preferred for certain applications. Where this application refers to a polynucleotide it is understood that both DNA, RNA, and in each case both single- and double-stranded forms (and complements of each single-stranded molecule) are provided. “Polynucleotide sequence” as used herein can refer to the polynucleotide material itself and/or to the sequence information (i.e. the succession of letters used as abbreviations for bases) that biochemically characterizes a specific nucleic acid. A polynucleotide sequence presented herein is presented in a 5′ to 3′ direction unless otherwise indicated.

The terms “polypeptide” as used herein refers to a polymer of amino acids. The terms “protein” and “polypeptide” are used interchangeably herein. A peptide is a relatively short polypeptide, typically between about 2 and 60 amino acids in length. Polypeptides used herein typically contain amino acids such as the 20 L-amino acids that are most commonly found in proteins. However, other amino acids and/or amino acid analogs known in the art can be used. One or more of the amino acids in a polypeptide may be modified, for example, by the addition of a chemical entity such as a carbohydrate group, a phosphate group, a fatty acid group, a linker for conjugation, functionalization, etc. A polypeptide that has a non-polypeptide moiety covalently or non-covalently associated therewith is still considered a “polypeptide”. Exemplary modifications include glycosylation and palmitoylation. Polypeptides may be purified from natural sources, produced using recombinant DNA technology, synthesized through chemical means such as conventional solid phase peptide synthesis, etc. The term “polypeptide sequence” or “amino acid sequence” as used herein can refer to the polypeptide material itself and/or to the sequence information (i.e., the succession of letters or three letter codes used as abbreviations for amino acid names) that biochemically characterizes a polypeptide. A polypeptide sequence presented herein is presented in an N-terminal to C-terminal direction unless otherwise indicated.

The term a “variant” in referring to a polypeptide could be, e.g., a polypeptide at least 80%, 85%, 90%, 95%, 98%, or 99% identical to full length polypeptide. The variant could be a fragment of full length polypeptide. The variant could be a naturally occurring splice variant. The variant could be a polypeptide at least 80%, 85%, 90%, 95%, 98%, or 99% identical to a fragment of the polypeptide, wherein the fragment is at least 50%, 60%, 70%, 80%, 85%, 90%, 95%, 98%, or 99% as long as the full length wild type polypeptide or a domain thereof having an activity of interest. In some embodiments the domain is at least 100, 200, 300, or 400 amino acids in length, beginning at any amino acid position in the sequence and extending toward the C-terminus. Variations known in the art to eliminate or substantially reduce the activity of the protein are preferably avoided. In some embodiments, the variant lacks an N- and/or C-terminal portion of the full length polypeptide, e.g., up to 10, 20, or 50 amino acids from either terminus is lacking. In some embodiments the polypeptide has the sequence of a mature (full length) polypeptide, by which is meant a polypeptide that has had one or more portions such as a signal peptide removed during normal intracellular proteolytic processing (e.g., during co-translational or post-translational processing). In some embodiments wherein the protein is produced other than by purifying it from cells that naturally express it, the protein is a chimeric polypeptide, by which is meant that it contains portions from two or more different species. In some embodiments wherein a protein is produced other than by purifying it from cells that naturally express it, the protein is a derivative, by which is meant that the protein comprises additional sequences not related to the protein so long as those sequences do not substantially reduce the biological activity of the protein.

The term “functional fragments” as used herein is a polypeptide having an amino acid sequence which is smaller in size than, but substantially homologous to the polypeptide it is a fragment of, and where the functional fragment polypeptide sequence is about at least 50%, or 60% or 70% or 80% or 90% or 100% or greater than 100%, for example 1.5-fold, 2-fold, 3-fold, 4-fold or greater than 4-fold effective biological action as the polypeptide from which it is a fragment of. Functional fragment polypeptides may have additional functions that can include decreased antigenicity, increased DNA binding (as in transcription factors), or altered RNA binding (as in regulating RNA stability or degradation).

The term “vector” refers to a carrier DNA molecule into which a DNA sequence can be inserted for introduction into a host cell. Preferred vectors are those capable of autonomous replication and/or expression of nucleic acids to which they are linked. Vectors capable of directing the expression of genes to which they are operatively linked are referred to herein as “expression vectors”. Thus, an “expression vector” is a specialized vector that contains the necessary regulatory regions needed for expression of a gene of interest in a host cell. In some embodiments the gene of interest is operably linked to another sequence in the vector. Vectors can be viral vectors or non-viral vectors. Should viral vectors be used, it is preferred the viral vectors are replication defective, which can be achieved for example by removing all viral nucleic acids that encode for replication. A replication defective viral vector will still retain its infective properties and enters the cells in a similar manner as a replicating adenoviral vector, however once admitted to the cell a replication defective viral vector does not reproduce or multiply. Vectors also encompass liposomes and nanoparticles and other means to deliver DNA molecule to a cell.

The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of coding sequences and transcription control elements (e.g. promoters, enhancers, and termination elements) in an expression vector. The term “operatively linked” includes having an appropriate start signal (e.g., ATG) in front of the polynucleotide sequence to be expressed, and maintaining the correct reading frame to permit expression of the polynucleotide sequence under the control of the expression control sequence, and production of the desired polypeptide encoded by the polynucleotide sequence.

The term “viral vectors” refers to the use of viruses, or virus-associated vectors as carriers of a nucleic acid construct into a cell. Constructs may be integrated and packaged into non-replicating, defective viral genomes like Adenovirus, Adeno-associated virus (AAV), or Herpes simplex virus (HSV) or others, including retroviral and lentiviral vectors, for infection or transduction into cells. The vector may or may not be incorporated into the cell's genome. The constructs may include viral sequences for transfection, if desired. Alternatively, the construct may be incorporated into vectors capable of episomal replication, e.g EPV and EBV vectors.

The terms “regulatory sequence” and “promoter” are used interchangeably herein, and refer to nucleic acid sequences, such as initiation signals, enhancers, and promoters, which induce or control transcription of protein coding sequences with which they are operatively linked. In some examples, transcription of a recombinant gene is under the control of a promoter sequence (or other transcriptional regulatory sequence) which controls the expression of the recombinant gene in a cell-type in which expression is intended. It will also be understood that the recombinant gene can be under the control of transcriptional regulatory sequences which are the same or which are different from those sequences which control transcription of the naturally-occurring form of a protein. In some instances, the promoter sequence is recognized by the synthetic machinery of the cell, or introduced synthetic machinery, required for initiating transcription of a specific gene.

As used herein, the term “transcription factor” refers to a protein that binds to specific parts of DNA using DNA binding domains and is part of the system that controls the transfer (or transcription) of genetic information from DNA to RNA. As used herein, “proliferating” and “proliferation” refer to an increase in the number of cells in a population (growth) by means of cell division. Cell proliferation is generally understood to result from the coordinated activation of multiple signal transduction pathways in response to the environment, including growth factors and other mitogens. Cell proliferation may also be promoted by release from the actions of intra- or extracellular signals and mechanisms that block or negatively affect cell proliferation.

The term “selectable marker” refers to a gene, RNA, or protein that when expressed, confers upon cells a selectable phenotype, such as resistance to a cytotoxic or cytostatic agent (e.g., antibiotic resistance), nutritional prototrophy, or expression of a particular protein that can be used as a basis to distinguish cells that express the protein from cells that do not. Proteins whose expression can be readily detected such as a fluorescent or luminescent protein or an enzyme that acts on a substrate to produce a colored, fluorescent, or luminescent substance (“detectable markers”) constitute a subset of selectable markers. The presence of a selectable marker linked to expression control elements native to a gene that is normally expressed selectively or exclusively in pluripotent cells makes it possible to identify and select somatic cells that have been reprogrammed to a pluripotent state. A variety of selectable marker genes can be used, such as neomycin resistance gene (neo), puromycin resistance gene (puro), guanine phosphoribosyl transferase (gpt), dihydrofolate reductase (DHFR), adenosine deaminase (ada), puromycin-N-acetyltransferase (PAC), hygromycin resistance gene (hyg), multidrug resistance gene (mdr), thymidine kinase (TK), hypoxanthine-guanine phosphoribosyltransferase (HPRT), and hisD gene. Detectable markers include green fluorescent protein (GFP) blue, sapphire, yellow, red, orange, and cyan fluorescent proteins and variants of any of these. Luminescent proteins such as luciferase (e.g., firefly or Renilla luciferase) are also of use. As will be evident to one of skill in the art, the term “selectable marker” as used herein can refer to a gene or to an expression product of the gene, e.g., an encoded protein.

In some embodiments the selectable marker confers a proliferation and/or survival advantage on cells that express it relative to cells that do not express it or that express it at significantly lower levels. Such proliferation and/or survival advantage typically occurs when the cells are maintained under certain conditions, i.e., “selective conditions.” To ensure an effective selection, a population of cells can be maintained under conditions and for a sufficient period of time such that cells that do not express the marker do not proliferate and/or do not survive and are eliminated from the population or their number is reduced to only a very small fraction of the population. The process of selecting cells that express a marker that confers a proliferation and/or survival advantage by maintaining a population of cells under selective conditions so as to largely or completely eliminate cells that do not express the marker is referred to herein as “positive selection”, and the marker is said to be “useful for positive selection”. Negative selection and markers useful for negative selection are also of interest in certain of the methods described herein. Expression of such markers confers a proliferation and/or survival disadvantage on cells that express the marker relative to cells that do not express the marker or express it at significantly lower levels (or, considered another way, cells that do not express the marker have a proliferation and/or survival advantage relative to cells that express the marker). Cells that express the marker can therefore be largely or completely eliminated from a population of cells when maintained in selective conditions for a sufficient period of time.

A “reporter gene” as used herein encompasses any gene that is genetically introduced into a cell that adds to the phenotype of the stem cell. Reporter genes as disclosed in this invention are intended to encompass fluorescent, luminescent, enzymatic and resistance genes, but also other genes which can easily be detected by persons of ordinary skill in the art. In some embodiments of the invention, reporter genes are used as markers for the identification of particular stem cells, cardiovascular stem cells and their differentiated progeny. A reporter gene is generally operatively linked to sequences that regulate its expression in a manner dependent upon one or more conditions which are monitored by measuring expression of the reporter gene.

In some cases, expression of the reporter gene may be determined in live cells. Where live cell reporter gene assays are used, reporter gene expression may be monitored at multiple time points, e.g., 2, 3, 4, 5, 6, 8, or 10 or more time points. In some cases, where a live cell reporter assay is used, reporter gene expression is monitored with a frequency of at least about 10 minutes to about 24 hours, e.g., 20 minutes, 1 hour, 2 hours, 3 hours, 4 hours, 5 hours, 6 hours, 7 hours, 8 hours, 9 hours, 10 hours, 12 hours, 18 hours, or another frequency from any integer between about 10 minutes to about 24 hours.

The terms “subject” and “individual” are used interchangeably herein, and refer to an animal, for example, a human from whom cells can be obtained and/or to whom treatment, including prophylactic treatment, with the cells as described herein, is provided. For treatment of those infections, conditions or disease states which are specific for a specific animal such as a human subject, the term subject refers to that specific animal. The “non-human animals” and “non-human mammals” as used interchangeably herein, includes mammals such as rats, mice, rabbits, sheep, cats, dogs, cows, pigs, and non-human primates. The term “subject” also encompasses any vertebrate including but not limited to mammals, reptiles, amphibians and fish. However, advantageously, the subject is a mammal such as a human, or other mammals such as a domesticated mammal, e.g. dog, cat, horse, and the like, or production mammal, e.g. cow, sheep, pig, and the like.

The terms “treat”, “treating”, “treatment”, etc., as applied to an isolated cell, include subjecting the cell to any kind of process or condition or performing any kind of manipulation or procedure on the cell. As applied to a subject, the terms refer to providing medical or surgical attention, care, or management to an individual. The individual is usually ill or injured, or at increased risk of becoming ill relative to an average member of the population and in need of such attention, care, or management.

As used herein, the term “treating” and “treatment” refers to administering to a subject an effective amount of a composition so that the subject as a reduction in at least one symptom of the disease or an improvement in the disease, for example, beneficial or desired clinical results. For purposes of this invention, beneficial or desired clinical results include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable. Treating can refer to prolonging survival as compared to expected survival if not receiving treatment. Thus, one of skill in the art realizes that a treatment may improve the disease condition, but may not be a complete cure for the disease. As used herein, the term “treatment” includes prophylaxis. Alternatively, treatment is “effective” if the progression of a disease is reduced or halted. “Treatment” can also mean prolonging survival as compared to expected survival if not receiving treatment.

As used herein, the terms “administering,” “introducing” and “transplanting” are used interchangeably in the context of the placement of cells of the invention into a subject, by a method or route which results in at least partial localization of the introduced cells at a desired site. The cells can be implanted directly to the pancreas or gastrointestinal tract, or alternatively be administered by any appropriate route which results in delivery to a desired location in the subject where at least a portion of the implanted cells or components of the cells remain viable. The period of viability of the cells after administration to a subject can be as short as a few hours, e.g. twenty-four hours, to a few days, to as long as several years. In some instances, the cells can also be administered subcutaneously, for example, in a capsule (e.g., microcapsule) to maintain the implanted cells at the implant location and avoid migration of the implanted cells.

The phrases “parenteral administration” and “administered parenterally” as used herein means modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, subcapsular, subarachnoid, intraspinal, intracerebro spinal, and intrasternal injection and infusion. The phrases “systemic administration,” “administered systemically”, “peripheral administration” and “administered peripherally” as used herein mean the administration of stem cell-derived cells and/or their progeny and/or compound and/or other material other than directly into the central nervous system, such that it enters the animal's system and, thus, is subject to metabolism and other like processes, for example, subcutaneous administration. The term “tissue” refers to a group or layer of specialized cells which together perform certain special functions. The term “tissue-specific” refers to a source of cells from a specific tissue.

The terms “decrease,” “reduced,” “reduction,” “decrease,” or “inhibit” are all used herein generally to mean a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The terms “increased,” “increase,” “enhance,” or “activate” are all used herein to generally mean an increase by a statically significant amount; for the avoidance of any doubt, the terms “increased,” “increase,” “enhance,” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “statistically significant” or “significantly” refers to statistical significance and generally means a two standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth.

### Stem Cells

Stem cells are cells that retain the ability to renew themselves through mitotic cell division and can differentiate into a diverse range of specialized cell types. The two broad types of mammalian stem cells are: embryonic stem (ES) cells that are found in blastocysts, and adult stem cells that are found in adult tissues. In a developing embryo, stem cells can differentiate into all of the specialized embryonic tissues. In adult organisms, stem cells and progenitor cells act as a repair system for the body, replenishing specialized cells, but also maintain the normal turnover of regenerative organs, such as blood, skin or intestinal tissues. Pluripotent stem cells can differentiate into cells derived from any of the three germ layers.

While certain embodiments are described below in reference to the use of stem cells, germ cells may be used in place of, or with, the stem cells to provide at least one differentiated cell, using similar protocols as the illustrative protocols described herein. Suitable germ cells can be prepared, for example, from primordial germ cells present in human fetal material taken about 8-11 weeks after the last menstrual period. Illustrative germ cell preparation methods are described, for example, in Shamblott et al., Proc. Natl. Acad. Sci. USA 95:13726, 1998 and U.S. Pat. No. 6,090,622.

ES cells, e.g., human embryonic stem cells (hESCs) or mouse embryonic stem cells (mESCs), with a virtually endless replication capacity and the potential to differentiate into most cell types, present, in principle, an unlimited starting material to generate the differentiated cells for clinical therapy (stemcells.nih.gov/info/scireport/2006report.htm, 2006).

hESC cells, are described, for example, by Cowan et al. (N Engl. J. Med. 350:1353, 2004) and Thomson et al. (Science 282:1145, 1998); embryonic stem cells from other primates, Rhesus stem cells (Thomson et al., Proc. Natl. Acad. Sci. USA 92:7844, 1995), marmoset stem cells (Thomson et al., Biol. Reprod. 55:254, 1996) and human embryonic germ (hEG) cells (Shamblott et al., Proc. Natl. Acad. Sci. USA 95:13726, 1998) may also be used in the methods disclosed herein. mESCs, are described, for example, by Tremml et al. (Curr Protoc Stem Cell Biol. Chapter 1:Unit 1C.4, 2008). The stem cells may be, for example, unipotent, totipotent, multipotent, or pluripotent. In some examples, any cells of primate origin that are capable of producing progeny that are derivatives of at least one germinal layer, or all three germinal layers, may be used in the methods disclosed herein.

In certain examples, ES cells may be isolated, for example, as described in Cowan et al. (N Engl. J. Med. 350:1353, 2004) and U.S. Pat. No. 5,843,780 and Thomson et al., Proc. Natl. Acad. Sci. USA 92:7844, 1995. For example, hESCs cells can be prepared from human blastocyst cells using the techniques described by Thomson et al. (U.S. Pat. No. 6,200,806; Science 282:1145, 1998; Curr. Top. Dev. Biol. 38:133 ff., 1998) and Reubinoff et al, Nature Biotech. 18:399, 2000. Equivalent cell types to hESCs include their pluripotent derivatives, such as primitive ectoderm-like (EPL) cells, as outlined, for example, in WO 01/51610 (Bresagen). hESCs can also be obtained from human pre-implantation embryos. Alternatively, in vitro fertilized (IVF) embryos can be used, or one-cell human embryos can be expanded to the blastocyst stage (Bongso et al., Hum Reprod 4: 706, 1989). Embryos are cultured to the blastocyst stage in G1.2 and G2.2 medium (Gardner et al., Fertil. Steril. 69:84, 1998). The zona pellucida is removed from developed blastocysts by brief exposure to pronase (Sigma). The inner cell masses can be isolated by immunosurgery, in which blastocysts are exposed to a 1:50 dilution of rabbit anti-human spleen cell antiserum for 30 min, then washed for 5 min three times in DMEM, and exposed to a 1:5 dilution of Guinea pig complement (Gibco) for 3 min (Solter et al., Proc. Natl. Acad. Sci. USA 72:5099, 1975). After two further washes in DMEM, lysed trophectoderm cells are removed from the intact inner cell mass (ICM) by gentle pipetting, and the ICM plated on mEF feeder layers. After 9 to 15 days, inner cell mass-derived outgrowths can be dissociated into clumps, either by exposure to calcium and magnesium-free phosphate-buffered saline (PBS) with 1 mM EDTA, by exposure to dispase or trypsin, or by mechanical dissociation with a micropipette; and then replated on mEF in fresh medium. Growing colonies having undifferentiated morphology can be individually selected by micropipette, mechanically dissociated into clumps, and replated. ES-like morphology is characterized as compact colonies with apparently high nucleus to cytoplasm ratio and prominent nucleoli. Resulting hESCs can then be routinely split every 1-2 weeks, for example, by brief trypsinization, exposure to Dulbecco's PBS (containing 2 mM EDTA), exposure to type IV collagenase (about 200 U/mL; Gibco) or by selection of individual colonies by micropipette. In some examples, clump sizes of about 50 to 100 cells are optimal. mESCs cells can be prepared from using the techniques described by e.g., Conner et al. (Curr. Prot. in Mol. Biol. Unit 23.4, 2003).

Embryonic stem cells can be isolated from blastocysts of members of the primate species (U.S. Pat. No. 5,843,780; Thomson et al., Proc. Natl. Acad. Sci. USA 92:7844, 1995). Human embryonic stem (hES) cells can be prepared from human blastocyst cells using the techniques described by Thomson et al. (U.S. Pat. No. 6,200,806; Science 282:1145, 1998; Curr. Top. Dev. Biol. 38:133 ff., 1998) and Reubinoff et al, Nature Biotech. 18:399, 2000. Equivalent cell types to hES cells include their pluripotent derivatives, such as primitive ectoderm-like (EPL) cells, as outlined in WO 01/51610 (Bresagen).

Alternatively, in some embodiments, hES cells can be obtained from human preimplantation embryos. Alternatively, in vitro fertilized (IVF) embryos can be used, or one-cell human embryos can be expanded to the blastocyst stage (Bongso et al., Hum Reprod 4: 706, 1989). Embryos are cultured to the blastocyst stage in G1.2 and G2.2 medium (Gardner et al., Fertil. Steril. 69:84, 1998). The zona pellucida is removed from developed blastocysts by brief exposure to pronase (Sigma). The inner cell masses are isolated by immunosurgery, in which blastocysts are exposed to a 1:50 dilution of rabbit anti-human spleen cell antiserum for 30 min, then washed for 5 min three times in DMEM, and exposed to a 1:5 dilution of Guinea pig complement (Gibco) for 3 min (Solter et al., Proc. Natl. Acad. Sci. USA 72:5099, 1975). After two further washes in DMEM, lysed trophectoderm cells are removed from the intact inner cell mass (ICM) by gentle pipetting, and the ICM plated on mEF feeder layers.

After 9 to 15 days, inner cell mass-derived outgrowths are dissociated into clumps, either by exposure to calcium and magnesium-free phosphate-buffered saline (PBS) with 1 mM EDTA, by exposure to dispase or trypsin, or by mechanical dissociation with a micropipette; and then replated on mEF in fresh medium. Growing colonies having undifferentiated morphology are individually selected by micropipette, mechanically dissociated into clumps, and replated. ES-like morphology is characterized as compact colonies with apparently high nucleus to cytoplasm ratio and prominent nucleoli. Resulting ES cells are then routinely split every 1-2 weeks by brief trypsinization, exposure to Dulbecco's PBS (containing 2 mM EDTA), exposure to type IV collagenase (^{˜}200 U/mL; Gibco) or by selection of individual colonies by micropipette. Clump sizes of about 50 to 100 cells are optimal.

In some embodiments, human Embryonic Germ (hEG) cells are pluripotent stem cells which can be used in the methods as disclosed herein to differentiate into primitive endoderm cells. hEG cells can be prepared from primordial germ cells present in human fetal material taken about 8-11 weeks after the last menstrual period. Suitable preparation methods are described in Shamblott et al., Proc. Natl. Acad. Sci. USA 95:13726, 1998 and U.S. Pat. No. 6,090,622, which is incorporated herein in its entirety by reference.

Briefly, genital ridges are processed to form disaggregated cells. EG growth medium is DMEM, 4500 mg/L D-glucose, 2200 mg/L mM NaHCO₃; 15% ES qualified fetal calf serum (BRL); 2 mM glutamine (BRL); 1 mM sodium pyruvate (BRL); 1000-2000 U/mL human recombinant leukemia inhibitory factor (LIF, Genzyme); 1-2 ng/mL human recombinant bFGF (Genzyme); and 10 μM forskolin (in 10% DMSO). Ninety-six well tissue culture plates are prepared with a sub-confluent layer of feeder cells (e.g., STO cells, ATCC No. CRL 1503) cultured for 3 days in modified EG growth medium free of LIF, bFGF or forskolin, inactivated with 5000 rad γ-irradiation ˜0.2 mL of primary germ cell (PGC) suspension is added to each of the wells. The first passage is done after 7-10 days in EG growth medium, transferring each well to one well of a 24-well culture dish previously prepared with irradiated STO mouse fibroblasts. The cells are cultured with daily replacement of medium until cell morphology consistent with EG cells is observed, typically after 7-30 days or 1-4 passages.

In certain examples, the stem cells can be undifferentiated (e.g. a cell not committed to a specific lineage) prior to exposure to at least one maturation factor according to the methods as disclosed herein, whereas in other examples it may be desirable to differentiate the stem cells to one or more intermediate cell types prior to exposure of the at least one maturation factor (s) described herein. For example, the stem cells may display morphological, biological or physical characteristics of undifferentiated cells that can be used to distinguish them from differentiated cells of embryo or adult origin. In some examples, undifferentiated cells may appear in the two dimensions of a microscopic view in colonies of cells with high nuclear/cytoplasmic ratios and prominent nucleoli. The stem cells may be themselves (for example, without substantially any undifferentiated cells being present) or may be used in the presence of differentiated cells. In certain examples, the stem cells may be cultured in the presence of suitable nutrients and optionally other cells such that the stem cells can grow and optionally differentiate. For example, embryonic fibroblasts or fibroblast-like cells may be present in the culture to assist in the growth of the stem cells. The fibroblast may be present during one stage of stem cell growth but not necessarily at all stages. For example, the fibroblast may be added to stem cell cultures in a first culturing stage and not added to the stem cell cultures in one or more subsequent culturing stages.

Stem cells used in all aspects of the present invention can be any cells derived from any kind of tissue (for example embryonic tissue such as fetal or pre-fetal tissue, or adult tissue), which stem cells have the characteristic of being capable under appropriate conditions of producing progeny of different cell types, e.g. derivatives of all of at least one of the 3 germinal layers (endoderm, mesoderm, and ectoderm). These cell types may be provided in the form of an established cell line, or they may be obtained directly from primary embryonic tissue and used immediately for differentiation. Included are cells listed in the NIH Human Embryonic Stem Cell Registry, e.g. hESBGN-01, hESBGN-02, hESBGN-03, hESBGN-04 (BresaGen, Inc.); HES-1, HES-2, HES-3, HES-4, HES-5, HES-6 (ES Cell International); Miz-hESI (MizMedi Hospital-Seoul National University); HSF-1, HSF-6 (University of California at San Francisco); and H1, H7, H9, H13, H14 (Wisconsin Alumni Research Foundation (WiCell Research Institute)). In some embodiments, the source of human stem cells or pluripotent stem cells used for chemically-induced differentiation into stem cell-derived cells did not involve destroying a human embryo.

In another embodiment, the stem cells can be isolated from tissue including solid tissue. In some embodiments, the tissue is skin, fat tissue (e.g. adipose tissue), muscle tissue, heart or cardiac tissue. In other embodiments, the tissue is for example but not limited to, umbilical cord blood, placenta, bone marrow, or chondral.

Stem cells of interest also include embryonic cells of various types, exemplified by human embryonic stem (hES) cells, described by Thomson et al. (1998) Science 282:1145; embryonic stem cells from other primates, such as Rhesus stem cells (Thomson et al. (1995) Proc. Natl. Acad. Sci. USA 92:7844); marmoset stem cells (Thomson et al. (1996) Biol. Reprod. 55:254); and human embryonic germ (hEG) cells (Shambloft et al., Proc. Natl. Acad. Sci. USA 95:13726, 1998). Also of interest are lineage committed stem cells, such as mesodermal stem cells and other early cardiogenic cells (see Reyes et al. (2001) Blood 98:2615-2625; Eisenberg & Bader (1996) Circ Res. 78(2):205-16; etc.) The stem cells may be obtained from any mammalian species, e.g. human, equine, bovine, porcine, canine, feline, rodent, e.g. mice, rats, hamster, primate, etc. In some embodiments, a human embryo was not destroyed for the source of pluripotent cell used on the methods and compositions as disclosed herein.

ES cells are considered to be undifferentiated when they have not committed to a specific differentiation lineage. Such cells display morphological characteristics that distinguish them from differentiated cells of embryo or adult origin. Undifferentiated ES cells are easily recognized by those skilled in the art, and typically appear in the two dimensions of a microscopic view in colonies of cells with high nuclear/cytoplasmic ratios and prominent nucleoli. Undifferentiated ES cells express genes that may be used as markers to detect the presence of undifferentiated cells, and whose polypeptide products may be used as markers for negative selection. For example, see U.S. application Ser. No. 2003/0224411 A1; Bhattacharya (2004) Blood 103(8):2956-64; and Thomson (1998), supra., each herein incorporated by reference. Human ES cell lines express cell surface markers that characterize undifferentiated nonhuman primate ES and human EC cells, including stage-specific embryonic antigen (SSEA)-3, SSEA-4, TRA-1-60, TRA-1-81, and alkaline phosphatase. The globo-series glycolipid GL7, which carries the SSEA-4 epitope, is formed by the addition of sialic acid to the globo-series glycolipid GbS, which carries the SSEA-3 epitope. Thus, GL7 reacts with antibodies to both SSEA-3 and SSEA-4. The undifferentiated human ES cell lines did not stain for SSEA-1, but differentiated cells stained strongly for SSEA-I. Methods for proliferating hES cells in the undifferentiated form are described in WO 99/20741, WO 01/51616, and WO 03/020920.

A mixture of cells from a suitable source of endothelial, muscle, and/or neural stem cells can be harvested from a mammalian donor by methods known in the art. A suitable source is the hematopoietic microenvironment. For example, circulating peripheral blood, preferably mobilized (i.e., recruited), may be removed from a subject. Alternatively, bone marrow may be obtained from a mammal, such as a human patient, undergoing an autologous transplant. In some embodiments, stem cells can be obtained from the subjects adipose tissue, for example using the CELUTION™ SYSTEM from Cytori, as disclosed in U.S. Pat. Nos. 7,390,484 and 7,429,488 which is incorporated herein in its entirety by reference.

In some embodiments, human umbilical cord blood cells (HUCBC) are useful in the methods as disclosed herein. Human UBC cells are recognized as a rich source of hematopoietic and mesenchymal progenitor cells (Broxmeyer et al., 1992 Proc. Natl. Acad. Sci. USA 89:4109-4113). Previously, umbilical cord and placental blood were considered a waste product normally discarded at the birth of an infant. Cord blood cells are used as a source of transplantable stem and progenitor cells and as a source of marrow repopulating cells for the treatment of malignant diseases (i.e. acute lymphoid leukemia, acute myeloid leukemia, chronic myeloid leukemia, myelodysplastic syndrome, and neuroblastoma) and non-malignant diseases such as Fanconi's anemia and aplastic anemia (Kohli-Kumar et al., 1993 Br. J. Haematol. 85:419-422; Wagner et al., 1992 Blood 79; 1874-1881; Lu et al., 1996 Crit. Rev. Oncol. Hematol 22:61-78; Lu et al., 1995 Cell Transplantation 4:493-503). A distinct advantage of HUCBC is the immature immunity of these cells that is very similar to fetal cells, which significantly reduces the risk for rejection by the host (Taylor & Bryson, 1985J. Immunol. 134:1493-1497). Human umbilical cord blood contains mesenchymal and hematopoietic progenitor cells, and endothelial cell precursors that can be expanded in tissue culture (Broxmeyer et al., 1992 Proc. Natl. Acad. Sci. USA 89:4109-4113; Kohli-Kumar et al., 1993 Br. J. Haematol. 85:419-422; Wagner et al., 1992 Blood 79; 1874-1881; Lu et al., 1996 Crit. Rev. Oncol. Hematol 22:61-78; Lu et al., 1995 Cell Transplantation 4:493-503; Taylor & Bryson, 1985J. Immunol. 134:1493-1497 Broxmeyer, 1995 Transfusion 35:694-702; Chen et al., 2001 Stroke 32:2682-2688; Nieda et al., 1997 Br. J. Haematology 98:775-777; Erices et al., 2000 Br. J. Haematology 109:235-242). The total content of hematopoietic progenitor cells in umbilical cord blood equals or exceeds bone marrow, and in addition, the highly proliferative hematopoietic cells are eightfold higher in HUCBC than in bone marrow and express hematopoietic markers such as CD14, CD34, and CD45 (Sanchez-Ramos et al., 2001 Exp. Neur. 171:109-115; Bicknese et al., 2002 Cell Transplantation 11:261-264; Lu et al., 1993 J. Exp Med. 178:2089-2096).

In another embodiment, pluripotent cells are cells in the hematopoietic micro-environment, such as the circulating peripheral blood, preferably from the mononuclear fraction of peripheral blood, umbilical cord blood, bone marrow, fetal liver, or yolk sac of a mammal. The stem cells, especially neural stem cells, may also be derived from the central nervous system, including the meninges.

In another embodiment, pluripotent cells are present in embryoid bodies are formed by harvesting ES cells with brief protease digestion and allowing small clumps of undifferentiated human ESCs to grow in suspension culture. Differentiation is induced by withdrawal of conditioned medium. The resulting embryoid bodies are plated onto semi-solid substrates. Formation of differentiated cells may be observed after around about 7 days to around about 4 weeks. Viable differentiating cells from in vitro cultures of stem cells are selected for by partially dissociating embryoid bodies or similar structures to provide cell aggregates. Aggregates comprising cells of interest are selected for phenotypic features using methods that substantially maintain the cell to cell contacts in the aggregate.

In an alternative embodiment, the stem cells can be reprogrammed stem cells, such as stem cells derived from somatic or differentiated cells. In such an embodiment, the de-differentiated stem cells can be for example, but not limited to, neoplastic cells, tumor cells and cancer cells or alternatively induced reprogrammed cells such as induced pluripotent stem cells or iPS cells.

### Cloning and Cell Culture

Illustrative methods for molecular genetics and genetic engineering that may be used in the technology described herein may be found, for example, in current editions of Molecular Cloning: A Laboratory Manual, (Sambrook et al., Cold Spring Harbor); Gene Transfer Vectors for Mammalian Cells (Miller & Calos eds.); and Current Protocols in Molecular Biology (F. M. Ausubel et al. eds., Wiley & Sons). Cell biology, protein chemistry, and antibody techniques can be found, for example, in Current Protocols in Protein Science (J. E. Colligan et al. eds., Wiley & Sons); Current Protocols in Cell Biology (J. S. Bonifacino et al., Wiley & Sons) and Current protocols in Immunology (J. E. Colligan et al. eds., Wiley & Sons.). Illustrative reagents, cloning vectors, and kits for genetic manipulation may be commercially obtained, for example, from BioRad, Stratagene, Invitrogen, ClonTech, and Sigma-Aldrich Co.

Suitable cell culture methods may be found, for example, in Cell culture methods are described generally in the current edition of Culture of Animal Cells: A Manual of Basic Technique (R. I. Freshney ed., Wiley & Sons); General Techniques of Cell Culture (M. A. Harrison & I. F. Rae, Cambridge Univ. Press), and Embryonic Stem Cells: Methods and Protocols (K. Turksen ed., Humana Press). Suitable tissue culture supplies and reagents are commercially available, for example, from Gibco/BRL, Nalgene-Nunc International, Sigma Chemical Co., and ICN Biomedicals.

Pluripotent stem cells can be propagated by one of ordinary skill in the art and continuously in culture, using culture conditions that promote proliferation without promoting differentiation. Exemplary serum-containing ES medium is made with 80% DMEM (such as Knock-Out DMEM, Gibco), 20% of either defined fetal bovine serum (FBS, Hyclone) or serum replacement (WO 98/30679), 1% non-essential amino acids, 1 mM L-glutamine, and 0.1 mM P-mercaptoethanol. Just before use, human bFGF is added to 4 ng/mL (WO 99/20741, Geron Corp.). Traditionally, ES cells are cultured on a layer of feeder cells, typically fibroblasts derived from embryonic or fetal tissue.

Scientists at Geron have discovered that pluripotent SCs can be maintained in an undifferentiated state even without feeder cells. The environment for feeder-free cultures includes a suitable culture substrate, particularly an extracellular matrix such as Matrigel® or laminin. Typically, enzymatic digestion is halted before cells become completely dispersed (say, ^{˜}5 min with collagenase IV). Clumps of ^{˜}10 to 2,000 cells are then plated directly onto the substrate without further dispersal.

Feeder-free cultures are supported by a nutrient medium containing factors that support proliferation of the cells without differentiation. Such factors may be introduced into the medium by culturing the medium with cells secreting such factors, such as irradiated (^{˜}4,000 rad) primary mouse embryonic fibroblasts, telomerized mouse fibroblasts, or fibroblast-like cells derived from pPS cells. Medium can be conditioned by plating the feeders at a density of ^{˜}5-6×10⁴ cm⁻² in a serum free medium such as KO DMEM supplemented with 20% serum replacement and 4 ng/mL bFGF. Medium that has been conditioned for 1-2 days is supplemented with further bFGF and used to support pluripotent SC culture for 1-2 days. Features of the feeder-free culture method are further discussed in International Patent Publication WO 01/51616; and Xu et al., Nat. Biotechnol. 19:971, 2001.

Under the microscope, ES cells appear with high nuclear/cytoplasmic ratios, prominent nucleoli, and compact colony formation with poorly discernable cell junctions. Primate ES cells express stage-specific embryonic antigens (SSEA) 3 and 4, and markers detectable using antibodies designated Tra-1-60 and Tra-1-81 (Thomson et al., Science 282:1145, 1998). Mouse ES cells can be used as a positive control for SSEA-1, and as a negative control for SSEA-4, Tra-1-60, and Tra-1-81. SSEA-4 is consistently present human embryonal carcinoma (hEC) cells. Differentiation of pluripotent SCs in vitro results in the loss of SSEA-4, Tra-1-60, and Tra-1-81 expression, and increased expression of SSEA-1, which is also found on undifferentiated hEG cells.

### Methods of Generating Stem Cell-Derived Cells

Aspects of the disclosure relate to generating stem cell-derived cells (e.g., SC-β cells, SC-EC cells, SC-α cells, etc.). Generally, the at least one stem cell-derived cell or precursor thereof, e.g., pancreatic progenitors produced according to the methods disclosed herein, can comprise a mixture or combination of different cells, e.g., for example a mixture of cells such as a Pdx1+ pancreatic progenitors, pancreatic progenitors co-expressing Pdx1 and NKX6-1, Ngn3-positive endocrine progenitors, endocrine cells (e.g., 3-like cells, a-like cells, EC-like cells), non-endocrine cells, and/or other pluripotent or stem cells.

In some embodiments, a somatic cell, e.g., fibroblast can be isolated from a subject, for example as a tissue biopsy, such as, for example, a skin biopsy, and reprogrammed into an induced pluripotent stem cell for further differentiation to produce the at least one stem cell-derived cell or precursor thereof for use in the compositions and methods described herein. In some embodiments, a somatic cell, e.g., fibroblast is maintained in culture by methods known by one of ordinary skill in the art, and in some embodiments, propagated prior to being converted into stem cell-derived cells by the methods as disclosed herein.

In some embodiments, a progenitor cell is genetically modified prior to being converted into a stem cell-derived cell by the methods as disclosed herein. In some embodiments, a progenitor cell is genetically modified to inhibit or knock out an essential factor or regulator thereby inhibiting development of a specific cell type (e.g., SC-EC cells and TN cells). In some embodiments, a stem cell is genetically modified to inhibit or knock out an essential factor or regulator thereby increasing development of a specific cell type (e.g., SC-β cells or SC-α cells). In one embodiment, a stem cell is genetically modified to inhibit or knock out an essential factor or regulator thereby increasing development of an SC-β cell. In one embodiment, a stem cell is genetically modified to inhibit or knock out an essential factor or regulator thereby increasing development of an SC-α cell. In one embodiment, a stem cell is genetically modified to inhibit or knock out an essential factor or regulator thereby increasing development of an SC-EC cell. In one embodiment, a stem cell is genetically modified to inhibit or knock out an essential factor or regulator thereby decreasing development of an SC-EC cell. In some embodiments the targeting of the essential factor or regulator occurs using any gene editing tool known to those of skill in the art (e.g., TALENS, CRISPR, etc.). In some embodiments, the gene editing tool is delivered to the stem cells using a retrovirus (e.g., a lentivirus).

In some embodiments, one or more genes may be identified as controlling cell fate during a differentiation protocol. In some embodiments, the one or more genes may be targeted using gene editing (e.g., CRISPR) to modulate expression of the one or more genes and thereby control the fate of the differentiation process. In some aspects a first gene may be knocked out or inhibited in a progenitor cell prior to the progenitor cell being converted into a stem cell-derived cell. In some aspects, a first gene and a second gene are knocked out or inhibited in a progenitor cell prior to the progenitor cell being converted into a stem cell-derived cell.

In some aspects, differentiation of a population of progenitor cells is directed towards a SC-β cell fate by knocking down or knocking out expression of one or more genes listed in Table 1. The knocking down or knocking out expression of the one or more genes listed in Table 1 in a progenitor cell may direct cell differentiation of the progenitor cell towards a SC-β cell and away from a triple negative cell. In some embodiments, the one or more genes are selected from the group consisting of FBXL14, BCORL1, SHOC2, CCDC6, B3GALT6, HOXA1, DDX3X, CARM1, EXT2, EXT1, DYRK1A, SCAF1, SCAF8, CAND1, NDST1, EYA3, GLCE, DYRK1B, PRDM16, ALG3, CXXC4, SMURF1, PHF21A, SOX4, and TET2. In some embodiments, the one or more genes are selected from the group consisting of FBXL14, BCORL1, SHOC2, CCDC6, B3GALT6, HOXA1, DDX3X, CARM1, EXT2, and EXT1. In some aspects, differentiation of a population of progenitor cells is directed towards a SC-β cell fate by knocking down or knocking out expression of one or more genes listed in Table 2. The knocking down or knocking out expression of the one or more genes listed in Table 2 in a progenitor cell may direct cell differentiation of the progenitor cell towards a SC-β cell and away from an SC-EC cell. In some embodiments, the one or more genes are selected from the group consisting of SOX4, BCORL1, FBXL14, CCDC6, SOX1, CARM1, TNRC18, CAND1, TET2, HOXA1, ASCL1, ARID2, SIRT6, FBXO22, FLVCR1, FOXA1, COPS9, ELAVL1, SSBP3, PROSER1, PROX1, SMURF1, SCAF1, HELLS, and DACH1. In certain embodiments, the one or more genes are selected from the group consisting of SOX4, BCORL1, FBXL14, CCDC6, SOX1, CARM1, TNRC18, CAND1, TET2, and HOXA1.

TABLE 1
Top 50 genes most pro-beta relative to
TN (TN = triple negative; non endocrine)

| | score of | score of | score of | score of | score of |
|---|---|---|---|---|---|
| | SC-beta | SC-alpha | SC-EC | SC-beta | SC-alpha |
| | relative | relative | relative | relative | relative |
| Column1 | to TN | to TN | to TN | to SC-EC | to SC-EC |
| FBXL14 | 1.04 | 0.26 | 0.29 | 0.75 | −0.03 |
| BCORL1 | 0.71 | 0.37 | −0.13 | 0.84 | 0.5 |
| SHOC2 | 0.56 | 0.11 | 0.58 | −0.02 | −0.47 |
| CCDC6 | 0.53 | 0.73 | −0.02 | 0.55 | 0.75 |
| B3GALT6 | 0.48 | 0.21 | 0.3 | 0.18 | −0.1 |
| HOXA1 | 0.48 | 0.26 | 0.11 | 0.37 | 0.14 |
| DDX3X | 0.38 | 0.33 | 0.32 | 0.06 | 0.01 |
| CARM1 | 0.37 | −0.62 | −0.1 | 0.46 | −0.52 |
| EXT2 | 0.36 | 0.16 | 0.24 | 0.12 | −0.08 |
| EXT1 | 0.36 | 0.16 | 0.28 | 0.07 | −0.13 |
| DYRK1A | 0.32 | 0.47 | 0.16 | 0.17 | 0.31 |
| SCAF1 | 0.3 | −0.07 | 0.08 | 0.22 | −0.15 |
| SCAF8 | 0.29 | 0.24 | 0.28 | 0.01 | −0.04 |
| CAND1 | 0.29 | 0.3 | −0.1 | 0.4 | 0.41 |
| NDST1 | 0.29 | 0.19 | 0.09 | 0.2 | 0.09 |
| EYA3 | 0.27 | 0.3 | 0.19 | 0.08 | 0.1 |
| GLCE | 0.26 | 0.09 | 0.11 | 0.14 | −0.02 |
| DYRK1B | 0.25 | 0.47 | 0.13 | 0.12 | 0.34 |
| PRDM16 | 0.25 | 0.21 | 0.11 | 0.14 | 0.1 |
| ALG3 | 0.23 | 0.19 | 0.22 | 0 | −0.04 |
| CXXC4 | 0.21 | −0.12 | 0.09 | 0.13 | −0.21 |
| SMURF1 | 0.2 | 0.15 | −0.03 | 0.23 | 0.18 |
| PHF21A | 0.2 | 0.64 | 0 | 0.2 | 0.64 |
| SOX4 | 0.2 | −0.13 | −0.79 | 0.99 | 0.66 |
| TET2 | 0.19 | 0.34 | −0.18 | 0.37 | 0.52 |
| HEXIM1 | 0.19 | 0.21 | 0.12 | 0.07 | 0.09 |
| COPS9 | 0.18 | −0.28 | −0.09 | 0.28 | −0.19 |
| SIX4 | 0.18 | 0.16 | 0.11 | 0.07 | 0.06 |
| PROSER1 | 0.18 | 0.33 | −0.07 | 0.24 | 0.39 |
| HELLS | 0.18 | 0.3 | −0.04 | 0.21 | 0.34 |
| ZC3H15 | 0.18 | 0.25 | −0.02 | 0.2 | 0.28 |
| SSBP3 | 0.17 | −0.4 | −0.08 | 0.26 | −0.32 |
| ELAVL1 | 0.17 | −0.28 | −0.09 | 0.26 | −0.19 |
| DCUN1D3 | 0.17 | 0.14 | 0.04 | 0.12 | 0.1 |
| PHC3 | 0.15 | 0.15 | 0.07 | 0.08 | 0.08 |
| RNF7 | 0.15 | 0.07 | 0.11 | 0.05 | −0.04 |
| ZBTB7B | 0.15 | −0.16 | −0.02 | 0.17 | −0.15 |
| DRG1 | 0.14 | 0.2 | −0.05 | 0.19 | 0.24 |
| MYT1 | 0.14 | 0.29 | 0.03 | 0.11 | 0.26 |
| ARID5B | 0.14 | 0.02 | −0.04 | 0.18 | 0.06 |
| ZNF384 | 0.14 | 0.08 | 0.07 | 0.07 | 0.01 |
| TOMM7 | 0.13 | 0.03 | 0.11 | 0.02 | −0.08 |
| TYW5 | 0.13 | 0.14 | 0.05 | 0.07 | 0.09 |
| SENP1 | 0.13 | 0.13 | −0.01 | 0.14 | 0.14 |
| MARCH6 | 0.13 | 0.21 | 0.11 | 0.02 | 0.1 |
| HNRNPD | 0.12 | −0.14 | 0.09 | 0.03 | −0.24 |
| PARD6B | 0.12 | 0.13 | 0.07 | 0.05 | 0.06 |
| CD84 | 0.12 | 0.05 | 0.09 | 0.03 | −0.04 |
| SIX2 | 0.12 | 0.11 | −0.06 | 0.18 | 0.18 |
| LMX1A | 0.11 | 0.05 | −0.05 | 0.16 | 0.1 |

TABLE 2
Top 50 genes most pro-beta relative to EC (enterochromaffin)

| | score of | score of | score of | score of | score of |
|---|---|---|---|---|---|
| | SC-beta | SC-alpha | SC-EC | SC-beta | SC-alpha |
| | relative | relative | relative | relative | relative |
| Column1 | to TN | to TN | to TN | to SC-EC | to SC-EC |
| SOX4 | 0.2 | −0.13 | −0.79 | 0.99 | 0.66 |
| BCORL1 | 0.71 | 0.37 | −0.13 | 0.84 | 0.5 |
| FBXL14 | 1.04 | 0.26 | 0.29 | 0.75 | −0.03 |
| CCDC6 | 0.53 | 0.73 | −0.02 | 0.55 | 0.75 |
| SOX1 | 0.1 | −0.02 | −0.39 | 0.49 | 0.37 |
| CARM1 | 0.37 | −0.62 | −0.1 | 0.46 | −0.52 |
| TNRC18 | 0.03 | −0.19 | −0.38 | 0.41 | 0.19 |
| CAND1 | 0.29 | 0.3 | −0.1 | 0.4 | 0.41 |
| TET2 | 0.19 | 0.34 | −0.18 | 0.37 | 0.52 |
| HOXA1 | 0.48 | 0.26 | 0.11 | 0.37 | 0.14 |
| ASCL1 | −0.05 | −0.1 | −0.4 | 0.34 | 0.29 |
| ARID2 | −0.17 | 0.03 | −0.5 | 0.33 | 0.54 |
| SIRT6 | −0.15 | −0.39 | −0.48 | 0.33 | 0.09 |
| FBXO22 | 0.01 | 0.07 | −0.29 | 0.31 | 0.37 |
| FLVCR1 | 0.11 | −0.28 | −0.17 | 0.28 | −0.11 |
| FOXA1 | −0.15 | −0.41 | −0.43 | 0.28 | 0.02 |
| COPS9 | 0.18 | −0.28 | −0.09 | 0.28 | −0.19 |
| ELAVL1 | 0.17 | −0.28 | −0.09 | 0.26 | −0.19 |
| SSBP3 | 0.17 | −0.4 | −0.08 | 0.26 | −0.32 |
| PROSER1 | 0.18 | 0.33 | −0.07 | 0.24 | 0.39 |
| PROX1 | −0.01 | −0.54 | −0.25 | 0.24 | −0.29 |
| SMURF1 | 0.2 | 0.15 | −0.03 | 0.23 | 0.18 |
| SCAF1 | 0.3 | −0.07 | 0.08 | 0.22 | −0.15 |
| HELLS | 0.18 | 0.3 | −0.04 | 0.21 | 0.34 |
| DACH1 | 0.1 | −0.09 | −0.1 | 0.2 | 0.01 |
| PHF21A | 0.2 | 0.64 | 0 | 0.2 | 0.64 |
| NDST1 | 0.29 | 0.19 | 0.09 | 0.2 | 0.09 |
| ZC3H15 | 0.18 | 0.25 | −0.02 | 0.2 | 0.28 |
| H3F3B | 0.05 | 0.1 | −0.14 | 0.19 | 0.24 |
| ZRANB1 | 0.08 | 0.21 | −0.1 | 0.19 | 0.31 |
| DRG1 | 0.14 | 0.2 | −0.05 | 0.19 | 0.24 |
| SIX2 | 0.12 | 0.11 | −0.06 | 0.18 | 0.18 |
| ARID5B | 0.14 | 0.02 | −0.04 | 0.18 | 0.06 |
| B3GALT6 | 0.48 | 0.21 | 0.3 | 0.18 | −0.1 |
| SOX2 | 0.09 | 0.07 | −0.08 | 0.17 | 0.15 |
| ZBTB7B | 0.15 | −0.16 | −0.02 | 0.17 | −0.15 |
| DYRK1A | 0.32 | 0.47 | 0.16 | 0.17 | 0.31 |
| JMJD1C | 0.1 | −0.15 | −0.07 | 0.16 | −0.08 |
| LMX1A | 0.11 | 0.05 | −0.05 | 0.16 | 0.1 |
| TTC14 | −0.04 | 0.21 | −0.2 | 0.16 | 0.41 |
| C16orf87 | 0.09 | 0.04 | −0.07 | 0.16 | 0.11 |
| CHAMP1 | −0.06 | −0.07 | −0.22 | 0.15 | 0.14 |
| TGIF2 | −0.07 | 0.03 | −0.22 | 0.15 | 0.25 |
| DHX29 | 0.1 | 0.09 | −0.04 | 0.15 | 0.13 |
| GLCE | 0.26 | 0.09 | 0.11 | 0.14 | −0.02 |
| PRDM16 | 0.25 | 0.21 | 0.11 | 0.14 | 0.1 |
| SENP1 | 0.13 | 0.13 | −0.01 | 0.14 | 0.14 |
| FAAP100 | 0.11 | 0.01 | −0.02 | 0.13 | 0.03 |
| TRIP12 | 0.03 | −0.76 | −0.09 | 0.13 | −0.66 |
| CXXC4 | 0.21 | −0.12 | 0.09 | 0.13 | −0.21 |

In some aspects, differentiation of a population of progenitor cells is directed towards a SC-α cell fate by knocking down or knocking out expression of one or more genes listed in Table 3. The knocking down or knocking out expression of the one or more genes listed in Table 3 in a progenitor cell may direct cell differentiation of the progenitor cell towards a SC-α cell and away from a triple negative cell. In some embodiments, the one or more genes are selected from the group consisting of PDX1, CCDC6, HES1, PHF21A, PAX4, DYRK1B, DYRK1A, BCORL1, TET2, DDX3X, PROSER1, PBX1, HELLS, CAND1, EYA3, MYT1, AFF4, FBXL14, HOXA1, ZC31H15, SCAF8, PRDM16, HEXIM1, TTC14, ZRANB1, and B3GALT6. In some embodiments, the one or more genes are selected from the group consisting of PDX1, CCDC6, HES1, PHF21A, PAX4, DYRK1B, DYRK1A, BCORL1, TET2, and DDX3X. In some aspects, differentiation of a population of a progenitor cells is directed towards a SC-α cell fate by knocking down or knocking out expression of one or more genes listed in Table 4. The knocking down or knocking out expression of the one or more genes listed in Table 4 in a progenitor cell may direct cell differentiation of the progenitor cell towards a SC-α cell and away from an SC-EC cell. In some embodiments, the one or more genes are selected from the group consisting of PAX4, HES, CCDC6, DCX4, ZBTB10, PHF21A, PBX1, ARID2, TET2, BCORL1, TTC14, CAND1, PROSER1, SOX1, FBXO22, HELLS, DYRK1B, ZRANB1, DYRK1A, ASCL1, ZC3H15, SETBP1, FAM58A, MYT1, and RALGAPB. In certain embodiments, the one or more genes are selected from the group consisting of PAX4, HES, CCDC6, SOX4, ZBTB10, PHF21A, PBX1, ARID2, TET2, and BCORL1.

TABLE 3
Top 50 genes most pro-alpha relative to
TN (TN = triple negative; non endocrine)

| | score of | score of | score of | score of | score of |
|---|---|---|---|---|---|
| | SC-beta | SC-alpha | SC-EC | SC-beta | SC-alpha |
| | relative | relative | relative | relative | relative |
| Column1 | to TN | to TN | to TN | to SC-EC | to SC-EC |
| PDX1 | −0.78 | 0.83 | 1.16 | −1.5 | −0.33 |
| CCDC6 | 0.53 | 0.73 | −0.02 | 0.55 | 0.75 |
| HES1 | −0.18 | 0.7 | −0.11 | −0.07 | 0.82 |
| PHF21A | 0.2 | 0.64 | 0 | 0.2 | 0.64 |
| PAX4 | −0.76 | 0.55 | −0.9 | 0.14 | 1.45 |
| DYRK1B | 0.25 | 0.47 | 0.13 | 0.12 | 0.34 |
| DYRK1A | 0.32 | 0.47 | 0.16 | 0.17 | 0.31 |
| BCORL1 | 0.71 | 0.37 | −0.13 | 0.84 | 0.5 |
| TET2 | 0.19 | 0.34 | −0.18 | 0.37 | 0.52 |
| DDX3X | 0.38 | 0.33 | 0.32 | 0.06 | 0.01 |
| PROSER1 | 0.18 | 0.33 | −0.07 | 0.24 | 0.39 |
| PBX1 | −0.16 | 0.32 | −0.25 | 0.1 | 0.58 |
| HELLS | 0.18 | 0.3 | −0.04 | 0.21 | 0.34 |
| CAND1 | 0.29 | 0.3 | −0.1 | 0.4 | 0.41 |
| EYA3 | 0.27 | 0.3 | 0.19 | 0.08 | 0.1 |
| MYT1 | 0.14 | 0.29 | 0.03 | 0.11 | 0.26 |
| AFF4 | 0.01 | 0.28 | 0.09 | −0.08 | 0.19 |
| FBXL14 | 1.04 | 0.26 | 0.29 | 0.75 | −0.03 |
| HOXA1 | 0.48 | 0.26 | 0.11 | 0.37 | 0.14 |
| ZC3H15 | 0.18 | 0.25 | −0.02 | 0.2 | 0.28 |
| SCAF8 | 0.29 | 0.24 | 0.28 | 0.01 | −0.04 |
| PRDM16 | 0.25 | 0.21 | 0.11 | 0.14 | 0.1 |
| HEXIM1 | 0.19 | 0.21 | 0.12 | 0.07 | 0.09 |
| TTC14 | −0.04 | 0.21 | −0.2 | 0.16 | 0.41 |
| ZRANB1 | 0.08 | 0.21 | −0.1 | 0.19 | 0.31 |
| B3GALT6 | 0.48 | 0.21 | 0.3 | 0.18 | −0.1 |
| MARCH6 | 0.13 | 0.21 | 0.11 | 0.02 | 0.1 |
| PTF1A | −0.15 | 0.2 | −0.03 | −0.12 | 0.23 |
| DRG1 | 0.14 | 0.2 | −0.05 | 0.19 | 0.24 |
| ALG3 | 0.23 | 0.19 | 0.22 | 0 | −0.04 |
| NDST1 | 0.29 | 0.19 | 0.09 | 0.2 | 0.09 |
| DCAF10 | 0 | 0.18 | 0.04 | −0.04 | 0.14 |
| UBQLN2 | 0.09 | 0.17 | 0.02 | 0.07 | 0.15 |
| C12orf66 | 0.08 | 0.17 | 0.09 | −0.01 | 0.08 |
| SIX4 | 0.18 | 0.16 | 0.11 | 0.07 | 0.06 |
| EXT2 | 0.36 | 0.16 | 0.24 | 0.12 | −0.08 |
| EXT1 | 0.36 | 0.16 | 0.28 | 0.07 | −0.13 |
| SMURF1 | 0.2 | 0.15 | −0.03 | 0.23 | 0.18 |
| PHC3 | 0.15 | 0.15 | 0.07 | 0.08 | 0.08 |
| ABI1 | 0.07 | 0.15 | 0.15 | −0.08 | 0 |
| CDC37L1 | 0.06 | 0.15 | −0.02 | 0.08 | 0.17 |
| DCUN1D3 | 0.17 | 0.14 | 0.04 | 0.12 | 0.1 |
| FNDC3A | 0.06 | 0.14 | 0.05 | 0 | 0.09 |
| TYW5 | 0.13 | 0.14 | 0.05 | 0.07 | 0.09 |
| SYF2 | 0.06 | 0.14 | 0.05 | 0.01 | 0.09 |
| MIDN | −0.11 | 0.13 | 0.16 | −0.27 | −0.03 |
| SENP1 | 0.13 | 0.13 | −0.01 | 0.14 | 0.14 |
| RAPGEF6 | 0.09 | 0.13 | 0.04 | 0.06 | 0.09 |
| LIMD1 | 0.1 | 0.13 | 0.05 | 0.05 | 0.08 |
| PARD6B | 0.12 | 0.13 | 0.07 | 0.05 | 0.06 |

TABLE 4
Group 4: Top 50 genes most pro-alpha
relative to EC (enterochromaffin)

| | score of | score of | score of | score of | score of |
|---|---|---|---|---|---|
| | SC-beta | SC-alpha | SC-EC | SC-beta | SC-alpha |
| | relative | relative | relative | relative | relative |
| Column1 | to TN | to TN | to TN | to SC-EC | to SC-EC |
| PAX4 | −0.76 | 0.55 | −0.9 | 0.14 | 1.45 |
| HES1 | −0.18 | 0.7 | −0.11 | −0.07 | 0.82 |
| CCDC6 | 0.53 | 0.73 | −0.02 | 0.55 | 0.75 |
| SOX4 | 0.2 | −0.13 | −0.79 | 0.99 | 0.66 |
| ZBTB10 | −0.27 | −0.01 | −0.65 | 0.38 | 0.64 |
| PHF21A | 0.2 | 0.64 | 0 | 0.2 | 0.64 |
| PBX1 | −0.16 | 0.32 | −0.25 | 0.1 | 0.58 |
| ARID2 | −0.17 | 0.03 | −0.5 | 0.33 | 0.54 |
| TET2 | 0.19 | 0.34 | −0.18 | 0.37 | 0.52 |
| BCORL1 | 0.71 | 0.37 | −0.13 | 0.84 | 0.5 |
| TTC14 | −0.04 | 0.21 | −0.2 | 0.16 | 0.41 |
| CAND1 | 0.29 | 0.3 | −0.1 | 0.4 | 0.41 |
| PROSER1 | 0.18 | 0.33 | −0.07 | 0.24 | 0.39 |
| SOX1 | 0.1 | −0.02 | −0.39 | 0.49 | 0.37 |
| FBXO22 | 0.01 | 0.07 | −0.29 | 0.31 | 0.37 |
| HELLS | 0.18 | 0.3 | −0.04 | 0.21 | 0.34 |
| DYRK1B | 0.25 | 0.47 | 0.13 | 0.12 | 0.34 |
| ZRANB1 | 0.08 | 0.21 | −0.1 | 0.19 | 0.31 |
| DYRK1A | 0.32 | 0.47 | 0.16 | 0.17 | 0.31 |
| ASCL1 | −0.05 | −0.1 | −0.4 | 0.34 | 0.29 |
| ZC3H15 | 0.18 | 0.25 | −0.02 | 0.2 | 0.28 |
| SETBP1 | −0.2 | 0.02 | −0.25 | 0.05 | 0.27 |
| FAM58A | −0.05 | 0.11 | −0.16 | 0.11 | 0.27 |
| MYT1 | 0.14 | 0.29 | 0.03 | 0.11 | 0.26 |
| RALGAPB | −0.56 | −0.19 | −0.44 | −0.11 | 0.25 |
| TGIF2 | −0.07 | 0.03 | −0.22 | 0.15 | 0.25 |
| DRG1 | 0.14 | 0.2 | −0.05 | 0.19 | 0.24 |
| H3F3B | 0.05 | 0.1 | −0.14 | 0.19 | 0.24 |
| PTF1A | −0.15 | 0.2 | −0.03 | −0.12 | 0.23 |
| ZNF292 | −0.23 | 0.03 | −0.17 | −0.06 | 0.2 |
| AFF4 | 0.01 | 0.28 | 0.09 | −0.08 | 0.19 |
| TNRC18 | 0.03 | −0.19 | −0.38 | 0.41 | 0.19 |
| SMURF1 | 0.2 | 0.15 | −0.03 | 0.23 | 0.18 |
| SIX2 | 0.12 | 0.11 | −0.06 | 0.18 | 0.18 |
| CDC37L1 | 0.06 | 0.15 | −0.02 | 0.08 | 0.17 |
| UBQLN2 | 0.09 | 0.17 | 0.02 | 0.07 | 0.15 |
| SOX2 | 0.09 | 0.07 | −0.08 | 0.17 | 0.15 |
| CHAMP1 | −0.06 | −0.07 | −0.22 | 0.15 | 0.14 |
| HOXA1 | 0.48 | 0.26 | 0.11 | 0.37 | 0.14 |
| DCAF10 | 0 | 0.18 | 0.04 | −0.04 | 0.14 |
| SENP1 | 0.13 | 0.13 | −0.01 | 0.14 | 0.14 |
| DHX29 | 0.1 | 0.09 | −0.04 | 0.15 | 0.13 |
| TRAF7 | −0.02 | 0.1 | −0.03 | 0.01 | 0.13 |
| SOX9 | −0.4 | 0.02 | −0.11 | −0.3 | 0.13 |
| CCND1 | 0.06 | 0.11 | −0.02 | 0.08 | 0.13 |
| THAP4 | 0.08 | 0.12 | 0 | 0.08 | 0.12 |
| CSNK2A1 | −0.09 | −0.01 | −0.12 | 0.04 | 0.12 |
| RAD23B | −0.04 | 0.11 | 0 | −0.04 | 0.12 |
| C16orf87 | 0.09 | 0.04 | −0.07 | 0.16 | 0.11 |
| DUS1L | 0.06 | 0.12 | 0 | 0.06 | 0.11 |

In some aspects, an increased population of SC-β cells is generated by inhibiting development of SC-EC cells. In some aspects, an increased population of SC-β cells is generated by inhibiting development of TN cells. In some aspects, an increased population of SC-α cells is generated by inhibiting development of SC-EC cells. In some aspects, an increased population of SC-α cells is generated by inhibiting development of TN cells. By disrupting SC-EC cell production and/or TN cell production during differentiation, the resulting population of differentiated cells will exhibit an increased yield of SC-β cells and/or SC-α cells. In some embodiments, overexpression of one or more transcription factors disrupts SC-EC formation. In some aspects, the overexpression of one or more transcription factors alters or changes endocrine cell ratios. In some embodiments, the overexpression of one or more transcription factors results in an increased population of SC-α cells and/or SC-β cells. In one embodiment, the overexpression of ISL1 results in an increase in SC-β cells and/or SC-α cells and reduces the formation of SC-EC cells.

In some embodiments, the at least one stem cell-derived cell or precursor thereof is maintained in culture by methods known by one of ordinary skill in the art, and in some embodiments, propagated prior to being converted into stem cell-derived cells by the methods as disclosed herein.

Further, at least one stem cell-derived cell or precursor thereof, e.g., pancreatic progenitor, can be from any mammalian species, with non-limiting examples including a murine, bovine, simian, porcine, equine, ovine, or human cell. For clarity and simplicity, the description of the methods herein refers to a mammalian at least one stem cell-derived cell or precursor thereof but it should be understood that all of the methods described herein can be readily applied to other cell types of at least one stem cell-derived cell or precursor thereof. In some embodiments, the at least one stem cell-derived cell or precursor thereof is derived from a human individual.

The at least one stem cell-derived cell or precursor thereof can be produced according to any suitable culturing protocol to differentiate a stem cell or pluripotent cell to a desired stage of differentiation. In some embodiments, the at least one stem cell-derived cell or the precursor thereof are produced by culturing at least one pluripotent cell for a period of time and under conditions suitable for the at least one pluripotent cell to differentiate into the at least one stem cell-derived cell or the precursor thereof.

In some embodiments, the at least one stem cell-derived cell or precursor thereof is a substantially pure population of stem cell-derived cells or precursors thereof. In some embodiments, a population of stem cell-derived cells or precursors thereof comprises a mixture of pluripotent cells or differentiated cells (e.g., a mixture of SC-β cells, SC-α cells, SC-EC cells, and/or other differentiated cell types, also referred to herein as triple negative cells or TN cells). In some embodiments, a population of SC-β cells or precursors thereof are substantially free or devoid of embryonic stem cells or pluripotent cells or iPS cells. In some embodiments, a population of SC-α cells or precursors thereof are substantially free or devoid of embryonic stem cells or pluripotent cells or iPS cells. In some embodiments, a population of SC-EC cells or precursors thereof are substantially free or devoid of embryonic stem cells or pluripotent cells or iPS cells.

In some embodiments stem cell-derived cells (e.g., pancreatic stem cell-derived cells) may be produced using methods known to those of skill in the art. In certain embodiments, stem cell-derived cells may be produced using the methods disclosed in WO 2015/002724, WO 2014/201167, WO 2019/217493, and/or WO 2019/217487, all of which are incorporated herein by reference.

### Transcriptional Profiling of Stages of a Differentiation Protocol

In some aspects of the disclosure, single-cell sequencing (e.g., high throughput single-cell RNA sequencing) is used to provide a detailed characterization of the full transcriptomes of all cell populations produced using an in vitro differentiation protocol (e.g., an in vitro beta cell or alpha cell differentiation protocol). In some embodiments, specific genes are identified as enriching a single population of cells or combination of cells. In some aspects single-cell sequencing is performed at all stages of an in vitro differentiation protocol. In some embodiments, sequencing is performed at the end of Stage 6 of a differentiation protocol (e.g., a beta cell or alpha cell differentiation protocol).

In some aspects of the disclosure, at the completion of the differentiation protocol (e.g., after Stage 6), clusters are formed. In some embodiments the clusters comprise one or more cell types. In some aspects the clusters are screened to identify the various cells included within the cluster. In some embodiments, the clusters are screened using single-cell sequencing (e.g., high throughput single-cell RNA sequencing) to identify the cells located with the clusters. In some aspects the clusters comprise one or more of SC-β cells, SC-α cells, SC-δ cells, SC-EC cells, and TN cells.

### Pancreatic Stem Cell-Derived Cells

In some aspects of the disclosure, stem cell-derived cells (e.g., pancreatic stem cell-derived cells) are provided. In some embodiments, the stem cell-derived cells are SC-β cells, SC-α cells, and/or SC-δ cells. The stem cell-derived cells disclosed herein share many distinguishing features of native pancreatic cells but are different in certain aspects. In some embodiments, the stem cell-derived cells are non-native, i.e., non-naturally occurring, non-endogenous cells. As used herein, “non-native” means that the stem cell-derived cells are markedly different in certain aspects from cells which exist in nature, i.e., native cells. It should be appreciated, however, that these marked differences may result in the stem cell-derived cells exhibiting certain differences, but the stem cell-derived cells may still behave in a similar manner to native cells with certain functions altered (e.g., improved) compared to the native cells.

The stem cell-derived cells are differentiated in vitro from any starting cell as the invention is not intended to be limited by the starting cell from which the stem cell-derived cells are derived. Exemplary starting cells include, without limitation, endocrine cells or any precursor thereof such as a NKX6-1+ pancreatic progenitor cell, a Pdx1+ pancreatic progenitor cell, and a pluripotent stem cell, an embryonic stem cell, and induced pluripotent stem cell. In some embodiments, the stem cell-derived cells are differentiated in vitro from a reprogrammed cell, a partially reprogrammed cell (i.e., a somatic cell, e.g., a fibroblast which has been partially reprogrammed such that it exists in an intermediate state between an induced pluripotency cell and the somatic cell from which it has been derived), a transdifferentiated cell. In some embodiments, the stem cell-derived cells disclosed herein can be differentiated in vitro from an endocrine cell or a precursor thereof. In some embodiments, the stem cell-derived cell is differentiated in vitro from a precursor selected from the group consisting of a NKX6-1+ pancreatic progenitor cell, a Pdx1+ pancreatic progenitor cell, and a pluripotent stem cell. In some embodiments, the pluripotent stem cell is selected from the group consisting of an embryonic stem cell and induced pluripotent stem cell. In some embodiments, the stem cell-derived cell or the pluripotent stem cell from which the stem cell-derived cell is derived is human. In some embodiments, the stem cell-derived cell is human.

In some embodiments, the stem cell-derived cell is not genetically modified. In some embodiments, the stem cell-derived cell obtains the features it shares in common with native cells in the absence of a genetic modification of cells. In some embodiments, the stem cell-derived cell is genetically modified.

In some aspects, the disclosure provides a cell line comprising a stem cell-derived cell described herein. In some aspects, the disclosure provides an SC-islet comprising stem cell-derived cells described herein (e.g., SC-β cells, SC-α cells, and/or SC-δ cells).

In some embodiments, the cells described herein, e.g. a population of stem cell-derived cells are transplantable, e.g., a population of stem cell-derived cells can be administered to a subject. In some embodiments, the subject who is administered a population of stem cell-derived cells is the same subject from whom a pluripotent stem cell used to differentiate into a stem cell-derived cell was obtained (e.g. for autologous cell therapy). In some embodiments, the subject is a different subject. In some embodiments, a subject is suffering from an intestinal disorder such as intestinal inflammation or is a normal subject. For example, the cells for transplantation (e.g. a composition comprising a population of stem cell-derived cells) can be a form suitable for transplantation, e.g., organ transplantation.

The method can further include administering the cells to a subject in need thereof, e.g., a mammalian subject, e.g., a human subject. The source of the cells can be a mammal, preferably a human. The source or recipient of the cells can also be a non-human subject, e.g., an animal model. The term “mammal” includes organisms, which include mice, rats, cows, sheep, pigs, rabbits, goats, horses, monkeys, dogs, cats, and preferably humans. Likewise, transplantable cells can be obtained from any of these organisms, including a non-human transgenic organism. In one embodiment, the transplantable cells are genetically engineered, e.g., the cells include an exogenous gene or have been genetically engineered to inactivate or alter an endogenous gene.

A composition comprising a population of stem cell-derived cells (e.g., pancreatic stem cell-derived cells, such as SC-β cells and/or SC-α cells) can be administered to a subject using an implantable device. Implantable devices and related technology are known in the art and are useful as delivery systems where a continuous, or timed-release delivery of compounds or compositions delineated herein is desired. Additionally, the implantable device delivery system is useful for targeting specific points of compound or composition delivery (e.g., localized sites, organs). Negrin et al., Biomaterials, 22(6):563 (2001). Timed-release technology involving alternate delivery methods can also be used in this invention. For example, timed-release formulations based on polymer technologies, sustained-release techniques and encapsulation techniques (e.g., polymeric, liposomal) can also be used for delivery of the compounds and compositions delineated herein.

For administration to a subject, a cell population produced by the methods as disclosed herein, e.g. a population of stem cell-derived cells can be administered to a subject, for example in pharmaceutically acceptable compositions. These pharmaceutically acceptable compositions comprise a therapeutically effective amount of a population of stem cell-derived cells as described above, formulated together with one or more pharmaceutically acceptable carriers (additives) and/or diluents.

As described in detail below, the pharmaceutical compositions of the present invention can be specially formulated for administration in solid or liquid form, including those adapted for the following: (1) oral administration, for example, drenches (aqueous or non-aqueous solutions or suspensions), lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; or (9) nasally. Additionally, compounds can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960.

As used here, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (24) C₂-C₁₂ alcohols, such as ethanol; and (25) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

The phrase “therapeutically-effective amount” as used herein in respect to a population of cells means that amount of relevant cells in a population of cells, e.g., stem cell-derived cells, or a composition comprising stem cell-derived cells of the present invention which is effective for producing some desired therapeutic effect in at least a sub-population of cells in an animal at a reasonable benefit/risk ratio applicable to any medical treatment. For example, an amount of a population of stem cell-derived cells administered to a subject that is sufficient to produce a statistically significant, measurable change in at least one symptom of Type 1, Type 1.5 or Type 2 diabetes, such as glycosylated hemoglobin level, fasting blood glucose level, hypoinsulinemia, etc. Determination of a therapeutically effective amount is well within the capability of those skilled in the art. Generally, a therapeutically effective amount can vary with the subject's history, age, condition, sex, as well as the severity and type of the medical condition in the subject, and administration of other pharmaceutically active agents.

As used herein, the term “administer” refers to the placement of a composition into a subject by a method or route which results in at least partial localization of the composition at a desired site such that desired effect is produced. A compound or composition described herein can be administered by any appropriate route known in the art including, but not limited to, oral or parenteral routes, including intravenous, intramuscular, subcutaneous, transdermal, airway (aerosol), pulmonary, nasal, rectal, and topical (including buccal and sublingual) administration.

Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In preferred embodiments, the compositions are administered by intravenous infusion or injection.

By “treatment,” “prevention,” or “amelioration” of a disease or disorder is meant delaying or preventing the onset of such a disease or disorder, reversing, alleviating, ameliorating, inhibiting, slowing down or stopping the progression, aggravation or deterioration of the progression or severity of a condition associated with such a disease or disorder. In one embodiment, the symptoms of a disease or disorder are alleviated by at least 5%, at least 10%, at least 20%, at least 30%, at least 40%, or at least 50%.

Treatment of diabetes is determined by standard medical methods. A goal of diabetes treatment is to bring sugar levels down to as close to normal as is safely possible. Commonly set goals are 80-120 milligrams per deciliter (mg/dl) before meals and 100-140 mg/dl at bedtime. A particular physician may set different targets for the patient, depending on other factors, such as how often the patient has low blood sugar reactions. Useful medical tests include tests on the patient's blood and urine to determine blood sugar level, tests for glycosylated hemoglobin level (HbA1c; a measure of average blood glucose levels over the past 2-3 months, normal range being 4-6%), tests for cholesterol and fat levels, and tests for urine protein level. Such tests are standard tests known to those of skill in the art (see, for example, American Diabetes Association, 1998). A successful treatment program can also be determined by having fewer patients in the program with complications relating to diabetes, such as diseases of the eye, kidney disease, or nerve disease.

Delaying the onset of diabetes in a subject refers to delay of onset of at least one symptom of diabetes, e.g., hyperglycemia, hypoinsulinemia, diabetic retinopathy, diabetic nephropathy, blindness, memory loss, renal failure, cardiovascular disease (including coronary artery disease, peripheral artery disease, cerebrovascular disease, atherosclerosis, and hypertension), neuropathy, autonomic dysfunction, hyperglycemic hyperosmolar coma, or combinations thereof, for at least 1 week, at least 2 weeks, at least 1 month, at least 2 months, at least 6 months, at least 1 year, at least 2 years, at least 5 years, at least 10 years, at least 20 years, at least 30 years, at least 40 years or more, and can include the entire lifespan of the subject.

In certain embodiments, the subject is a mammal, e.g., a primate, e.g., a human. The terms, “patient” and “subject” are used interchangeably herein. Preferably, the subject is a mammal. The mammal can be a human, non-human primate, mouse, rat, dog, cat, horse, or cow, but are not limited to these examples. Mammals other than humans can be advantageously used as subjects that represent animal models of Type 1 diabetes, Type 2 Diabetes Mellitus, or pre-diabetic conditions. In addition, the methods described herein can be used to treat domesticated animals and/or pets. A subject can be male or female. A subject can be one who has been previously diagnosed with or identified as suffering from or having diabetes (e.g., Type 1 or Type 2), one or more complications related to diabetes, or a pre-diabetic condition, and optionally, but need not have already undergone treatment for diabetes, the one or more complications related to diabetes, or the pre-diabetic condition. A subject can also be one who is not suffering from diabetes or a pre-diabetic condition. A subject can also be one who has been diagnosed with or identified as suffering from diabetes, one or more complications related to diabetes, or a pre-diabetic condition, but who show improvements in known diabetes risk factors as a result of receiving one or more treatments for diabetes, one or more complications related to diabetes, or the pre-diabetic condition. Alternatively, a subject can also be one who has not been previously diagnosed as having diabetes, one or more complications related to diabetes, or a pre-diabetic condition. For example, a subject can be one who exhibits one or more risk factors for diabetes, complications related to diabetes, or a pre-diabetic condition, or a subject who does not exhibit diabetes risk factors, or a subject who is asymptomatic for diabetes, one or more diabetes-related complications, or a pre-diabetic condition. A subject can also be one who is suffering from or at risk of developing diabetes or a pre-diabetic condition. A subject can also be one who has been diagnosed with or identified as having one or more complications related to diabetes or a pre-diabetic condition as defined herein, or alternatively, a subject can be one who has not been previously diagnosed with or identified as having one or more complications related to diabetes or a pre-diabetic condition.

As used herein, the phrase “subject in need of pancreatic stem cell-derived cells” refers to a subject who is diagnosed with or identified as suffering from, having or at risk for developing diabetes (e.g., Type 1, Type 1.5 or Type 2), one or more complications related to diabetes, or a pre-diabetic condition.

A subject in need of a population of pancreatic stem cell-derived cells can be identified using any method used for diagnosis of diabetes. For example, Type 1 diabetes can be diagnosed using a glycosylated hemoglobin (A1C) test, a random blood glucose test and/or a fasting blood glucose test. Parameters for diagnosis of diabetes are known in the art and available to skilled artisan without much effort.

In some embodiments, the methods of the invention further comprise selecting a subject identified as being in need of additional pancreatic stem cell-derived cells. A subject in need a population of pancreatic stem cell-derived cells can be selected based on the symptoms presented, such as symptoms of type 1, type 1.5 or type 2 diabetes. Exemplary symptoms of diabetes include, but are not limited to, excessive thirst (polydipsia), frequent urination (polyuria), extreme hunger (polyphagia), extreme fatigue, weight loss, hyperglycemia, low levels of insulin, high blood sugar (e.g., sugar levels over 250 mg, over 300 mg), presence of ketones present in urine, fatigue, dry and/or itchy skin, blurred vision, slow healing cuts or sores, more infections than usual, numbness and tingling in feet, diabetic retinopathy, diabetic nephropathy, blindness, memory loss, renal failure, cardiovascular disease (including coronary artery disease, peripheral artery disease, cerebrovascular disease, atherosclerosis, and hypertension), neuropathy, autonomic dysfunction, hyperglycemic hyperosmolar coma, and combinations thereof.

In some embodiments, a composition comprising a population of stem cell-derived cells for administration to a subject can further comprise a pharmaceutically active agent, such as those agents known in the art for treatment of diabetes and or for having anti-hyperglycemic activities, for example, inhibitors of dipeptidyl peptidase 4 (DPP-4) (e.g., Alogliptin, Linagliptin, Saxagliptin, Sitagliptin, Vildagliptin, and Berberine), biguanides (e.g., Metformin, Buformin and Phenformin), peroxisome proliferator-activated receptor (PPAR) modulators such as thiazolidinediones (TZDs) (e.g., Pioglitazone, Rivoglitazone, Rosiglitazone and Troglitazone), dual PPAR agonists (e.g., Aleglitazar, Muraglitazar and Tesaglitazar), sulfonylureas (e.g., Acetohexamide, Carbutamide, Chlorpropamide, Gliclazide, Tolbutamide, Tolazamide, Glibenclamide (Glyburide), Glipizide, Gliquidone, Glyclopyramide, and Glimepiride), meglitinides (“glinides”) (e.g., Nateglinide, Repaglinide and Mitiglinide), glucagon-like peptide-1 (GLP-1) and analogs (e.g., Exendin-4, Exenatide, Liraglutide, Albiglutide), insulin and insulin analogs (e.g., Insulin lispro, Insulin aspart, Insluin glulisine, Insulin glargine, Insulin detemir, Exubera and NPH insulin), alpha-glucosidase inhibitors (e.g., Acarbose, Miglitol and Voglibose), amylin analogs (e.g. Pramlintide), Sodium-dependent glucose cotransporter T2 (SGLT T2) inhibitors (e.g., Dapgliflozin, Remogliflozin and Sergliflozin) and others (e.g. Benfluorex and Tolrestat).

A composition comprising stem cell-derived cells can be administrated to the subject at the same time or at different times as the administration of a pharmaceutically active agent or composition comprising the same. When administrated at different times, the compositions comprising a population of stem cell-derived cells and/or pharmaceutically active agent for administration to a subject can be administered within 5 minutes, 10 minutes, 20 minutes, 60 minutes, 2 hours, 3 hours, 4, hours, 8 hours, 12 hours, 24 hours of administration of the other. When a composition comprising a population of stem cell-derived cells and a composition comprising a pharmaceutically active agent are administered in different pharmaceutical compositions, routes of administration can be different. In some embodiments, a subject is administered a composition comprising stem cell-derived cells. In other embodiments, a subject is administered a composition comprising a pharmaceutically active agent. In another embodiment, a subject is administered a composition comprising a population of stem cell-derived cells mixed with a pharmaceutically active agent. In another embodiment, a subject is administered a composition comprising a population of stem cell-derived cells and a composition comprising a pharmaceutically active agent, where administration is substantially at the same time, or subsequent to each other.

Toxicity and therapeutic efficacy of administration of a composition comprising a population of stem cell-derived cells can be determined by standard pharmaceutical procedures in cell cultures or experimental animals, e.g., for determining the LD50 (the dose lethal to 50% of the population) and the ED50 (the dose therapeutically effective in 50% of the population). Compositions comprising a population of stem cell-derived cells that exhibit large therapeutic indices are preferred.

The amount of a composition comprising a population of stem cell-derived cells can be tested using several well-established animal models.

The non-obese diabetic (NOD) mouse carries a genetic defect that results in insulitis showing at several weeks of age (Yoshida et al., Rev. Immunogenet. 2:140, 2000). 60-90% of the females develop overt diabetes by 20-30 weeks. The immune-related pathology appears to be similar to that in human Type I diabetes. Other models of Type I diabetes are mice with transgene and knockout mutations (Wong et al., Immunol. Rev. 169:93, 1999). A rat model for spontaneous Type I diabetes was recently reported by Lenzen et al. (Diabetologia 44:1189, 2001). Hyperglycemia can also be induced in mice (>500 mg glucose/dL) by way of a single intraperitoneal injection of streptozotocin (Soria et al., Diabetes 49:157, 2000), or by sequential low doses of streptozotocin (Ito et al., Environ. Toxicol. Pharmacol. 9:71, 2001). To test the efficacy of implanted islet cells, the mice are monitored for return of glucose to normal levels (<200 mg/dL).

Larger animals provide a good model for following the sequelae of chronic hyperglycemia. Dogs can be rendered insulin-dependent by removing the pancreas (J. Endocrinol. 158:49, 2001), or by feeding galactose (Kador et al., Arch. Opthalmol. 113:352, 1995). There is also an inherited model for Type I diabetes in keeshond dogs (Am. J. Pathol. 105:194, 1981). Early work with a dog model (Banting et al., Can. Med. Assoc. J. 22:141, 1922) resulted in a couple of Canadians making a long ocean journey to Stockholm in February of 1925.

In some embodiments, data obtained from the cell culture assays and in animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized.

The therapeutically effective dose of a composition comprising a population of stem cell-derived cells can also be estimated initially from cell culture assays. Alternatively, the effects of any particular dosage can be monitored by a suitable bioassay.

With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment or make other alteration to treatment regimen. The dosing schedule can vary from once a week to daily depending on a number of clinical factors, such as the subject's sensitivity to the stem cell-derived cells. The desired dose can be administered at one time or divided into subdoses, e.g., 2-4 subdoses and administered over a period of time, e.g., at appropriate intervals through the day or other appropriate schedule. Such sub-doses can be administered as unit dosage forms. In some embodiments, administration is chronic, e.g., one or more doses daily over a period of weeks or months. Examples of dosing schedules are administration daily, twice daily, three times daily or four or more times daily over a period of 1 week, 2 weeks, 3 weeks, 4 weeks, 1 month, 2 months, 3 months, 4 months, 5 months, or 6 months or more.

In another aspect of the invention, the methods provide use of an isolated population of stem cell-derived cells as disclosed herein. In one embodiment of the invention, an isolated population of stem cell-derived cells as disclosed herein may be used for the production of a pharmaceutical composition, for use in transplantation into subjects in need of treatment, e.g. a subject that has, or is at risk of developing diabetes, for example but not limited to subjects with congenital and acquired diabetes. In one embodiment, an isolated population of stem cell-derived cells may be genetically modified. In another aspect, the subject may have or be at risk of diabetes and/or metabolic disorder. In some embodiments, an isolated population of stem cell-derived cells as disclosed herein may be autologous and/or allogeneic. In some embodiments, the subject is a mammal, and in other embodiments the mammal is a human.

The use of an isolated population of stem cell derived cells as disclosed herein provides advantages over existing methods because the population of stem cell-derived cells can be differentiated from endocrine progenitor cells or precursors thereof derived from stem cells, e.g. iPS cells obtained or harvested from the subject administered an isolated population of stem cell-derived cells. This is highly advantageous as it provides a renewable source of stem cell-derived cells which can be differentiated from stem cells to endocrine progenitor cells by methods commonly known by one of ordinary skill in the art, and then further differentiated by the methods described herein to pancreatic a-like cells, pancreatic j-like cells, or cells with pancreatic a or 3 cell characteristics, for transplantation into a subject, in particular a substantially pure population of mature pancreatic a-like cells or pancreatic j-like cells that do not have the risks and limitations of cells derived from other systems.

One embodiment of the invention relates to a method of treating diabetes or a metabolic disorder in a subject comprising administering an effective amount of a composition comprising a population of stem cell-derived cells (e.g., pancreatic stem cell-derived cells) as disclosed herein to a subject with diabetes and/or a metabolic disorder. In a further embodiment, the invention provides a method for treating diabetes, comprising administering a composition comprising a population of stem cell-derived cells as disclosed herein to a subject that has, or has an increased risk of developing diabetes.

In one embodiment of the above methods, the subject is a human and a population of stem cell-derived cells as disclosed herein are human cells. In some embodiments, the invention contemplates that a population of stem cell-derived cells as disclosed herein are administered directly to the pancreas of a subject or is administered systemically. In some embodiments, a population of stem cell-derived cells as disclosed herein can be administered to any suitable location in the subject, for example in a capsule in the blood vessel or the liver.

The present invention is also directed to a method of treating a subject with diabetes or a metabolic disorder which occurs as a consequence of genetic defect, physical injury, environmental insult or conditioning, bad health, obesity and other diabetes risk factors commonly known by a person of ordinary skill in the art. Efficacy of treatment of a subject administered a composition comprising a population of stem cell-derived cells (e.g., pancreatic stem cell-derived cells) can be monitored by clinically accepted criteria and tests, which include for example, (i) Glycated hemoglobin (A IC) test, which indicates a subjects average blood sugar level for the past two to three months, by measuring the percentage of blood sugar attached to hemoglobin, the oxygen-carrying protein in red blood cells. The higher your blood sugar levels, the more hemoglobin has sugar attached. An A1C level of 6.5 percent or higher on two separate tests indicates the subject has diabetes. A test value of 6-6.5% suggest the subject has prediabetes. (ii) Random blood sugar test. A blood sample will be taken from the subject at a random time, and a random blood sugar level of 200 milligrams per deciliter (mg/dL)-11.1 millimoles per liter (mmol/L), or higher indicated the subject has diabetes. (iii) Fasting blood sugar test. A blood sample is taken from the subject after an overnight fast. A fasting blood sugar level between 70 and 99 mg/dL (3.9 and 5.5 mmol/L) is normal. If the subjects fasting blood sugar levels is 126 mg/dL (7 mmol/L) or higher on two separate tests, the subject has diabetes. A blood sugar level from 100 to 125 mg/dL (5.6 to 6.9 mmol/L) indicates the subject has prediabetes. (iv) Oral glucose tolerance test. A blood sample will be taken after the subject has fasted for at least eight hours or overnight and then ingested a sugary solution, and the blood sugar level will be measured two hours later. A blood sugar level less than 140 mg/dL (7.8 mmol/L) is normal. A blood sugar level from 140 to 199 mg/dL (7.8 to 11 mmol/L) is considered prediabetes. This is sometimes referred to as impaired glucose tolerance (IGT). A blood sugar level of 200 mg/dL (11.1 mmol/L) or higher may indicate diabetes.

In some embodiments, the effects of administration of a population of stem cell-derived cells (e.g., pancreatic stem cell-derived cells) as disclosed herein to a subject in need thereof is associated with improved exercise tolerance or other quality of life measures, and decreased mortality. The effects of cellular therapy with a population of stem cell-derived cells can be evident over the course of days to weeks after the procedure. However, beneficial effects may be observed as early as several hours after the procedure and may persist for several years. In some embodiments, the effects of cellular therapy with a population of stem cell-derived cells occurs within two weeks after the procedure.

In some embodiments, a population of stem cell-derived cells (e.g., pancreatic stem cell-derived cells) as disclosed herein may be used for tissue reconstitution or regeneration in a human patient or other subject in need of such treatment. In some embodiments compositions of populations of stem cell-derived cells can be administered in a manner that permits them to graft or migrate to the intended tissue site and reconstitute or regenerate the functionally deficient area. Special devices are available that are adapted for administering cells capable of reconstituting a population of pancreatic cells (e.g., α cells and/or β cells) in the pancreas or at an alternative desired location. Accordingly, the stem cell-derived cells may be administered to a recipient subject's pancreas by injection or administered by intramuscular injection.

### Example

### Example 1: Charting Cellular Identity During Human In Vitro Beta Cell Differentiation

In vitro differentiation of human stem cells can produce pancreatic beta cells, the insulin-secreting cell type whose loss underlies Type 1 Diabetes. As a step toward mastery of this process, a report on transcriptional profiling of >100,000 individual cells sampled during in vitro beta cell differentiation is provided and describes the cells that emerge. Populations are resolved corresponding to beta cells, alpha-like poly-hormonal cells, non-endocrine cells that resemble pancreatic exocrine cells and a previously unreported population resembling enterochromaffin cells. It is shown that endocrine cells maintain their identity in culture without exogenous growth factors and that gene expression changes associated with in vivo beta cell maturation are recapitulated in vitro. A scalable re-aggregation technique is implemented to deplete non-endocrine cells and identify CD49a/ITGA1 as a surface marker for the beta population allowing magnetic sorting to a purity of 80%. Finally, a high-resolution sequencing time course is utilized to characterize gene expression dynamics during human pancreatic endocrine induction from which a lineage model of in vitro beta cell differentiation is developed. This study provides a deeper perspective on the current state of human stem cell differentiation and will guide future endeavors on differentiation of pancreatic islet cells and their application in regenerative medicine.

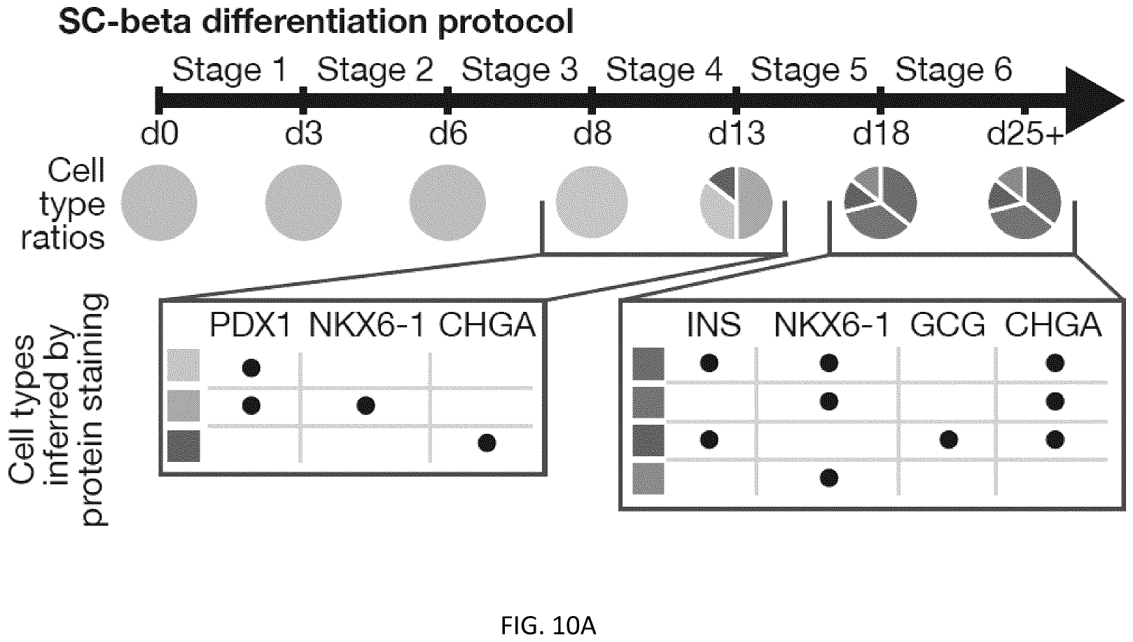

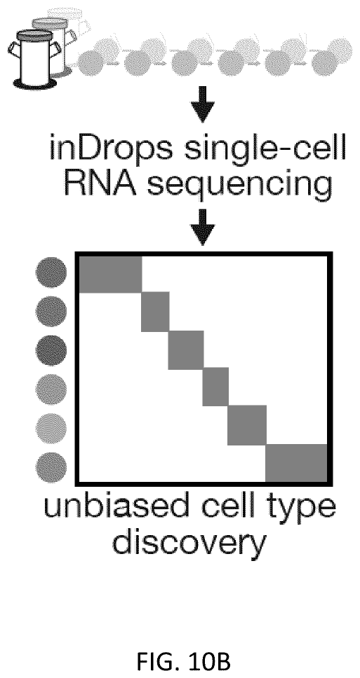

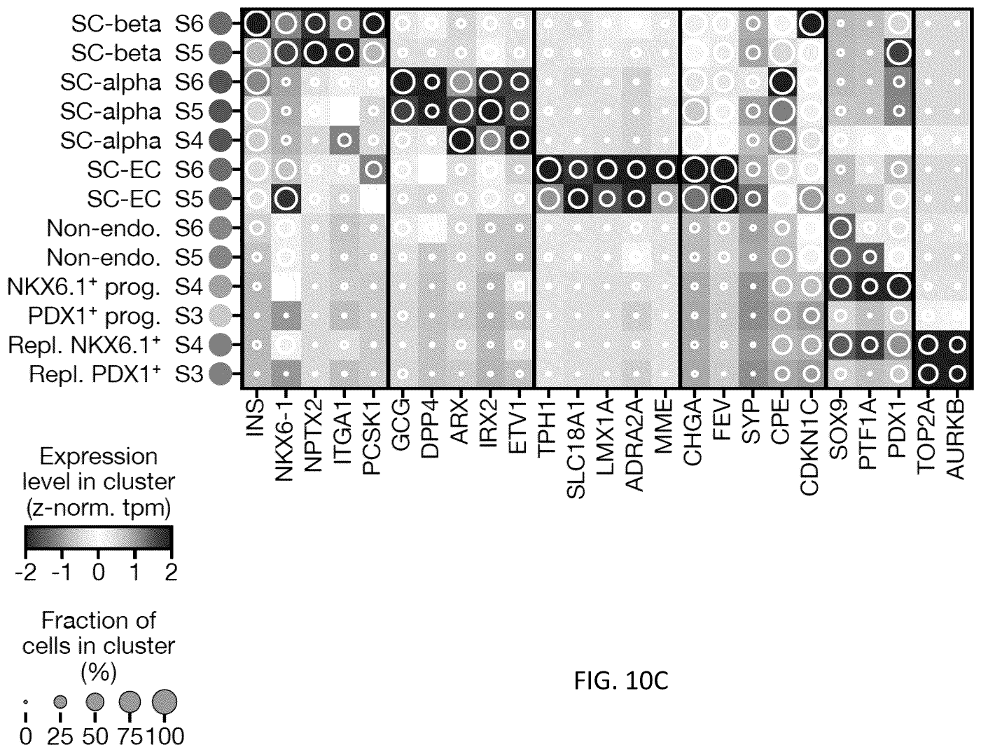

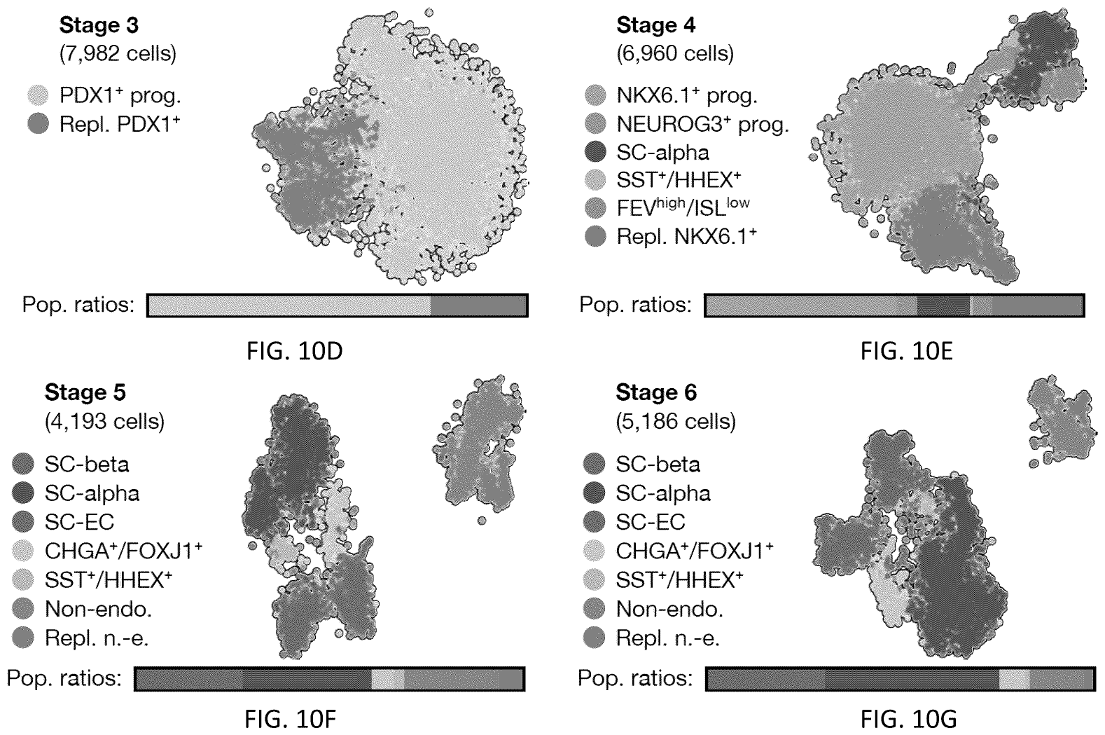

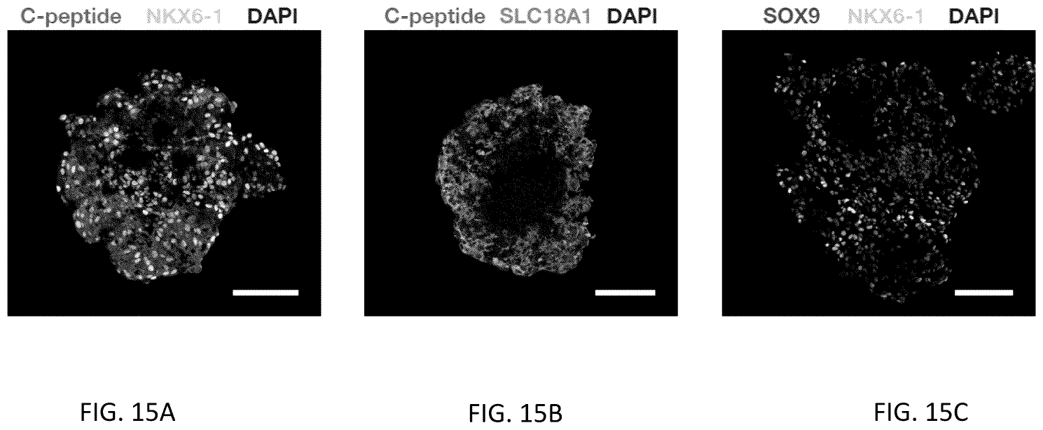

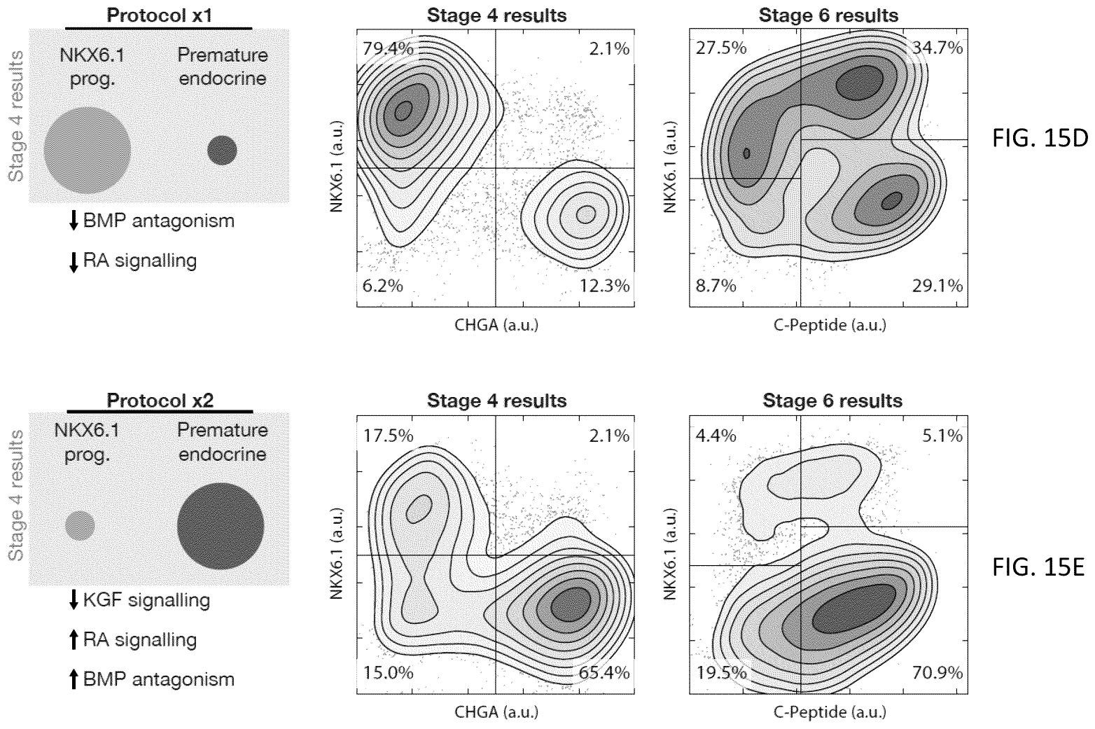

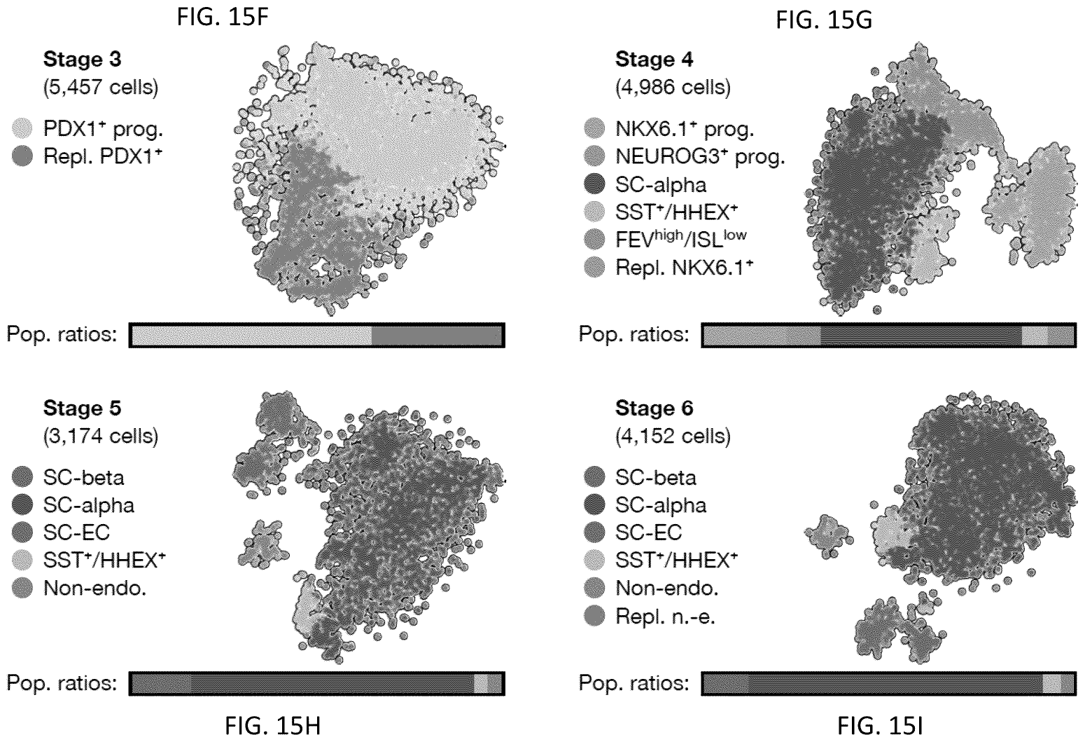

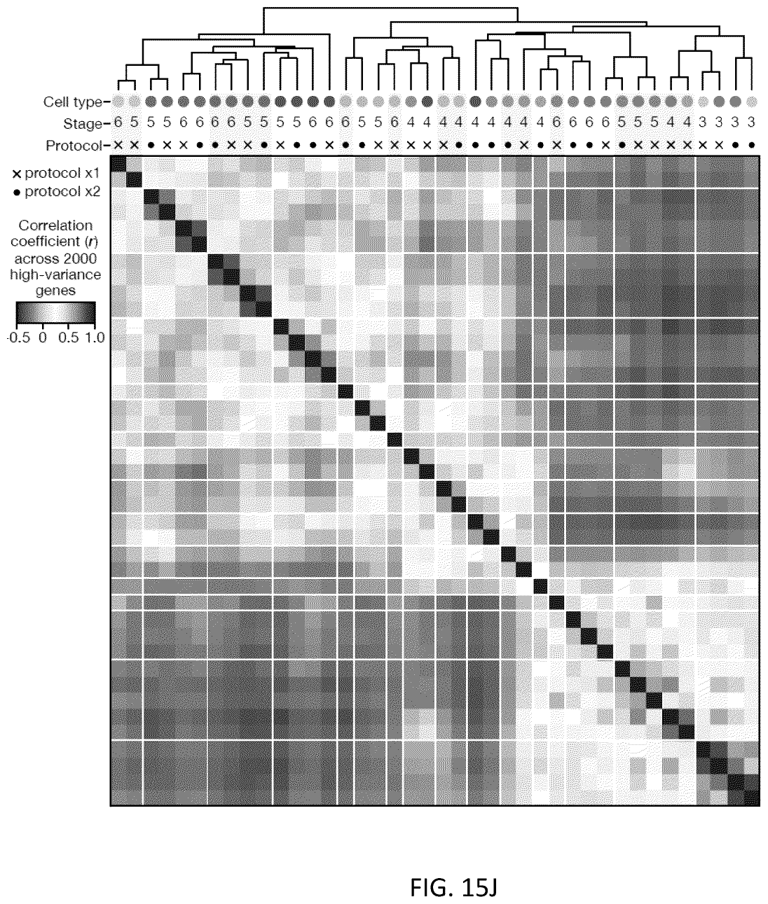

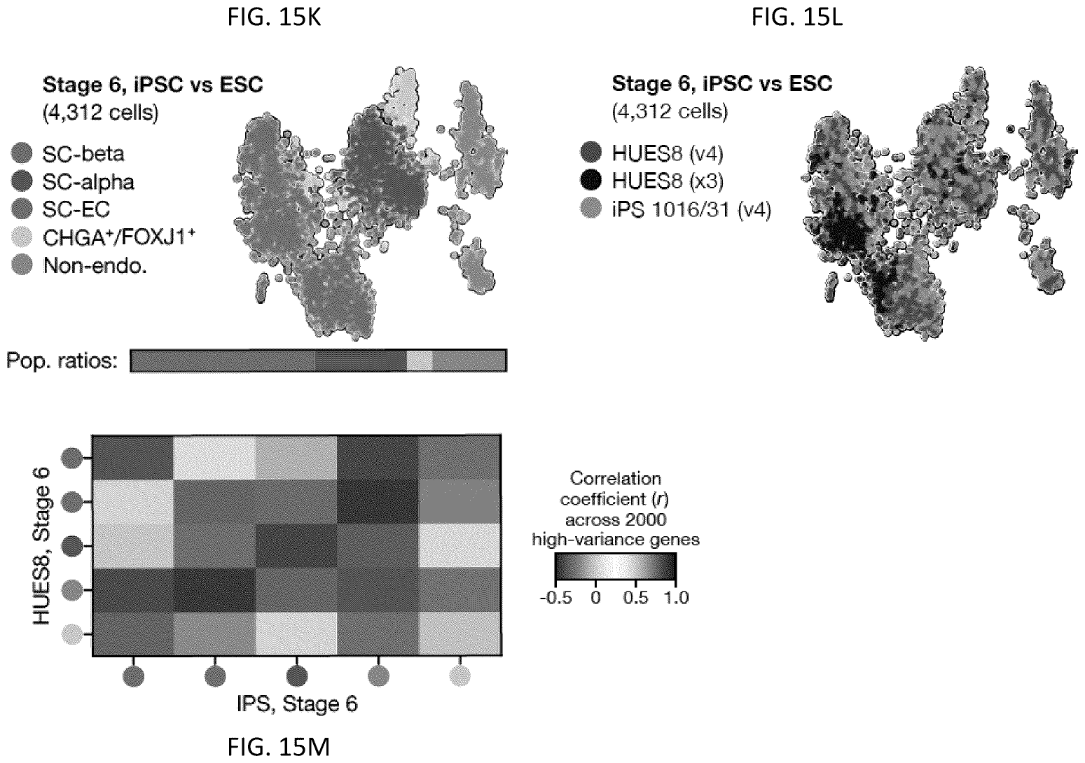

In the SC-beta protocol, human pluripotent stem cells grown in 3D clusters are differentiated with 6 stages with specific inducing factors to produce ‘SC-islets’ that contain stem cell-derived beta cells. Progress and efficiency are measured using immunofluorescence microscopy and flow cytometry (FIG. 10A). The first three stages of differentiation generate a nearly homogenous (˜90%) population of progenitors expressing the master transcription factor PDX1. Thereafter, distinct populations are identified by staining for C-peptide (a fragment of proinsulin), the pan-endocrine marker CHGA, and the beta cell transcription factor NKX6.1 (FIG. 10A, FIG. 15A).

Here a single cell RNA sequencing and computational analysis is applied to generate a deep understanding of in vitro beta cell differentiation (FIG. 10B). Emergent cell types are defined at each stage of differentiation through their global gene expression profiles, creating a precise, cell-by-cell description of in vitro beta cell differentiation. These are critical steps in advancing directed differentiation of stem cells toward a treatment for diabetes.

### SC-Islets Contain 4 Major Cell Types

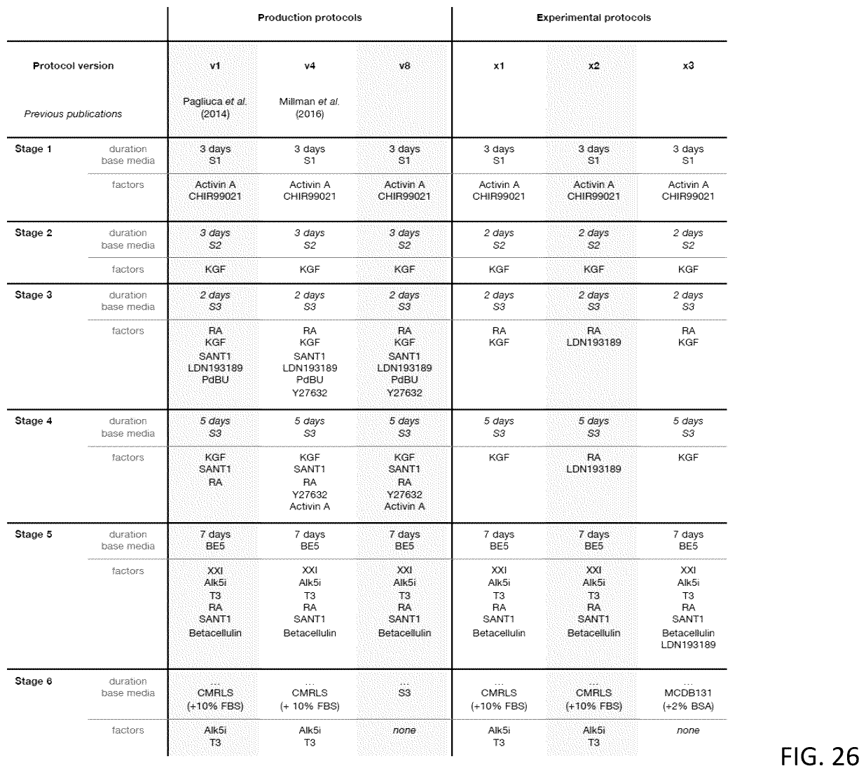

40,444 cells sampled from the ends of Stages 3 through 6 from differentiations done with two modified protocols were sequenced to define cell populations using their entire transcriptomes. These two protocols use subsets of the original¹ v1 Stages 3 and 4 factors and yield different populations ratios at Stage 4 (FIGS. 15D-15E, FIG. 26). Throughout this study, the fact that SC-beta differentiation is carried out in 3D suspension culture was leveraged to repeatedly sample the same differentiation over time.

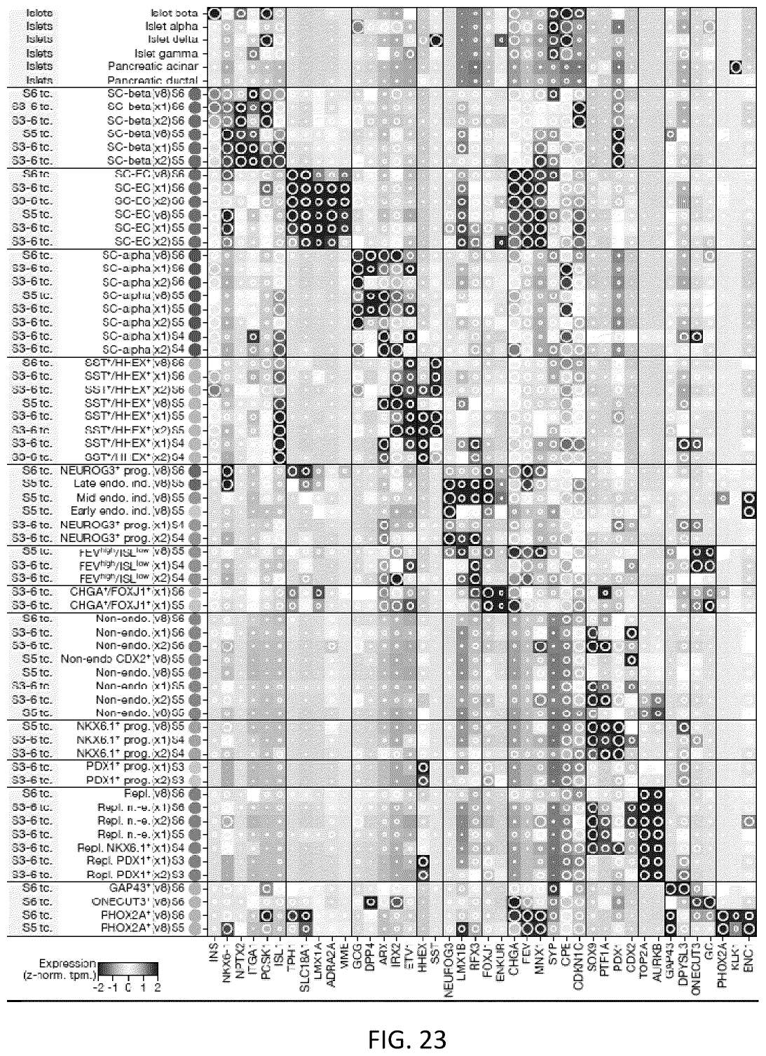

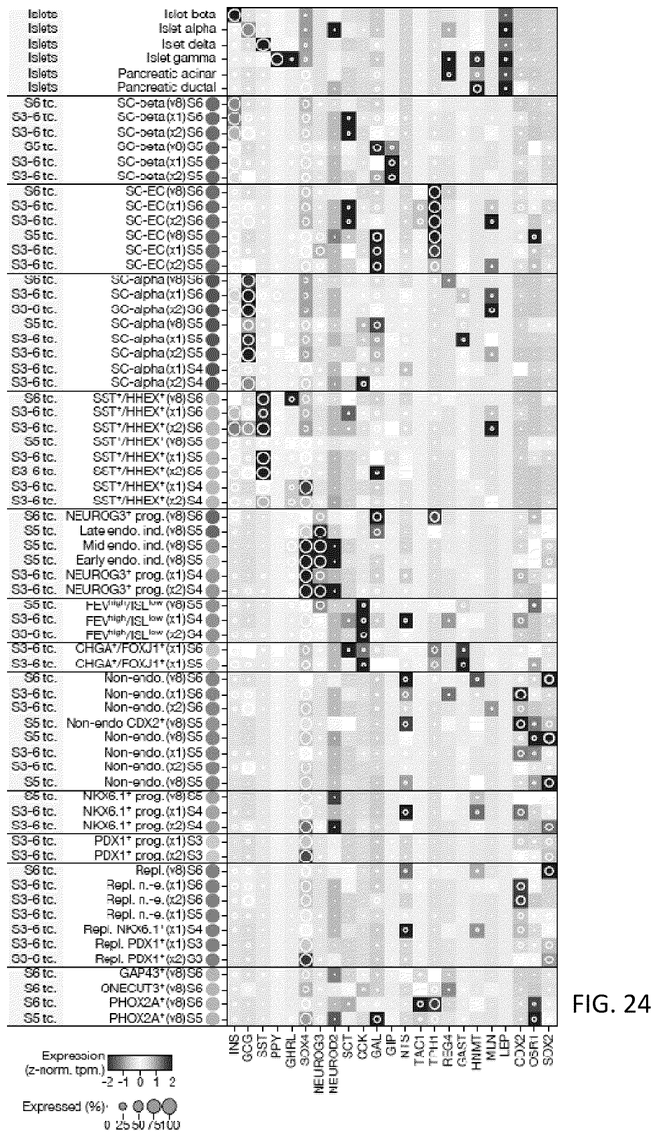

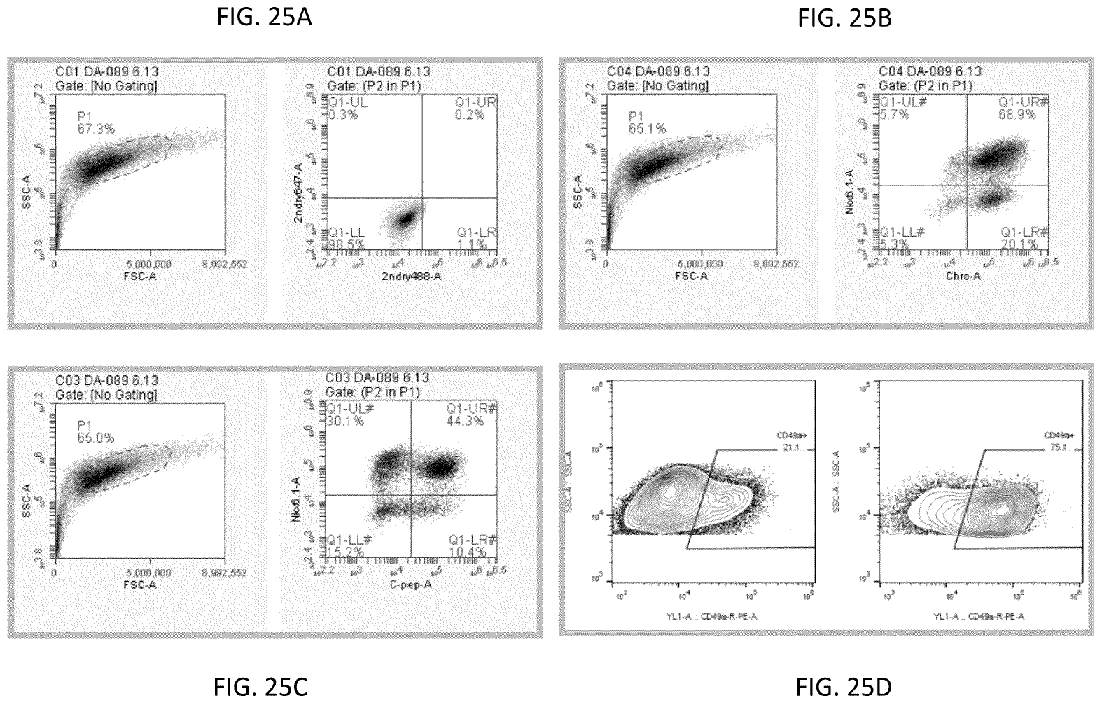

The major populations identified (FIGS. 10C-10G, FIG. 23) are progenitors (in Stages 3 & 4), three types of endocrine cells (Stages 4, 5 & 6) and one type of non-endocrine cell (Stages 5 & 6). In both protocols, cells at Stage 3 comprise a single population of replicating pancreatic progenitors (PDX1V). By the end of Stage 4, NKX6.1⁺ progenitors are observed as well as the first alpha-like cells. Finally, at Stages 5 and 6, three classes of CHGA+ endocrine cells are observed: (i)SC-beta cells, expressing INS, NKX6.1, ISL1 and other beta cell markers, (ii) alpha-like cells expressing GCG, ARX, IRX2 but also INS, and (iii) an endocrine cell type expressing CHGA, TPH1, LMX1A, SLC18A1 that most resembles enterochromaffin cells (SC-EC, FIG. 15B). At Stages 5 and 6, SOX9+ non-endocrine cells (FIG. 15C) form a final population with significant heterogeneity. Thus, two cell populations were identified with translational relevance corresponding to adult islet cell types (SC-beta and SC-alpha cells), alongside two other populations (SC-EC and non-endocrine cells).

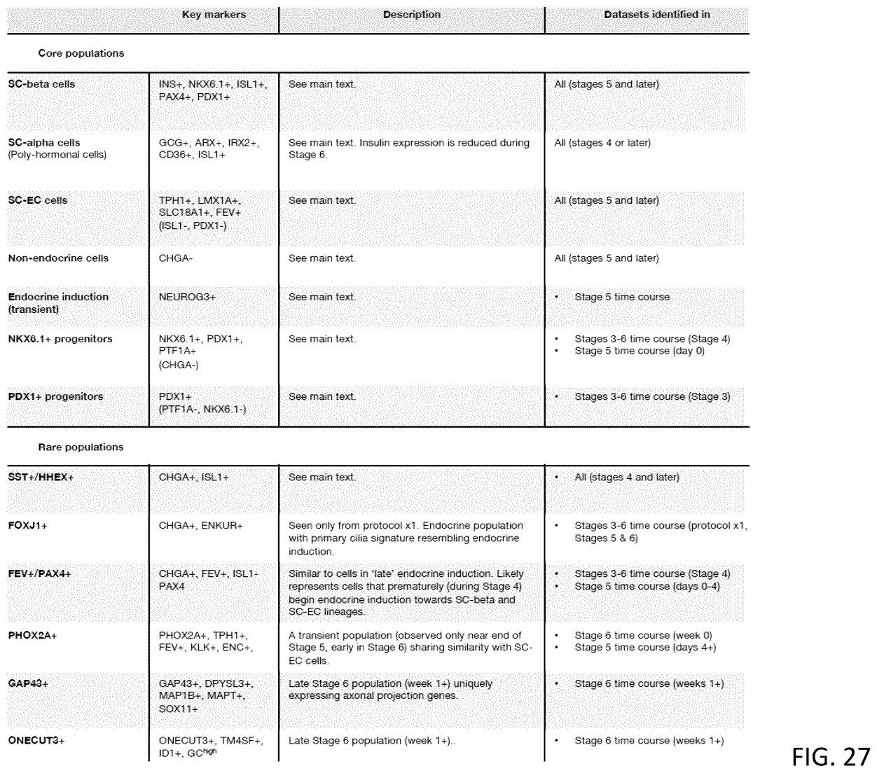

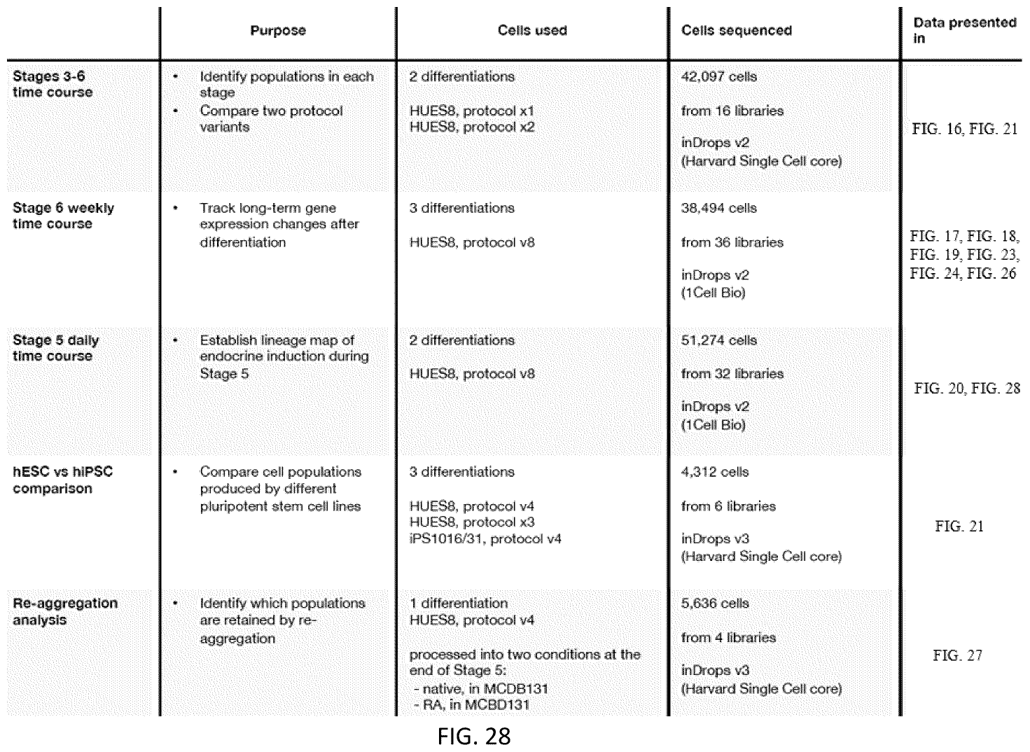

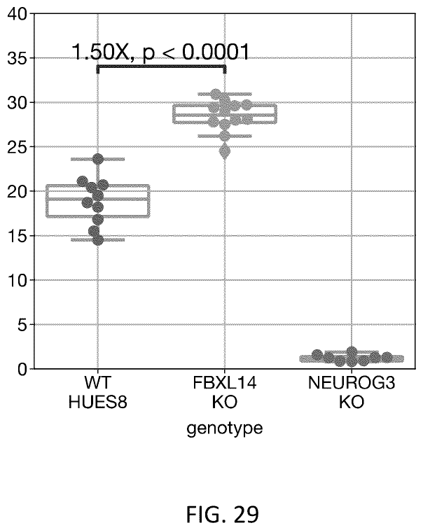

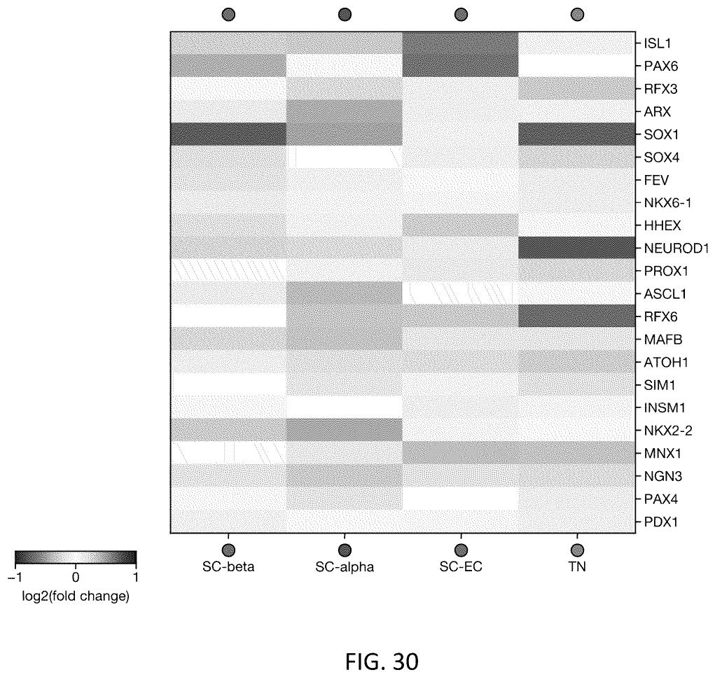

Beyond these major populations, both protocols include a small population of SST+/HHEX+/ISL1+ cells that emerge as early as the end of Stage 4. A single population, labelled by high levels of FOXJ1+, was present in only one protocol (FIG. 27). Although the protocol variants showed the expected large differences in cell type ratios (FIGS. 10D-10G, FIGS. 15F-15I), every cell type that was shared across protocols showed a similar gene expression signature (FIG. 15J). It is concluded that population ratios can be significantly affected by protocol modifications without altering the cell types' identities.

Finally, Stage 6 cells produced from differentiation of embryonic stem cells (ESCs, line HUES8) were compared to induced pluripotent stem cells (iPSCs, line 1016/31) and high correlations were observed between the corresponding cell types (FIGS. 15K-15M). Together, these results establish that the in vitro beta cell differentiation protocols guide a lineage progression that is robust to perturbation in differentiation factors and stem cell lines.

### SC-Beta Cells Stably Maintain Identity

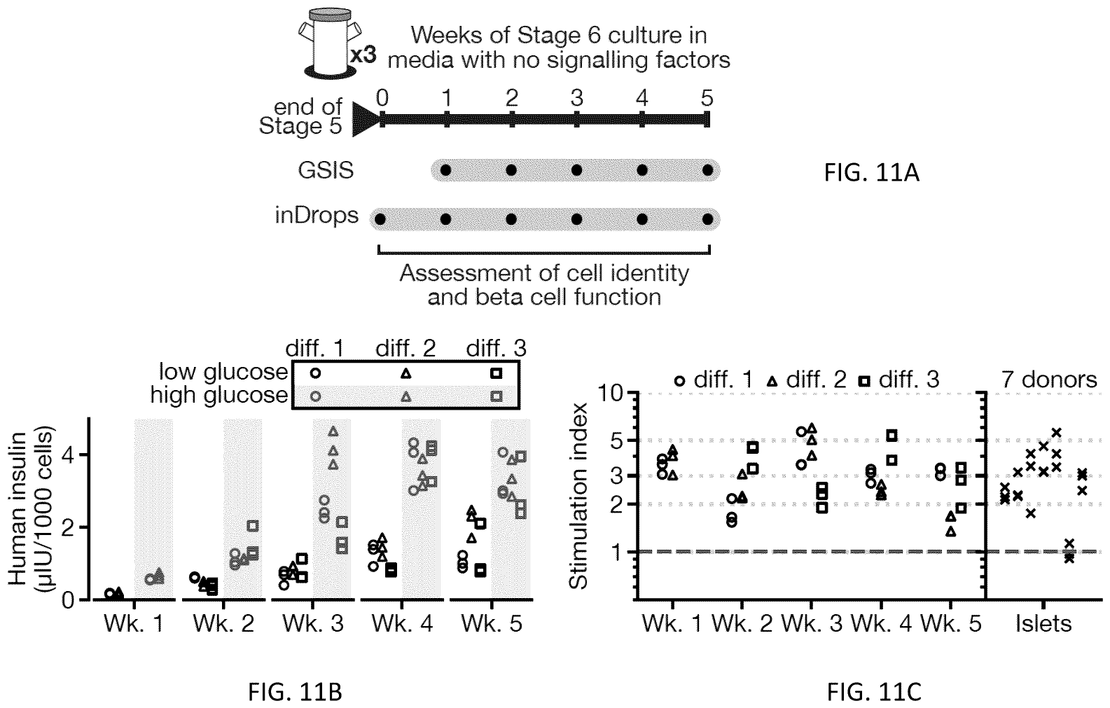

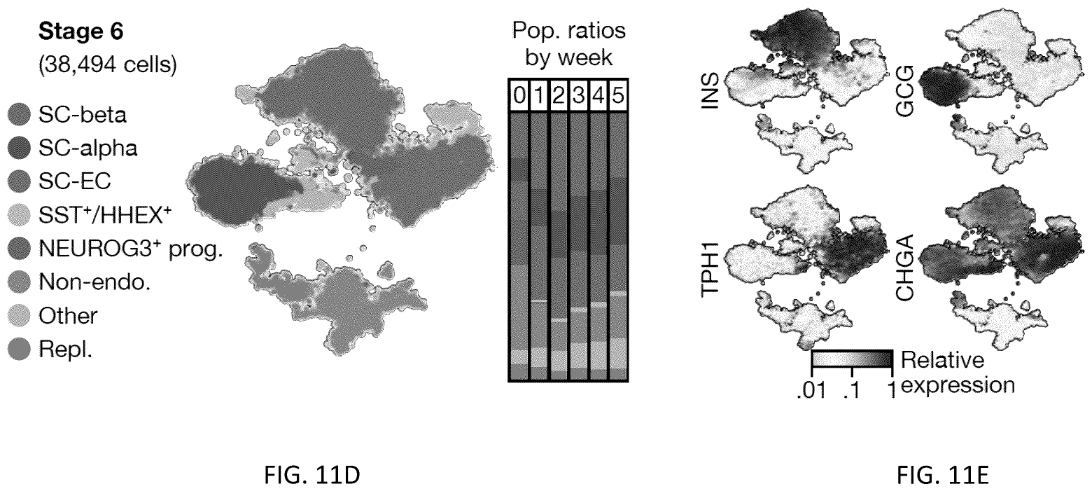

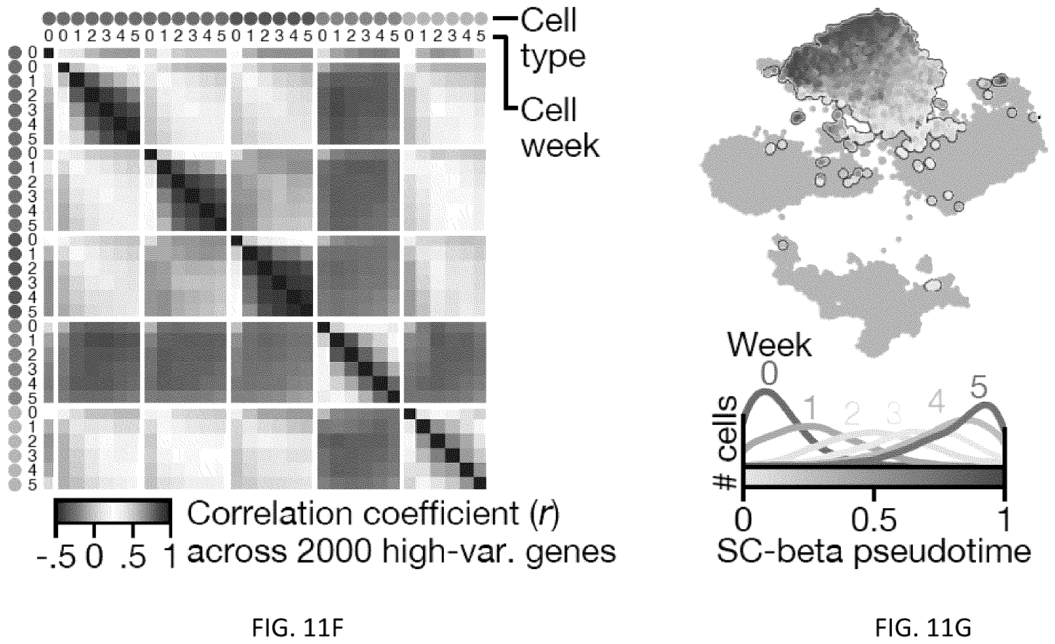

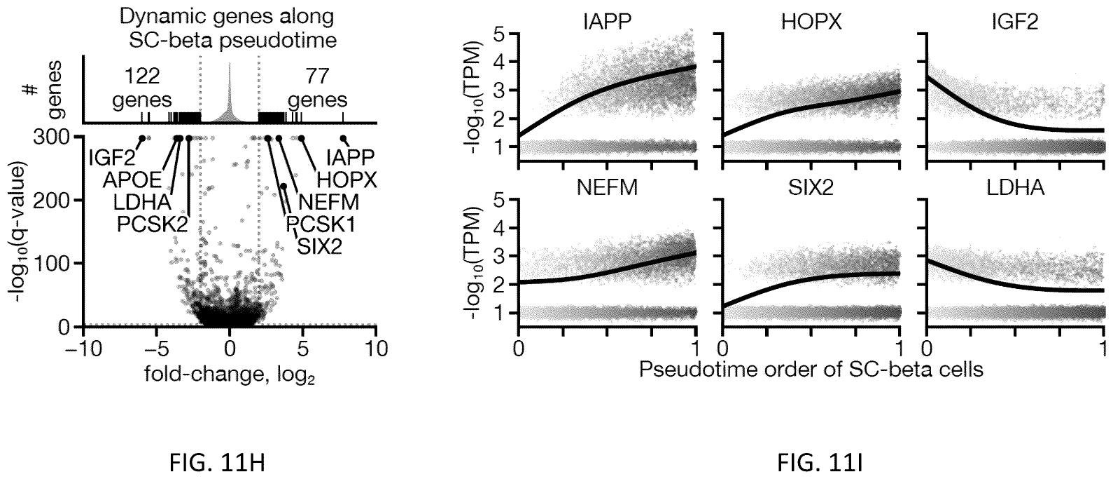

The key properties of SC-beta cells are glucose responsiveness and transcriptional similarity to endogenous human beta cells. These properties were characterized across several weeks of Stage 6, using serum-free media without exogenous signaling factors (protocol v8). Single cell RNA sequencing and in vitro glucose stimulated insulin secretion (GSIS) tests were carried out across several weeks of Stage 6, sampling at weekly intervals from three differentiations (FIG. 11A).

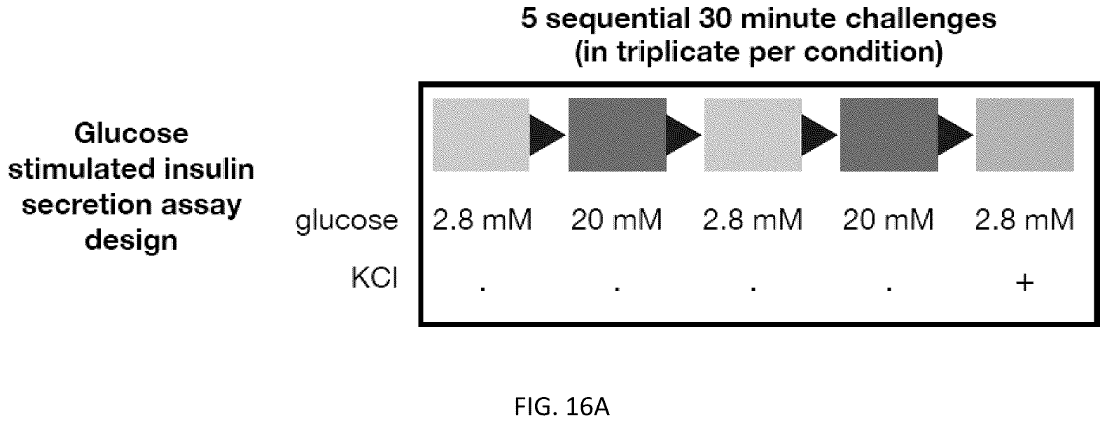

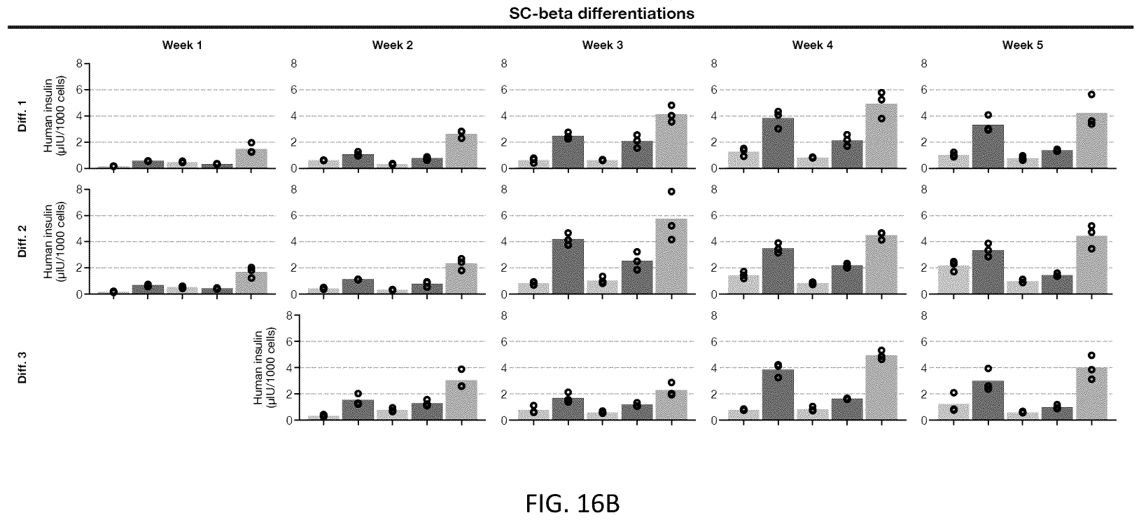

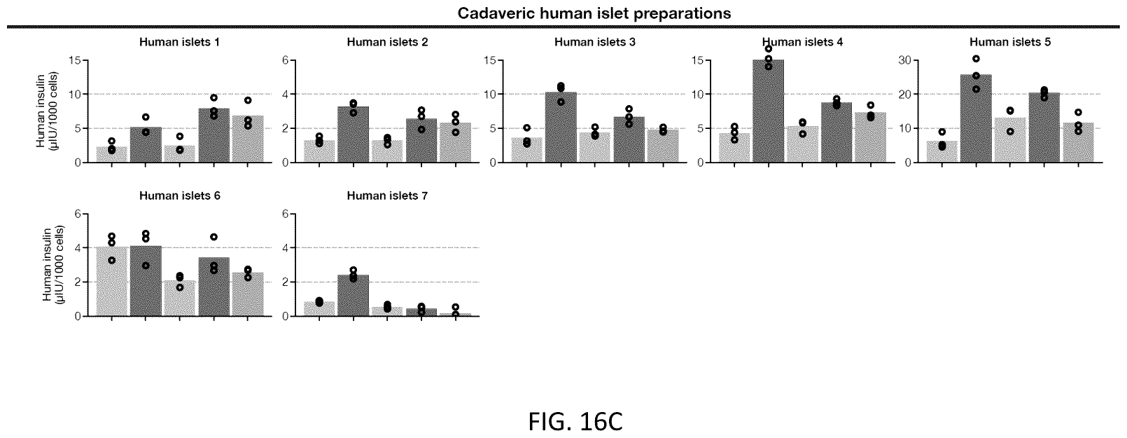

SC-islets acquire glucose responsive insulin secretion in the first week of Stage 6 and retain this ability for another ˜4 weeks (FIGS. 11B-11C, FIG. 16). The observed stimulation indices were in the same range as human islet controls, although the magnitude of secretion was higher in islets. These results show that glucose responsiveness is a stable trait, requiring no exogenous factors or serum.

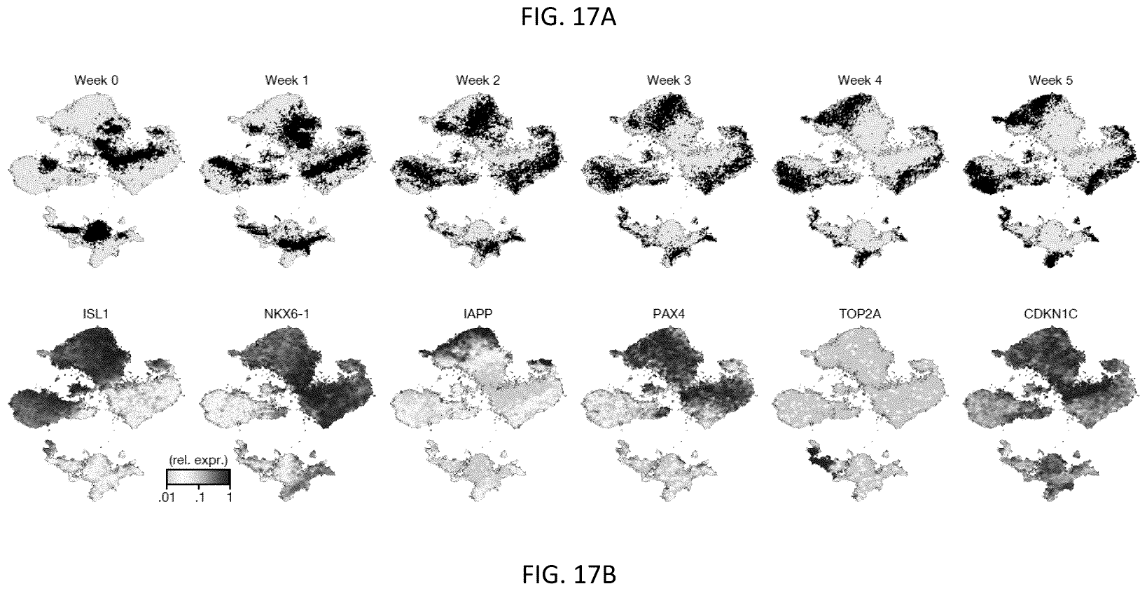

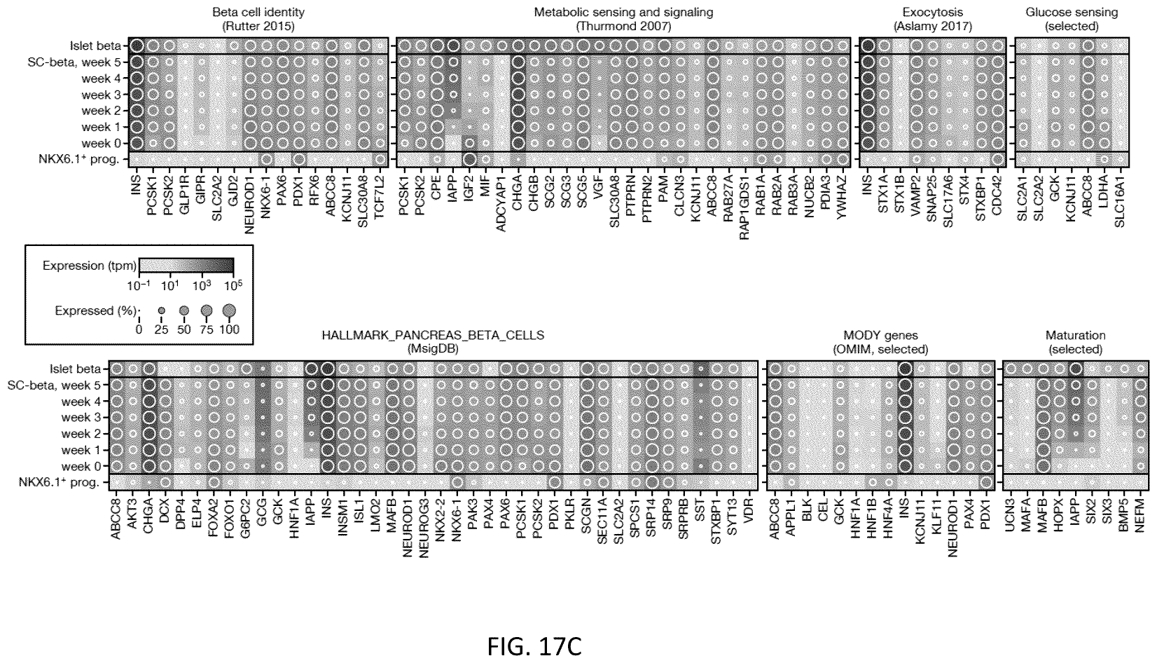

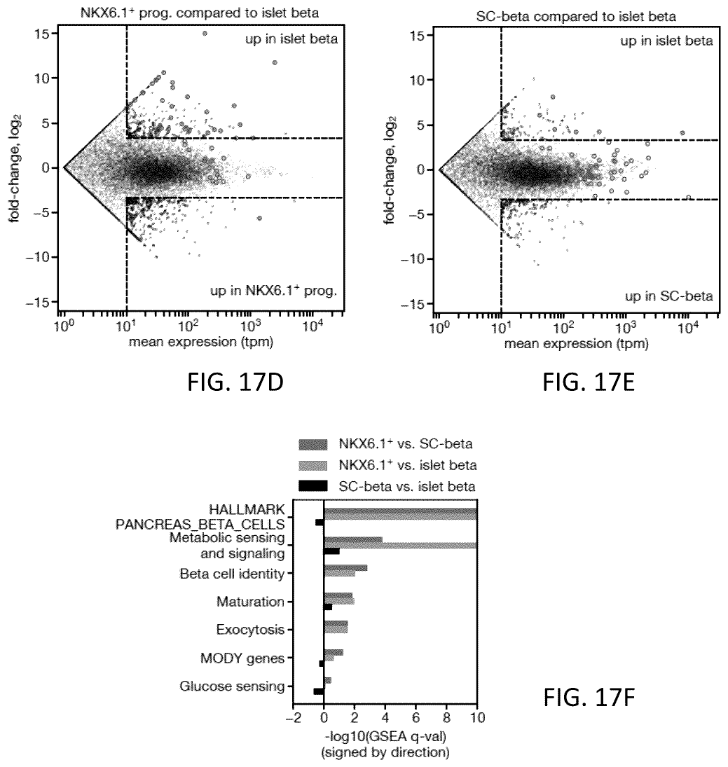

In parallel, whether the Stage 6 cell populations maintain their identity during extended time in culture was assessed. As in the previous dataset, SC-beta, SC-alpha, SC-EC cells and non-endocrine cells are identified (FIGS. 11D-11E, FIGS. 17A-17B). Small, rare populations (FIG. 27) are present only at week 0 and then disappear (PHOX2A+), or are first detected late in Stage 6 (GAP43+, ONECUT3+). SST+/HHEX+ cells resembling delta cells also constitute a small population. High correlation is observed between the same cell type at different time points, both in absolute (r²>0.8) and relative terms, as compared to other cell types from any time point (FIG. 11F). Importantly, for endocrine cells, no evidence is seen of dedifferentiation toward a progenitor state nor transdifferentiation toward alternative fates during Stage 6. It was thus concluded that the global transcriptional profiles, serving as measure of identity, are maintained during extended Stage 6 culture.

Consistent with their glucose responsiveness, it is observed that SC-beta cells express key genes of beta cell identity¹⁵, metabolic sensing and signaling¹⁶ and insulin synthesis, packaging and secretion¹⁷. Broadly, these genes are expressed in both cadaveric islet beta cells and SC-beta cells but not in the NKX6.1+ progenitors of the later (FIGS. 17C-17F). There appears to be minimal cell replication as evidenced by the negligible expression of cell-cycle associated genes (TOP2A) and high expression of the cell cycle inhibitor CDKN1C.

Finally, it is sought to describe the refinements in SC-beta gene expression that occur over time. Pseudotime analysis was applied to order the cells according to their transcriptional state and regressed gene expression using pseudotime to identify dynamic genes (FIGS. 11G-11H). Genes increasing along pseudotime include IAPP and other markers of beta cell maturity such as HOPX¹³, NEFM¹⁸ and SIX2^{13,18} (FIG. 11I), although some markers of maturity or age (UCN3¹⁹, MAFA¹⁸ and SIX3¹⁸) were not expressed. Decreasing genes include LDHA, whose suppression is necessary for proper metabolic sensing²⁰, and IGF2, a secreted peptide downstream of the INS gene, suggesting better transcriptional regulation of insulin's genomic locus. In summary, relatively subtle changes are observed in SC-beta transcriptomes during Stage 6, some of which correspond to known markers of maturation.

### Early SC-Alpha Cells Express Insulin

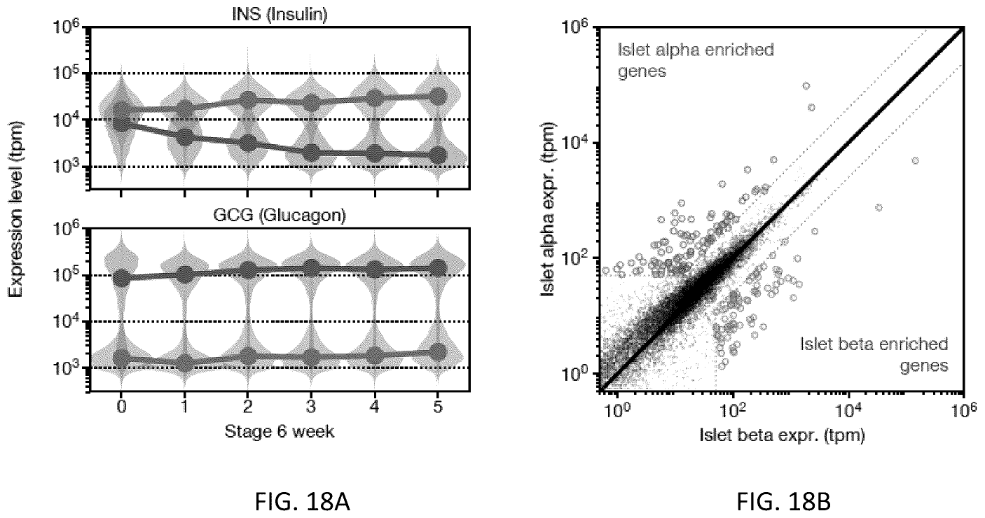

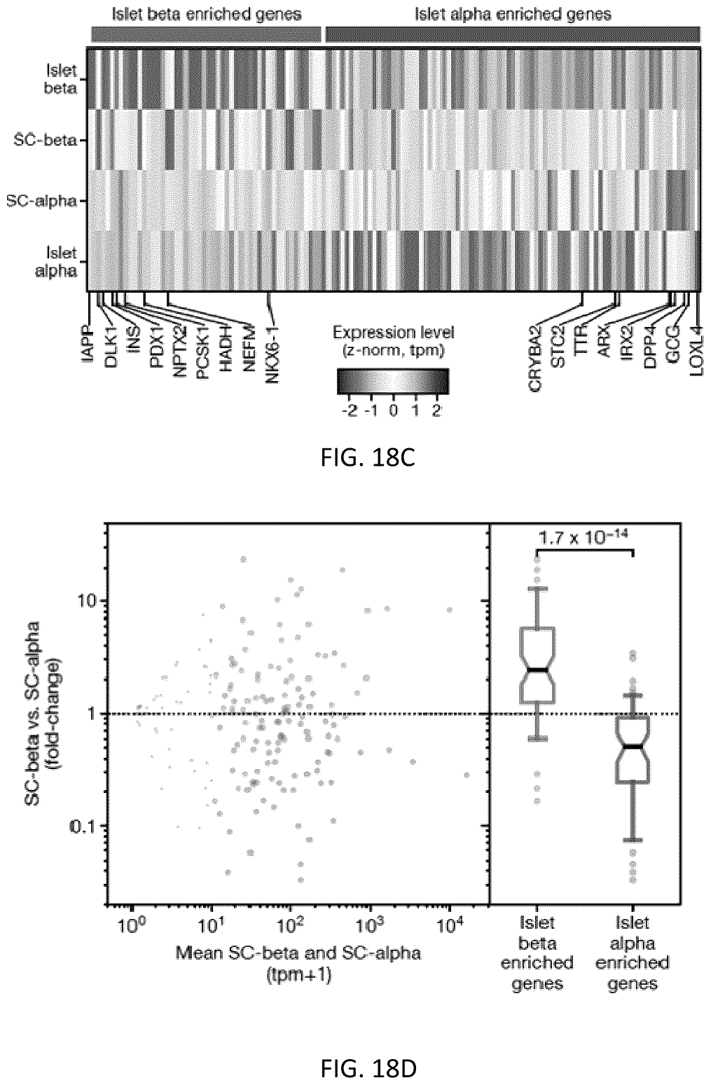

Poly-hormonal cells, expressing both insulin and glucagon, have been reported in several in vitro pancreatic differentiation protocols. Beyond glucagon, these cells express many markers of islet alpha cells, but uncharacteristically express insulin. On this basis, and because expression of insulin is rectified during Stage 6 (FIG. 18A), these cells are referred to as SC-alpha cells. To explore the similarity of SC-alpha and SC-beta cells to their in vivo counterparts, genes differentially expressed between adult cadaveric alpha and beta cells were identified⁵ (FIG. 18B). Genes with higher expression in alpha cells were higher in SC-alpha cells whereas beta cell-enriched genes were higher in SC-beta cells (FIGS. 18C-18D). This result is consistent with previous findings that in vitro-derived poly-hormonal cells resolve to mono-hormonal glucagon-expressing cells²¹. Cells co-expressing insulin and glucagon have been observed in two contexts: human fetal pancreatic development, where INS+/GCG+/ARX+ cells are described as alpha precursors²², and in Type 2 Diabetes, where INS+/GCG+ cells are described as dedifferentiated beta cells²³. Given the evidence that they are a transient state toward mono-hormonal SC-alpha cells, in vitro poly-hormonal cells are more likely to match the developmental INS+/GCG+/ARX+cells.

### Stem-Cell Derived Enterochromaffin Cells

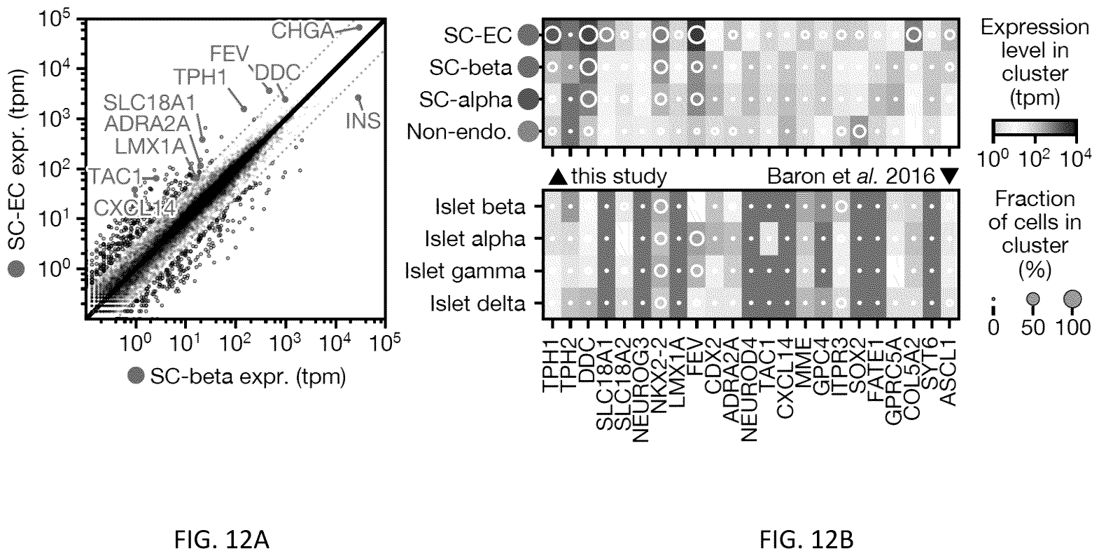

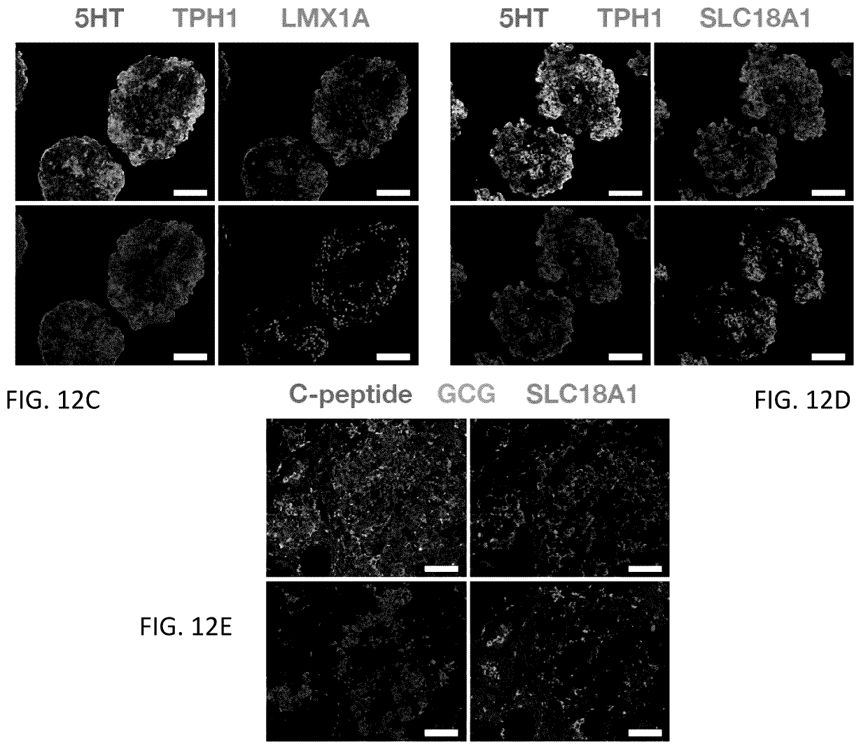

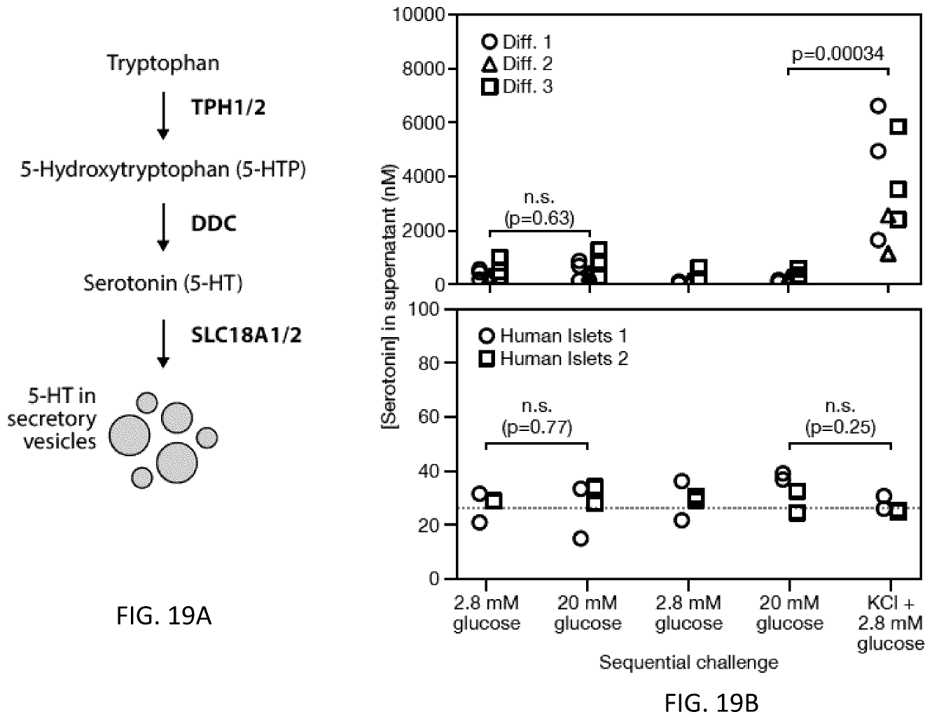

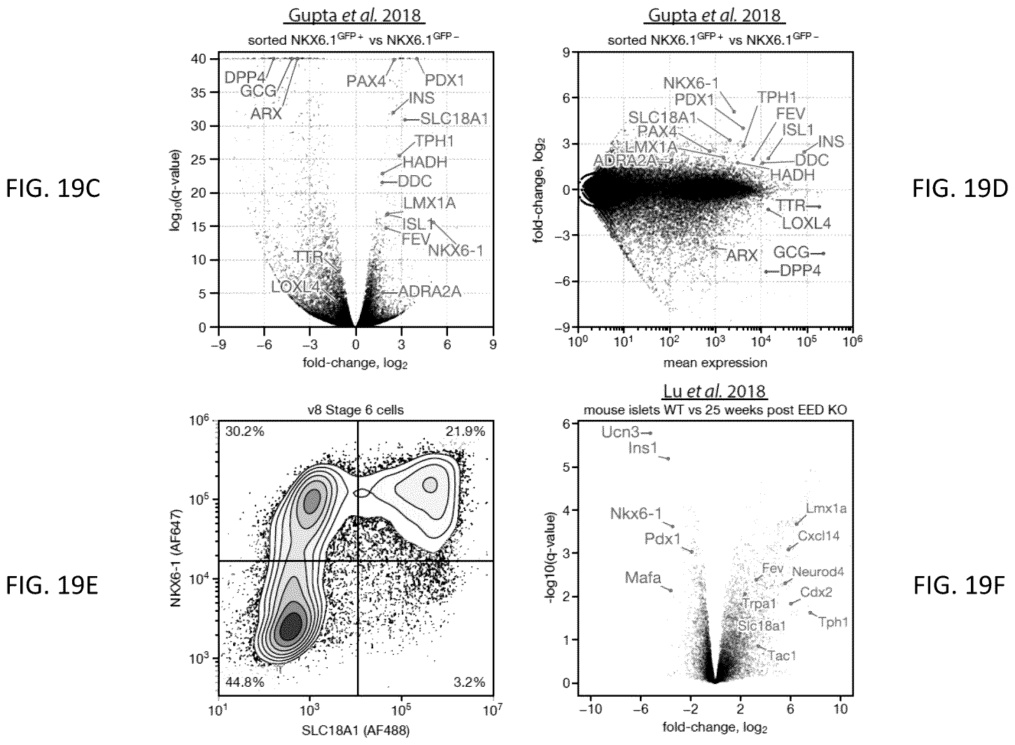

This survey identified a population of endocrine cells expressing TPH1, NKX6.1 and low levels of insulin, but lacking beta cell markers G6PC2, NPTX2, ISL1 and PDX1. It is hypothesized that these cells are stem-cell derived enterochromaffin cells (SC-EC). Enterochromaffin cells synthesize and secrete serotonin (5-HT) in the gut where they serve as chemosensors²⁴. Their transcriptome has been characterized via single-cell sequencing of murine intestinal epithelium²⁵ and organoids²⁶. Compared to SC-beta cells (FIG. 12A), SC-EC cells express genes required for serotonin synthesis (TPH1, DDC, SLC18A1, FIG. 19A), and markers such as LMX1A, ADRα2A, FEV, TAC1 and CXCL14. The expression of these genes is enriched in SC-EC cells relative to both other in vitro populations, and in vivo pancreatic populations (FIG. 12B). By immunostaining (FIGS. 12C-12D), it is verified that SC-EC cells co-express TPH1, LMX1A and SLC18A1 and contain serotonin (5-HT). Like SC-beta cells, these cells survive transplantation in the kidney capsule of mice (FIG. 12E). SC-islets release serotonin upon depolarization with KCl, but not upon stimulation with high glucose (FIG. 19B), consistent with the expected behaviors of EC cells²⁷. SC-EC cells are observed in all datasets of this study. Also observed is expression of SC-EC genes in bulk expression data²⁸ from iPSC differentiations using a different protocol (FIGS. 19C-19E), suggesting the presence of EC cells across other beta cell protocols and pluripotent cell lines.

Although serotonin is reportedly produced in human beta cells²⁹, expression of TPH1 is not observed in either in vivo or in vitro beta populations⁵⁻⁹, nor are EC cells found in single cell profiling of the pancreas⁵⁻¹¹. Other studies have shown that beta cells produce serotonin in age- or context-dependent manners, not explored in existing single-cell datasets²⁹⁻³¹. However, a signal of the induction of a serotonin/EC program in perturbed mouse beta cells was identified from recently published data³², suggesting a small “distance” between the beta and EC fates. Specifically, 25 weeks after a beta-cell specific knockout of the Polycomb repressive complex 2 (PRC2) component EED, upregulation of enterochromaffin marker genes Tph1, Lmx1a, Slc18a1 and Trpa1 is noted (FIG. 19F). This analysis shows that the serotonin/EC program is induced in a model of beta cell dedifferentiation, suggesting a relationship between the beta and EC fates.

### Fates of Non-Endocrine Cells

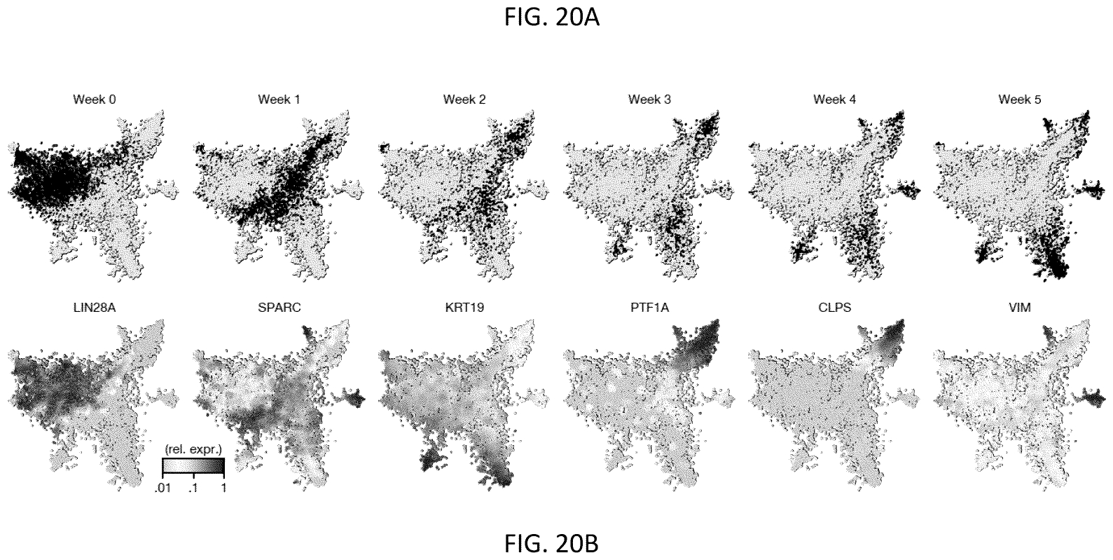

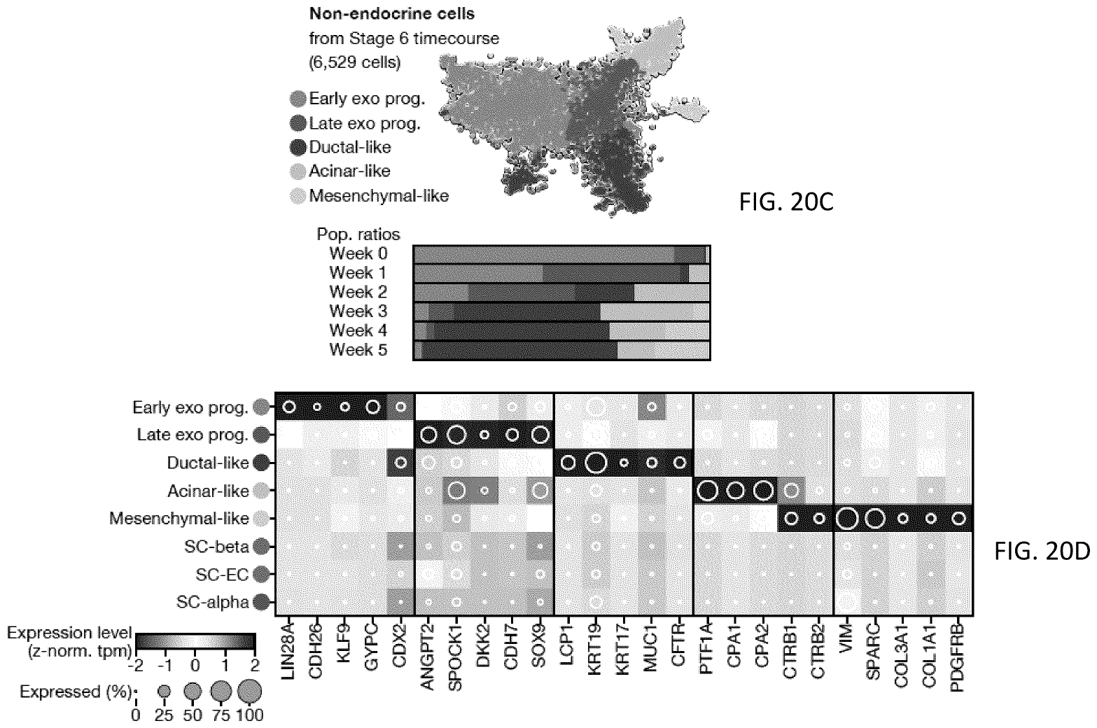

Some cells do not adopt an endocrine fate during Stages 4 and 5 (FIG. 20). These non-endocrine cells are similar to pancreatic progenitor cell types from earlier stages in their expression of key transcription factors and lack of endocrine markers. Whereas both in vivo and in vitro endocrine cells are largely post-mitotic, these non-endocrine cells retain expression of cell cycle associated genes (TOP2A, FIG. 23). These cells do not follow endocrine commitment, nor do they remain as progenitors and instead appear to differentiate toward exocrine pancreatic fates. During continued culture in Stage 6, they split into populations that express markers of pancreatic acinar, mesenchymal and ductal cells (FIG. 20).

### Purification of Endocrine and SC-Beta Cells

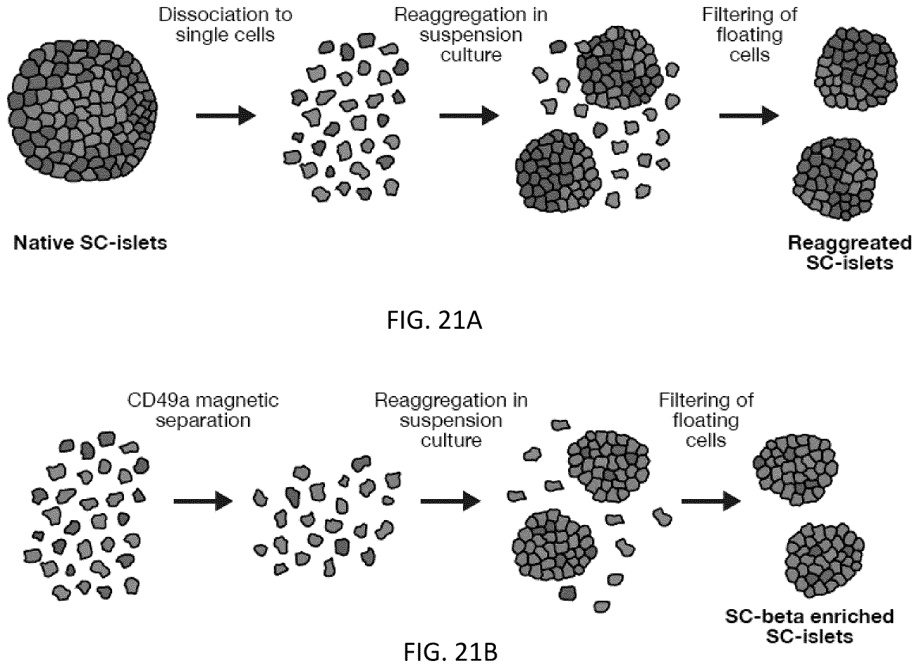

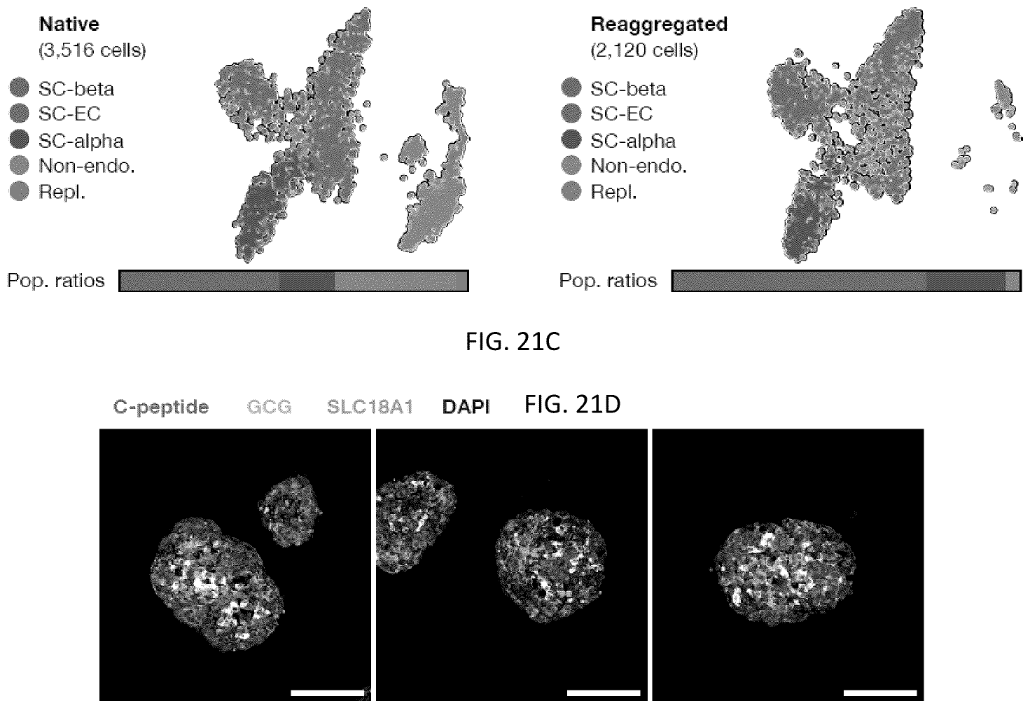

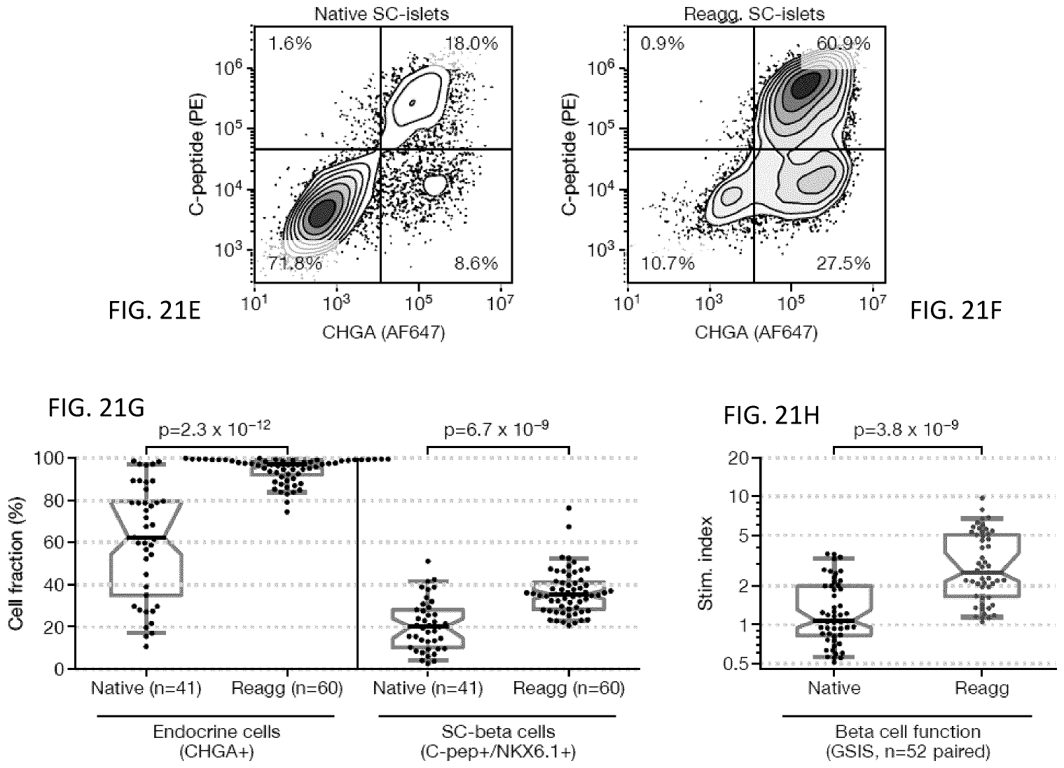

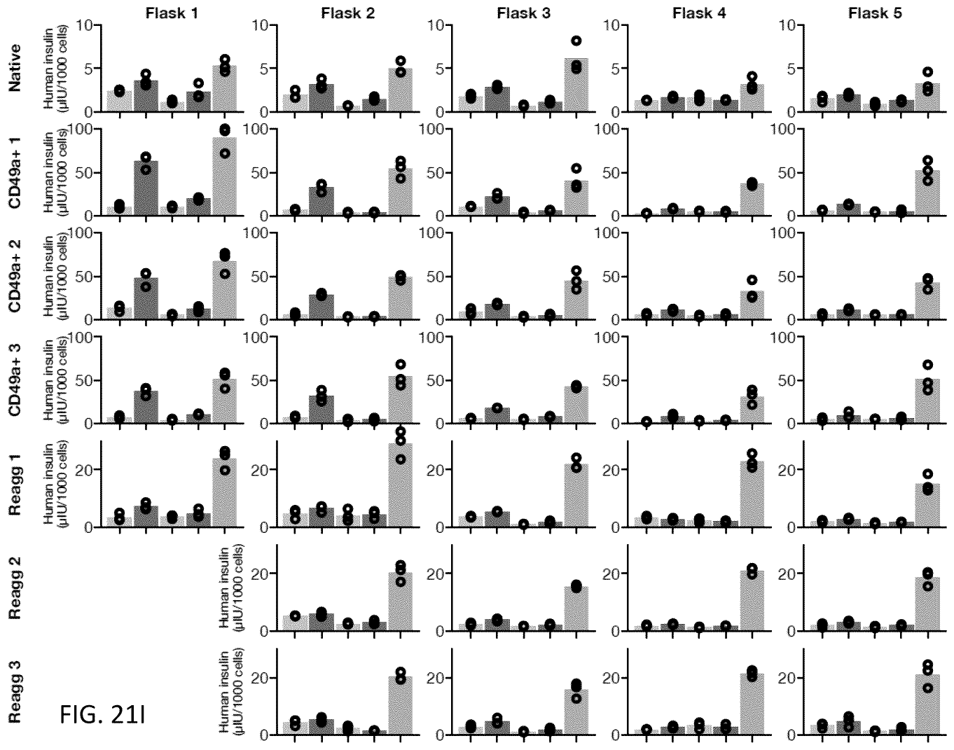

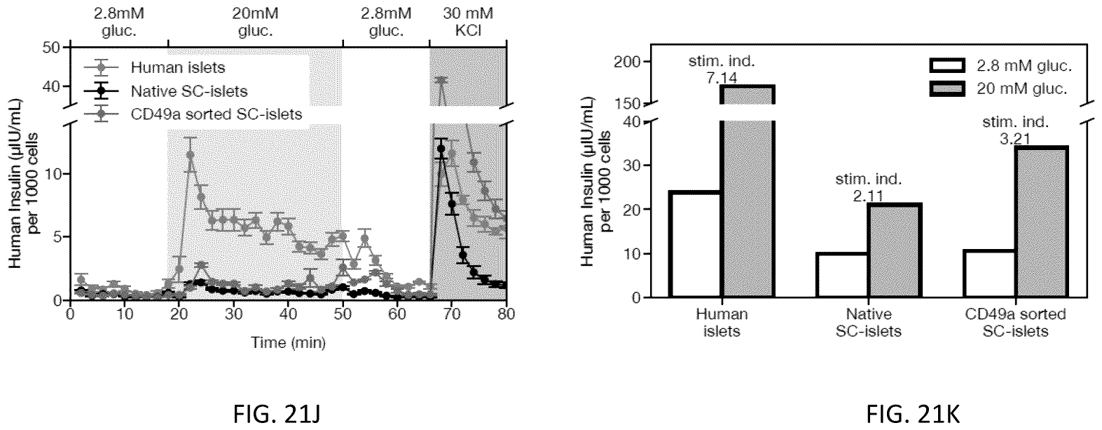

Single-cell dissociation followed by controlled re-aggregation has been used to purify endocrine cells from neonatal pancreas³³ and in vitro beta cell preparations³⁴. It was discovered that enzymatic dissociation followed by re-aggregation can be applied after Stage 5. Unlike previous methods, this approach is scalable because it does not require micro-patterned surfaces, hanging droplets or soluble extracellular matrix factors to increase efficiency. Using single-cell sequencing, flow cytometry and GSIS (FIGS. 21A-21H), its shown that this re-aggregation procedure depletes non-endocrine cells while maintaining cell identity and improving beta cell function.

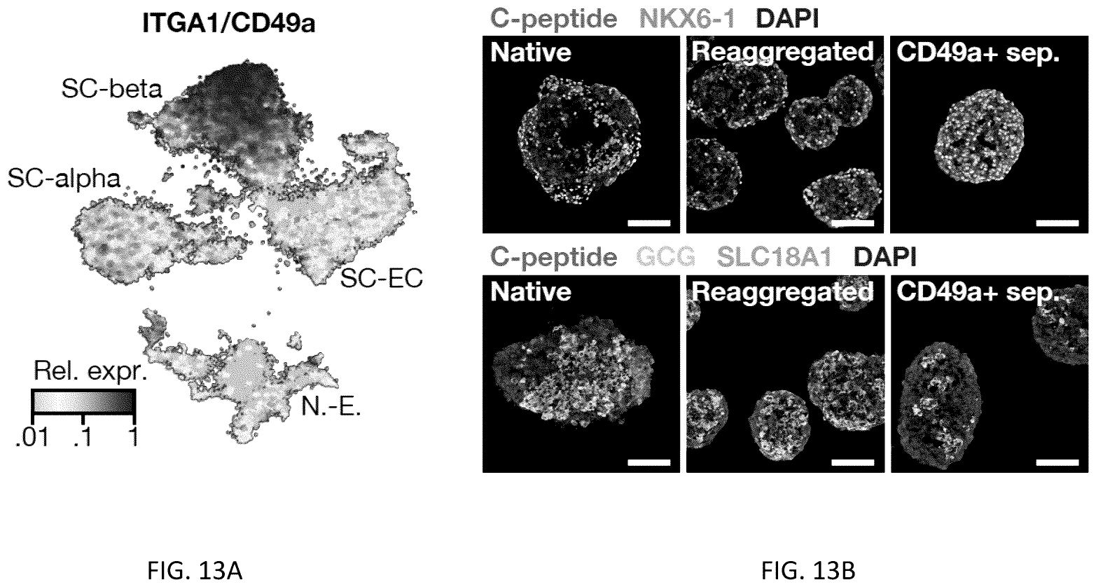

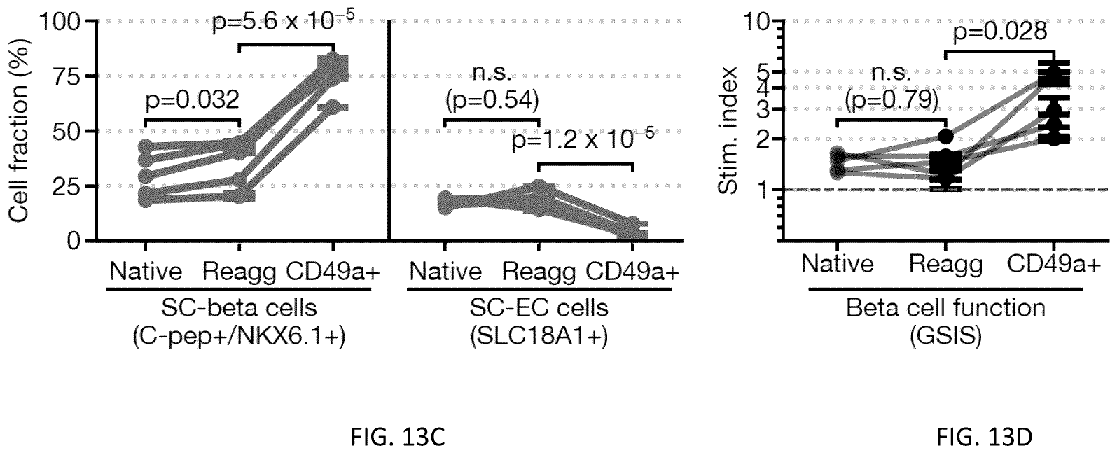

Interestingly, staining of SC-islets after re-aggregation shows marked compartmentalization of endocrine cell populations into regions of like cells. Beyond endocrine enrichment, ways of specifically enriching for SC-beta cells were explored. The analysis identifies ITGA1 (CD49a) as a novel SC-beta surface marker (FIG. 13A). Interestingly, within the adult islet ITGA1 expression is not specific to beta cells⁵. Anti-CD49a staining and magnetic microbeads were used to label and efficiently sort SC-beta cells. This method produces clusters containing up to 80% SC-beta cells (FIGS. 13B-13C), with fewer than 5% SC-EC cells. Comparable purification from differentiations of an additional ESC and two iPSC lines is observed (data not shown). These highly purified SC-islets are responsive to glucose in vitro (FIG. 13D, FIGS. 21I-21K), with increased stimulation indices compared to unsorted, re-aggregated SC-islets in both static and dynamic GSIS, but lower secretion magnitude compared to cadaveric islets in both. Thus, the single cell sequencing data has revealed a novel approach for enriching beta cells produced in vitro.

### The Origin and Lineage of SC-Beta Cells

Single cell sequencing can reconstruct complex developmental trajectories both from single snapshots or sequential samplings. SC-beta and SC-EC cells are absent at the end of Stage 4 and appear during the course of Stage 5. Given shared expression of key genes (such as PAX4, NKX6-1), it was sought to determine whether these cells form separately during endocrine induction or whether one is a precursor for the other. To this end, ˜45,000 cells were sequenced at daily intervals throughout the course of Stage 5 for two independent differentiations.

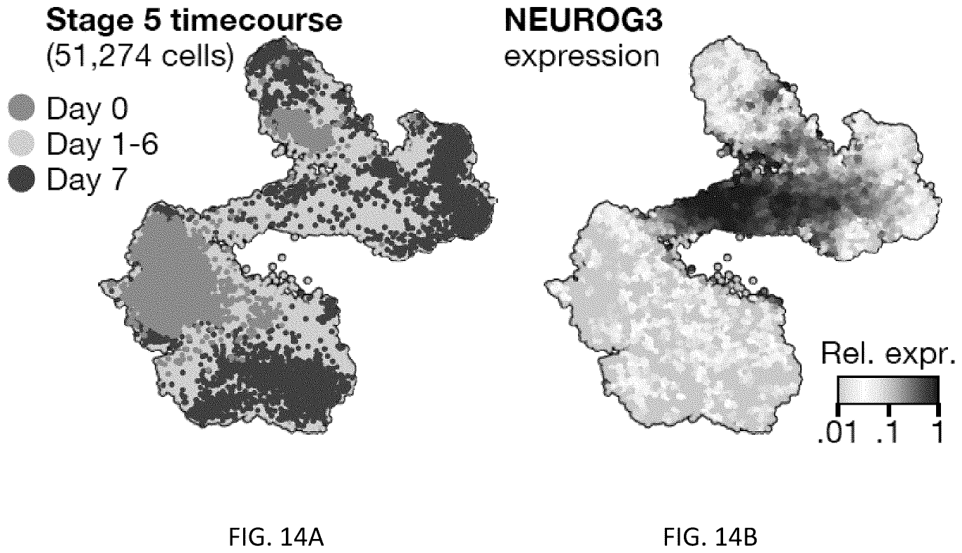

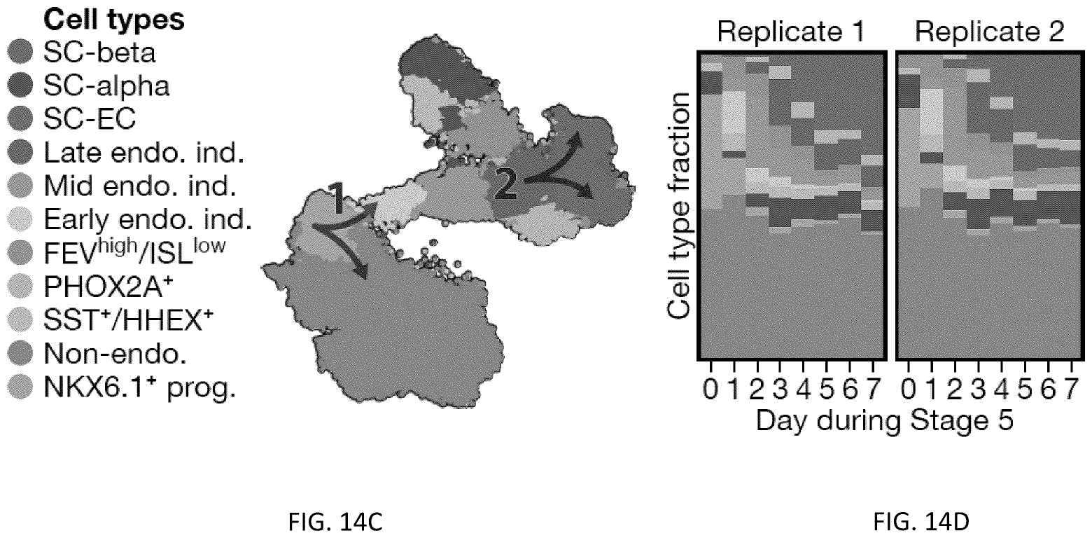

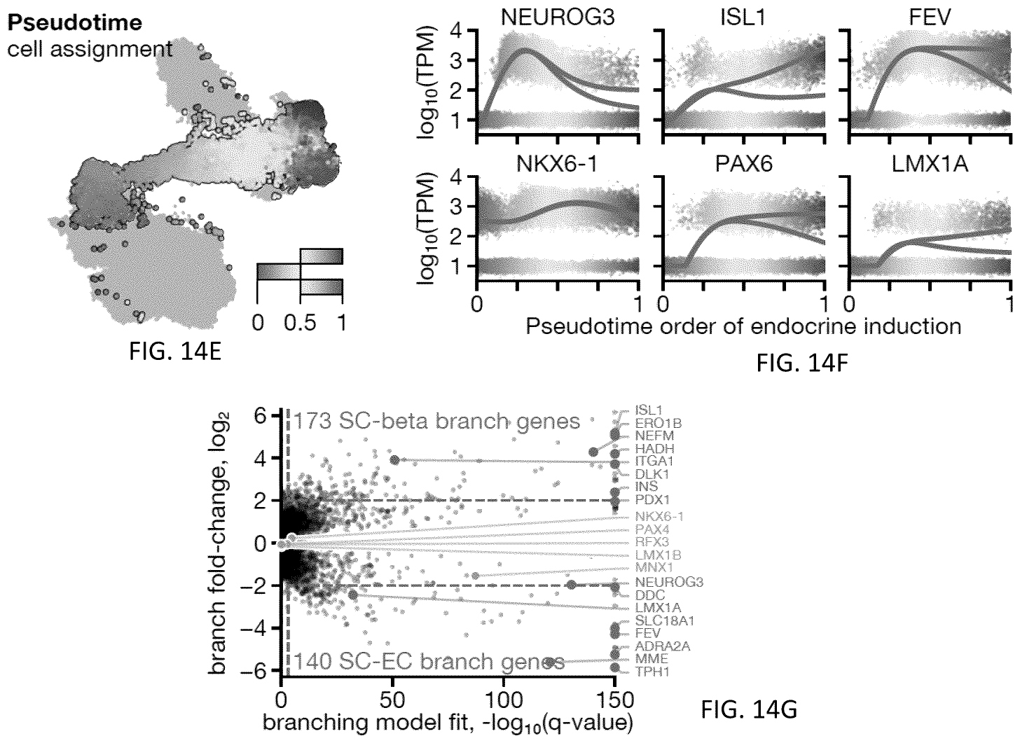

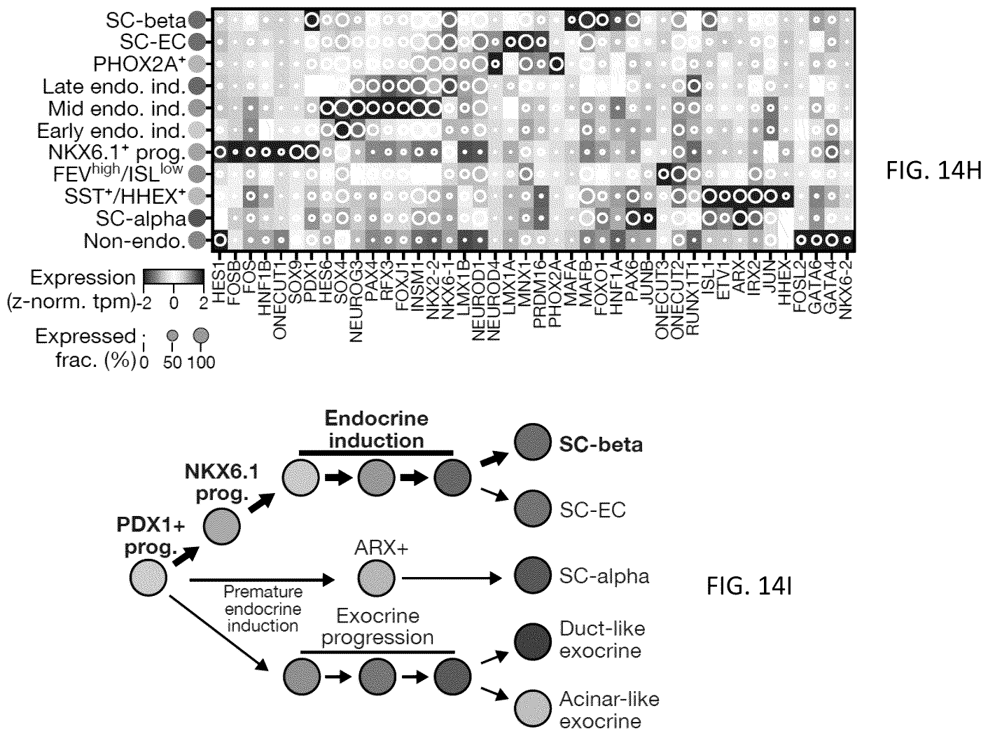

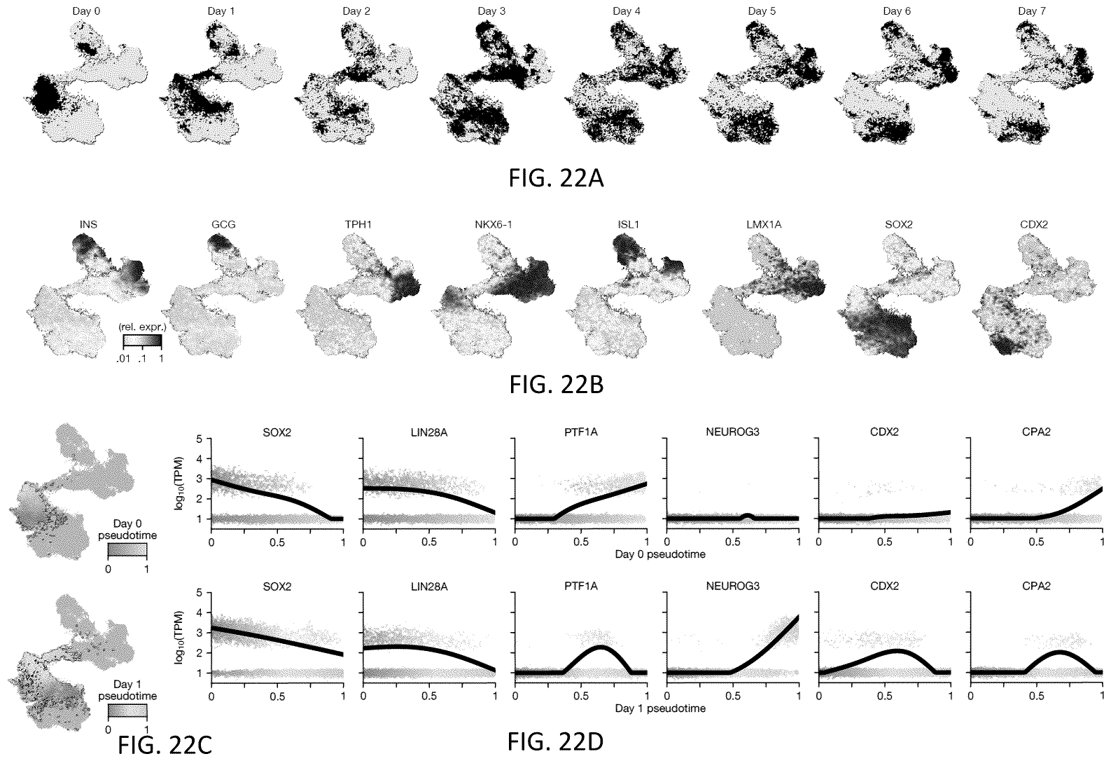

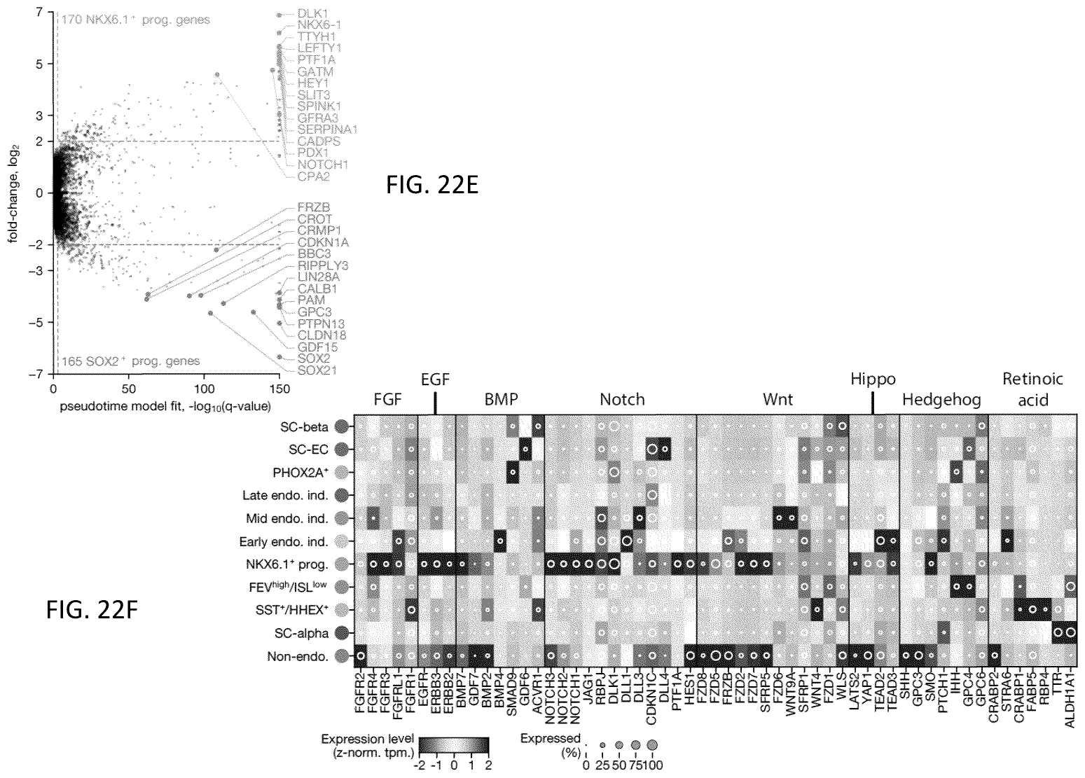

From a global perspective, individual cells in this dataset form a continuum connecting Stage 5 day 0 and day 7 populations. NEUROG3, a transiently-expressed master regulator of in vivo endocrine induction, is expressed by cells bridging endocrine and non-endocrine cells within this continuum as different cell types gradually emerge (FIGS. 14A-14D, FIG. 14H, FIGS. 22A-22B). Some day 0 cells are already endocrine, matching either SC-alpha cells (ARX+), or delta-like cells showing co-expression of SST and HHEX. Other day 0 cells (marked by FEV+/ISL− but NEUROG3−) resemble NEUROG3+ cells from later timepoints and likely represent partial endocrine induction. The trajectory that connects progenitors to SC-beta cells contains two bifurcation events that are explored (arrows in FIG. 14C).

The initiation of endocrine induction is the first major bifurcation of cells during Stage 5. On day 0, progenitors form a single heterogenous population characterized by a gradient from SOX2+, FRZB+, PDX1^{low} to NKX6.1+, PTF1A+, PDX1^{high} cells (FIGS. 22C-22E). Pseudotime ordering of these progenitors identifies 335 genes correlated with the gradient. On day 1, NEUROG3+ expression is observed at the NKX6.1+, PTF1A+, PDX1^{high} end of the gradient, and thus it is inferred that these genes mark progenitors most poised for endocrine induction. NEUROG3 expression is accompanied by changes in many other transcription factors and cellular signaling genes (FIG. 22F). Also observed, starting on day 1, is an upregulation of CDX2 (FIG. 22B, FIG. 22D) among a subset of the NKX6-1+ cells that have yet to or fail to undergo endocrine induction. The analysis reveals an axis of Stage 4 progenitor variation, marked by NKX6.1+, PTF1A+ and PDX1^{high} that predicts endocrine induction potential.

Stage 5 endocrine induction primarily yields SC-beta and SC-EC cells, with the earliest cells of these types emerging on day 3. Global clustering and manifold embedding suggest a late branching of the SC-beta and SC-EC fates. To validate this branching observation, diffusion pseudotime of all SC-beta, SC-EC and NEUROG3+ cells was computed (FIGS. 14E-14G). Fitted to each gene is a model incorporating both pseudotime and branch assignment as covariates and these models are compared to ones fit without branch labels. While some genes (like NEUROG3 and NKX6.1) are dynamically expressed but show no, or little, branch dependence (FIG. 14F), 313 branch-associated genes are identified (q-val <0.001 and fold-change >4), including many transcription factors and key SC-beta and SC-EC fate genes. The analysis suggests that SC-beta and SC-EC cells emerge from a common NEUROG3+ induction intermediate, rather than one serving as a progenitor for the other. Thus, this constitutes a second fate bifurcation on the trajectory of SC-beta formation. From this analysis, a model is proposed for the lineage of cell types produced by SC-beta differentiation (FIG. 14I).

### Discussion

Beta cells are front-runners in the field of regenerative medicine. Nonetheless, directed differentiation protocols for beta cells produce other cells alongside them. In this study, single-cell RNA sequencing experiments are used to comprehensively characterize cells formed during SC-beta differentiation.

The stepwise, synchronous differentiation of millions of cells provides an unprecedented opportunity to study human developmental processes. It is shown that SC-beta cells respond to glucose in vitro and maintain their identity under extended culture without signaling modulators. Dynamic genes include several markers of beta cell maturation. Furthermore, the identity of poly-hormonal cells has previously been controversial. It is concluded that they represent alpha-like (SC-alpha) cells that only transiently misexpress insulin. In the context of transplantation, these cells may improve beta cell function through local interactions or autocrine signaling within SC-islets. It is shown that progenitors that fail endocrine induction progress toward pancreatic exocrine cell types. These seem undesirable, as they may replicate or occupy precious space within transplantation devices. To eliminate them, a scalable re-aggregation method is described that enriches endocrine cells. Additionally, CD49a is identified as a surface marker of SC-beta cells and highly pure SC-beta clusters are generated via magnetic sorting.

An unexpected finding of this analysis is the existence of SC-EC cells in vitro. It is shown that SC-EC cells are closely related but fundamentally distinct from SC-beta cells, arising from a late bifurcation of differentiation. Given this close similarity and their expression profile for key genes (NKX6.1+/CHGA+/GCG−), these cells may be misclassified as either progenitors or bona fide beta cells when analyzed via methods using preselected groups of genes¹⁴. In vivo, enterochromaffin cells have not been observed in studies of mouse and human islets⁵⁻⁹. Nonetheless, extremely rare reports of primary pancreatic serotonin-producing carcinoid tumors support the existence resident pancreatic enterochromaffin cells³⁵. Importantly, it is shown that CD49a purification depletes SC-EC cells.

This study provides a resource for future development of beta cell differentiation protocols. For instance, hypotheses on controlling cell fate by modulating signaling pathways may be guided by receptor expression patterns or inferred signaling activities. Although SC-beta cells are highly similar to cadaveric beta cells, differences remain including the lack of expression of UCN3, MAFA, and SIX3. While these genes are likely expressed after transplantation in vivo, they represent the next milestone in the pursuit of ever more mature SC-beta cells in vitro. In parallel, further milestones in characterizing SC-beta differentiation will come from single-cell measurements of proteins, epigenetics and lineage.

Overall, a comprehensive and detailed analysis is provided of a stem-cell product destined for human therapeutics. This type of high-resolution, single-cell profiling represents a necessary step on the road toward successful and safe therapies.

### Methods

### Cell Culture

Human pluripotent stem cell (hPSC) maintenance and differentiation was carried out as previously described¹. Pluripotent stem cell lines were obtained from stocks maintained by the Melton lab or Semma Therapeutics. Lines were identified by DNA fingerprinting (Cell Line Genetics) and all lines tested negative on routine mycoplasma contamination verifications. Pluripotent stem cell lines were maintained in cluster suspension culture format using mTeSR1 (Stem Cell Technologies, 85850) in 500 mL spinner flasks (Corning, VWR) spinning at 70 rpm in an incubator at 37° C., 5% CO₂ and 100% humidity. Cells were passaged every 72 hours: hPSC clusters were dissociated to single cells using Accutase (Innovative Cell Technologies; AT104-500) and light mechanical disruption, counted, and seeded at 0.5 M cells/mL in mTeSR1+10 μM Y27632 (DNSK International, DNSK-KI-15-02).

Differentiation flasks were started 72 hours after passage by removing mTeSR1 media and replacing with the protocol-appropriate media and growth factor or small molecule supplements (see FIG. 26). Small molecules and signaling factors are prepared and stored as single use aliquots. During feeds, the differentiating clusters are allowed to gravity settle for 5-10 minutes, media is aspirated, and 300 mL of pre-warmed media is added. All experiments involving human cells were approved by the Harvard University IRB and ESCRO committees.

### Flow Cytometry

Differentiated clusters, sampled from the suspension culture (1-2 mL), were dissociated using TrypLE Express (Gibco; 12604013) at 37° C., mechanically disrupted to form single cells, fixed using 4% PFA for 30 minutes at RT and stored in PBS at 4° C. For staining, fixed single cells were incubated in blocking buffer for 1 hour at RT, then incubated in blocking buffer with primary antibodies (1 hr at RT or overnight at 4° C.), washed three times with blocking buffer, incubated with secondary antibodies in blocking solution (1 hr at RT), washed three times and resuspended in PBS+0.5% BSA (Proliant; 68700). Blocking buffer: PBS+0.1% saponin (Sigma; 47036)+5% donkey serum (Jackson Labs; 100181-234). Stained cells were analyzed using the LSR-II, Accuri C6 (BD Biosciences) or Attune NxT (Invitrogen) flow cytometers. An example gating strategy is shown in FIG. 22. Results presented in this study are representative of more than a hundred independent v8 differentiations.

Immunofluorescence Microscopy Differentiated clusters were fixed in 4% PFA for 1 hour at RT, washed and frozen in OCT and sectioned. Prior to staining, paraffin-embedded samples were treated with Histo-Clear to remove the paraffin. All slides were rehydrated via an ethanol gradient and incubated in boiling antigen retrieval reagent (10 mM sodium citrate, pH 6.0) for 30 minutes. For staining, slides were incubated in CAS block (ThermoFisher; 008120) with primary antibody overnight at 4° C., washed three time, incubated in secondary antibody for 2 hours at RT, washed, mounted in Vectashield with DAPI (Vector Laboratories; H-1200) or ProLong Diamond Antifade Mountant with DAPI, covered with coverslips and sealed with clear nail polish. Representative regions were imaged using Zeiss.Z2 with Apotome or Zeiss CellDiscoverer 7 microscopes. Images shown are representative of similar results in at least 3 biologically separate differentiations from matched or similar stages.

### Antibodies

Primary antibodies (supplier; catalog number, effective dilution): rat anti-C-peptide (DHSB; GN-ID4; 1:100), mouse anti-NKX6.1 (DHSB; F55A12; 1:50), rabbit anti-CHGA (Abeam; ab15160; 1:500), rabbit anti-SLC18A1 (Sigma; HPA063797; 1:300), rabbit anti-LMX1A (Sigma; HPA030088; 1:300), sheep anti-TPH1 (EMD Millipore; AB1541; 1:100), goat anti-5-HT (Immunostar; 20079; 1:1000), rabbit anti-SOX9 (Cell Marque; AC-0284RUO; 1:500), mouse anti-glucagon (Santa Cruz Biotech.; SC-514592; 1:300).

Secondary antibodies (supplier; catalog number, all used at 1:300 dilution): anti-rat 594 (Life Tech.; A21209), anti-mouse 594 (Life Tech.; A21203), anti-mouse 647 (Life Tech.; A31571), anti-rabbit 488 (Life Tech.; A21206), anti-rabbit 594 (Life Tech.; A21209), anti-rabbit 647 (Life Tech.; A31573), anti-goat 647 (Life Tech.; A21447), anti-sheep 488 (Life Tech.; A1015), anti-rat 488 (Jackson labs.; 712-546-153), Anti-rat 405 (Abcam; ab175670).

### Transplantation Studies

Transplantation of differentiated clusters was carried out as previously described¹. Briefly, ˜500 IEQ human islets or ˜5×10⁶ Stage 6 native (day 10, non-reaggregated)SC-islet clusters were transplanted under the kidney capsule of male SCID beige mice (Jackson labs) aged between 8 and 12 weeks. At the specified time after transplantation, kidneys containing grafts were dissected and fixed in 4% PFA overnight at 4° C. The fixed kidneys were embedded in paraffin and sectioned for immunofluorescence staining, which was performed as described above. All animal studies were approved by the Harvard University IACUC.

### Glucose Stimulated Insulin and Serotonin Secretion

Human islets (˜400 IEQ, Prodo Laboratories) or SC-islet clusters (equivalent to ˜4×10⁶ cells between 28 and 60 days of differentiation) were divided into four parts to collect technical triplicate and insulin/serotonin content samples. Krebs buffer (KRB) was prepared: 128 mM NaCl, 5 mM KCl, 2.7 mM CaCl₂), 1.2 mM MgSO₄, 1 mM Na₂HPO₄, 1.2 mM KH₂PO₄, 5 mM NaHCO₃, 10 mM HEPES (Life Technologies; 15630080), 0.1% BSA in deionized water. Clusters were washed twice with low-glucose (2.8 mM) KRB and were then loaded into the 24 well plate inserts (Millicell Cell Culture Insert; PIXP01250) and fasted in low-glucose KRB for 1 hr to remove residual insulin in 37° C. incubators. Clusters were washed once in low-glucose KRB, incubated in low-glucose KRB for 1 hour, and supernatant collected. Then clusters were transferred to high-glucose (20 mM) KRB for 1 hour, and supernatant collected. This sequence was repeated one additional time and clusters were washed once between high-glucose to second low-glucose incubation to remove residual glucose. Finally, clusters were incubated in KRB containing 2.8 mM glucose and 30 mM KCl (depolarization challenge) for 1 hour and then supernatant collected. Clusters were then dispersed into single cells using TrypLE Express, and cell number was counted automatically by a Vi-Cell (Beckman Coulter) to normalize insulin level by the cell number. Supernatant samples containing secreted insulin were processed using the Human Ultrasensitive Insulin ELISA (ALPCO, 80-INSHUU-EO1.1) and the Serotonin ELISA (ALPCO; 17-SERHU-E01-FST).

### Dynamic Perifusion Assay for Glucose Stimulated Insulin Secretion

Dynamic GSIS was performed as previously described¹⁹. Non-diabetic human islets from Prodolabs (100-250 um diameter sized 25 IEQ islets were handpicked per sample, n=3) and native or purified SC-beta clusters (100-250 μm diameter sized 25 clusters were handpicked per sample, n=3), were assayed on a fully automated Perifusion System (BioRep). Chambers were sequentially perifused with 2.8 mM or 20 mM glucose, or 2.8 mM glucose with 30 mM KCL in KRB buffer at a flow rate of 100 ul/min. Chambers were first perifused with low glucose (2.8 mM) for 1 hour for fasting and then 15 minutes for low glucose incubation followed by high glucose (20 mM) challenge for 30 minutes. Samples were then perifused with low glucose for 15 minutes followed by low glucose and 30 mM KCl for 15 minutes. Insulin concentrations in the supernatant were determined using an Ultrasensitive Insulin ELISA kit (Alpco; 80-INSHUU). The insulin secretion levels were normalized by total cell number (uIU/mL/1000 cells).

### Re-Aggregation Procedure to Remove Non-Endocrine Cells

The re-aggregation procedure was optimized for scalability, in order to ensure that the method (unlike previous related techniques^{34,36-39}) may be deployed at scales of several billion cells. SC-islets were dissociated into single cells at the end of Stage 5 differentiation. 300 mL of SC-islets culture were washed in PBS and incubated in 25 mL of TrypLE Express for 20 min at 37° C. Cells were then quenched with DMEM+10% FBS and spun down, before resuspending in 10 mL of Stage 6 culture media. Remaining undissociated cell clusters were mechanically dissociated using a P1000 pipette. The single cell suspension is further diluted to a volume of 50 mL with Stage 6 media, before being passed through a 40 μm mesh filter (pluriSelect) to remove any residual undissociated clusters. The dissociated single cells were counted and seeded into a spinner flask at a density of 1M cells/mL in Stage 6 media and cultured in an incubator at 37° C. with 70 rpm agitation. The endocrine cells self-aggregate into clusters within 24 hours, while progenitor cells remain in the supernatant. After 48 hours of culture, cells were fed by spinning down all the cells and resuspending in fresh Stage 6 media. Subsequent media changes were done every 48 hours using a 20 μm mesh filter (pluriSelect). The re-aggregated clusters enriched with endocrine cells were collected on the 20 μm mesh filter and reseeded back in the spinner flask with Stage 6 media at the original volume. Supernatant containing single cells that passed through the 20 μm mesh filter were discarded.

### Magnetic Enrichment Using CD49a/ITGA1

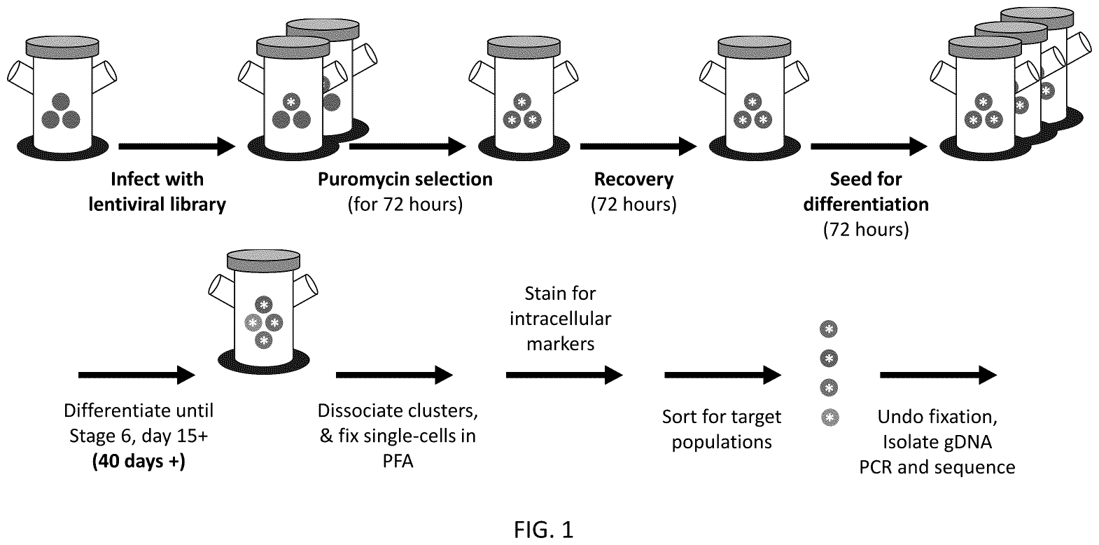

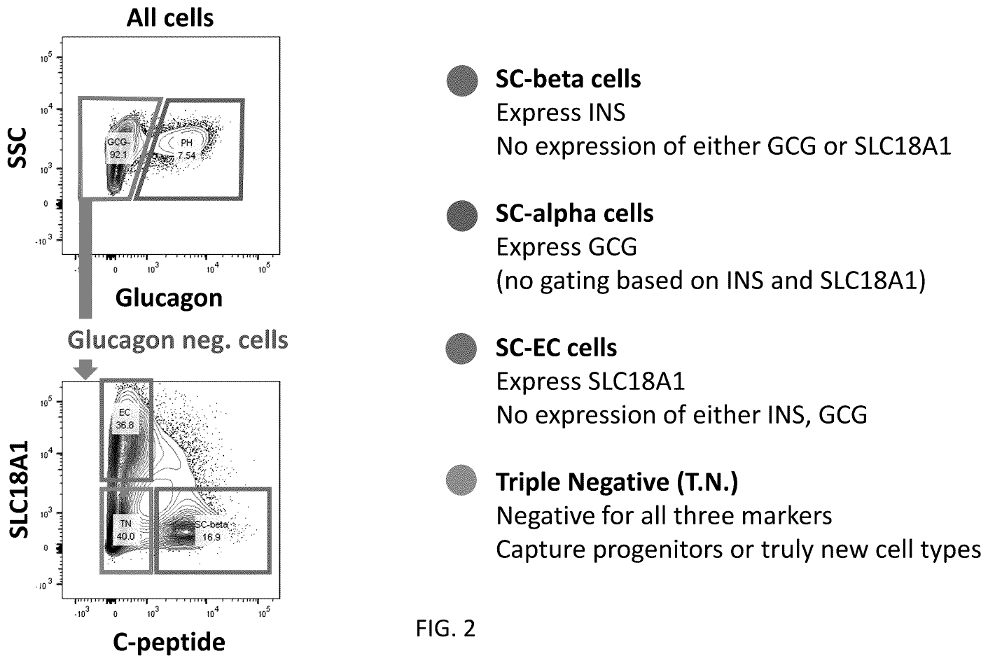

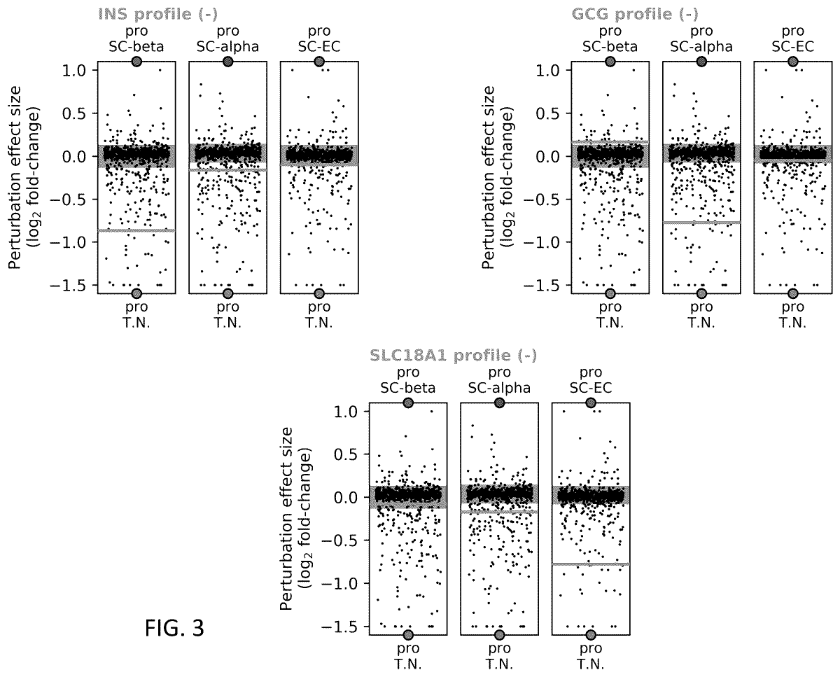

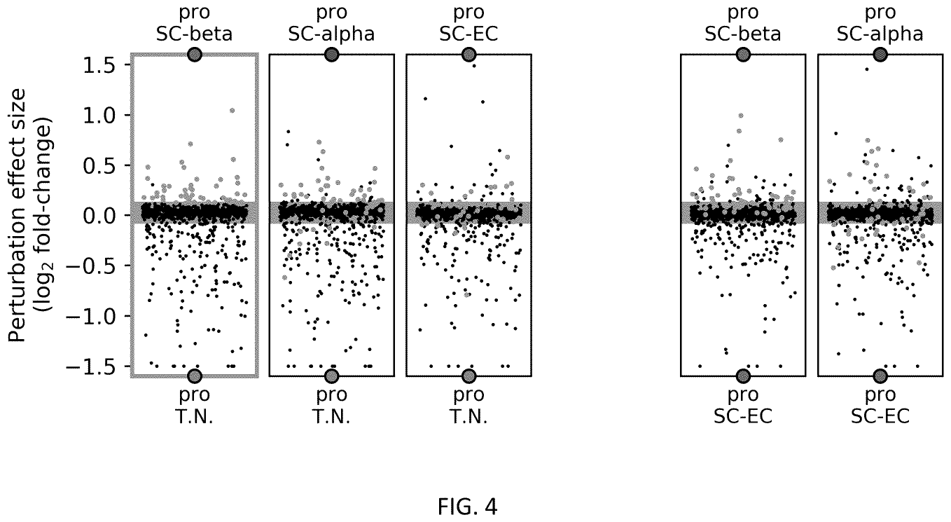

Stage 6 clusters (taken at Stage 6 week 2) were dissociated as in the re-aggregation section above, starting with 75 mL of Stage 6 culture. The dissociated single-cells were resuspended in sorting buffer (PBS+1% BSA+2 mM EDTA) and filtered through a 35 μm mesh filter. Cells were counted and resuspended at a density of 10M cells per 300 μL in 15 mL conical tubes. Cells were stained at room temp for 20 minutes using a 1:100 dilution of Anti-human CD49a PE-conjugated (BD #559596) antibody, covered from light and agitated every 3 minutes. Stained cells were washed twice with 15 mL of sorting buffer by spinning down (5 min, 300 g) and resuspending to their initial density of 10M cells per 300 μL. To label with microbeads, 40 μL of anti-PE UltraPure MACS microbreads (Miltenyi 130-105-639) were added for each 1GM cells and the cell solution was incubated for 15 minutes at 4° C., agitated every 5 minutes. The stained cells were washed twice as above and resuspended to a target density of 25M-30M cells per 500 μL. Volumes of 500 μL (containing no more than 30M cells) were then magnetically separated on LS columns (Miltenyi 130-042-401) in a QuadroMACS separator (Miltenyi 130-090-976) using the recommend protocol. Briefly, 500 μL of cells were added to a pre-washed column, washed with 3 mL of sorting buffer three times, removed from the separator and washed with a final volume of 5 mL. The final cell fraction from different columns were pooled. Successful PE enrichment was verified by live cell flow cytometry on a Attune NxT (Invitrogen) flow cytometer, showing enrichment of 70%+ in a typical experiment. An example purification result is shown in FIG. 1. Although this method was not used in the results presented in the paper, a second pass on an LS column will yield enrichment up to 90% CD49a+ cells (with downstream resulting SC-beta fractions of >90%), but will decrease recovered cell number. The enriched cells were diluted in Stage 6 media at a concentration of 0.5 M cells per mL and seeded on ultra-low attachment 6-well plates (Corning #3471) with 2 mL of culture per well, placed on a rocker at 27 rpm. to carry out re-aggregation. Clusters were then fed every 48 hours according to the normal protocol. Re-aggregation controls was carried out in rockers for reasons of scale, although it is noted that endocrine enrichment is less efficient than in spinner flasks. Typical yields were approximately 10-15M purified cells when starting with ˜150M total cells. Cells were assessed for function 7-9 days post-purification.

### Preparation of Differentiated Cells for Sequencing

Differentiated clusters were prepared for single cell RNA sequencing as follows: 1-2 mL suspension culture was sampled from the spinner flask, dissociated with TrypLE Express (5-15 minutes at 37° C.), quenched with cold PBS+1% BSA and gently dispersed with a P1000 pipette. Cells were then centrifuged (300 rpm, 3 min), resuspended in cold PBS+1% BSA and filtered through a 70 μm mesh filter. Centrifugation, resuspension and filtering was repeated a total of 3 times. Cells were then counted and resuspended to the working dilution for inDrops (100,000 cells/mL) in 1×PBS with 13% Optiprep (Sigma; D1556).

inDrops Single Cell RNA Sequencing

Single cell RNA sequencing was carried out using the inDrops platform, as previously described^{4,40}. Most samples were run using ‘inDrops v2’ barcoded hydrogel beads (1 Cell Bio, Harvard Single Cell Core), and one experiment used ‘inDrops v3’ beads (Harvard Single Cell Core). Following the inDrops protocol, each biological sample was split into several aliquots of 1000-3000 cells after encapsulation. At least two library aliquots were prepared separately from each sample, indexed using recommended index sequences, pooled and sequenced on a NextSeq 500 (Illumina). The first set of experiments (Stages 3-6 timecourse) involved sequencing several thousand cells per timepoint and provided an estimate of the expected cell type diversity. For the following Stage 5 and 6 time courses, separate flasks were used as technical replicates and measured thousands of cells from each individual timepoint, increasing the capacity for identifying rare populations or subtle changes in the major cell types.

inDrops Raw Data Processing

Sequencing reads were processed according to the previously published inDrops pipeline (github.com/indrops/indrops/). To run the pipeline, a reference index was built from the Ensembl GRCh38 human genome assembly and the GRCh38.88 transcriptome annotation. Briefly, the pipeline trims reads using Trimmomatic, uses Bowtie 1.1.1 to map reads to the human transcriptome, and quantifies transcript expression counts using the unique molecular identifiers, referred to as UMIFMs. For each library, the UMIFM counts matrix was filtered as follows: genes with less than 3 counts were removed; mitochondrially encoded and under-annotated genes were removed; cells with less than 750 (Stage 5 and 6 time courses) or 1000 (all other datasets) UMIFM counts were removed. Variation in the total counts of each individual cell was removed by normalizing the sum of counts of each cell to 10,000. These normalized counts were used as input below and were converted to TPM values for data presentation.

### Dimensionality Reduction and Clustering

Dimensionality reduction and clustering for each dataset was performed by broadly following a modified version of the approach presented in Zeisel et al. 2018⁴¹. Using the unnormalized counts, highly variable genes were identified as previously described⁴¹, by finding outliers with high coefficients of variations as a function of mean expression. Then, within each dataset, (depth normalized) counts values were further z-normalized per gene to yield z-norm values. The z-norm values of variable genes (per dataset) were used as input for principal component analysis (PCA). When computing principal components for the Stage 5 datasets, genes correlated with cell-cycle marker TOP2A (Pearson correlation greater 0.15) were identified and excluded. Clustering was carried out using Leiden community detection⁴², a recently published improvement on Louvain community detection. For community detection, a mutual kNN graph was created by keeping only the mutual edges of the 250 (Stages 5 and 6 time course) or 100 (other datasets) nearest neighbors of cells in the space of the first 50 PCs. When necessary, community detection was repeated on a subset of the cells to improve the cell annotations. It is noted that keeping only mutual edges improved the ability to resolve SST+/HHEX+cells, which correspond to cluster the most difficult to correctly distinguish in the data. For each dataset, this dimensionality reduction procedure followed by clustering was carried out twice per dataset. A first pass was used to identify clusters with lower average library sizes, lack of expression markers (as defined using the score in Zeisel et al.) or clear doublet expression patterns. For the Stage 5 and 6 time course, this first pass of filtering was carried out once per time point, and once again for the complete datasets (with the full datasets used thereafter). The filtered cells were ignored in the second pass of clustering. After this second pass of clustering, individual clusters were assigned an identity (and where appropriate, merged with others) by correlating their expression profiles to a set of predefined marker genes for each population. After clusters were interpreted, a scikit-learn random forest classifier of the clusters was trained and used out-of-bootstrap predictions to assign final labels to the cells. This classifier was also used to recover cells removed in the first pass filter, by retaining cells whose predicted label had a 66% majority across random trees, recovering approximately −5% of the cells across datasets. These retained cells were incorporated in downstream analyses but ignored when finding principal components. tSNE projections were computed with the Python wrapper of the C Barnes-Hut t-SNE implementation (github.com/lvdmaaten/bhtsne), using the first 25 principal components. To compute mean gene expression levels within a label, UMIFM counts were summed for all cells assigned to that label and tpm normalization was computed on these summed counts. The fraction of cells expressing a given gene within a cluster was also computed, using 1% of the maximal expression of that gene (in any cell of the same dataset) as a threshold for qualifying as expressed. The correlation of groups of cells was computed by first selecting 2000 highly variables across the whole dataset, computing the mean expression within each group of cells (as above), z-normalizing each gene across the different classes and then computing Pearson r correlation coefficients between the samples for these 2000 genes.

### Diffusion Pseudotime Analysis

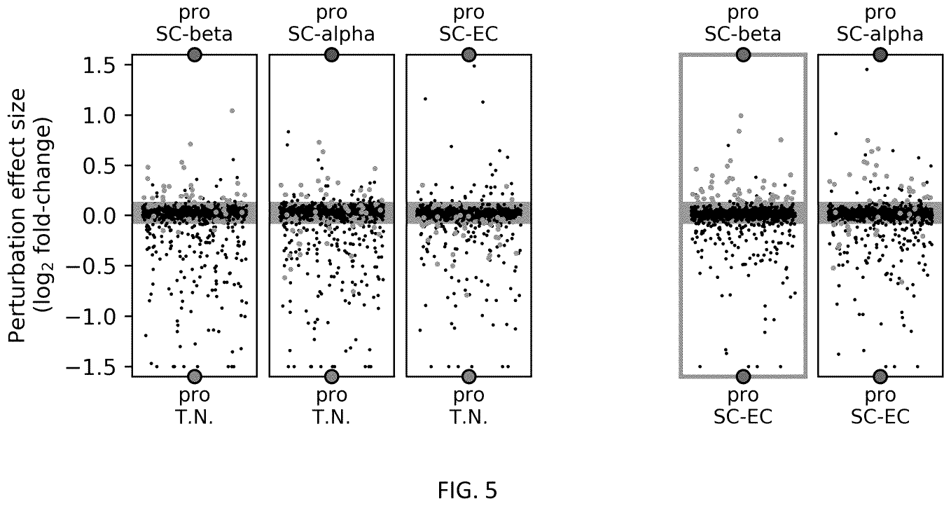

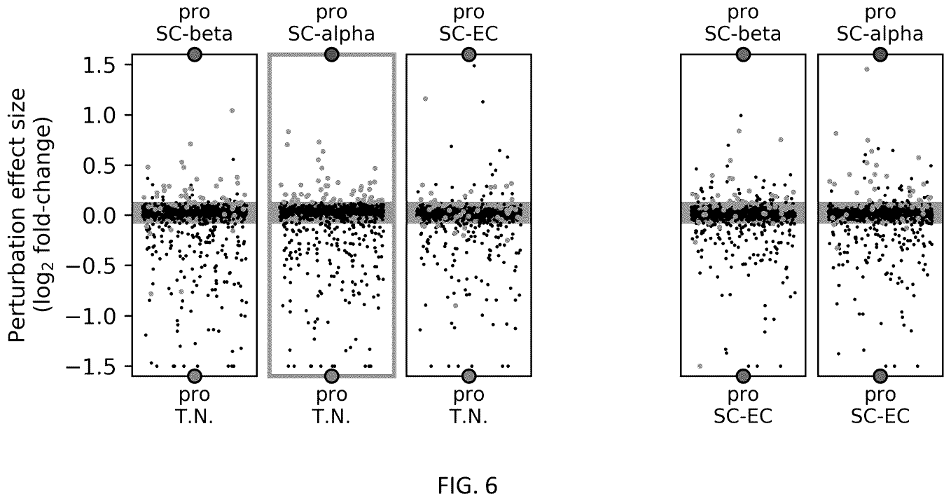

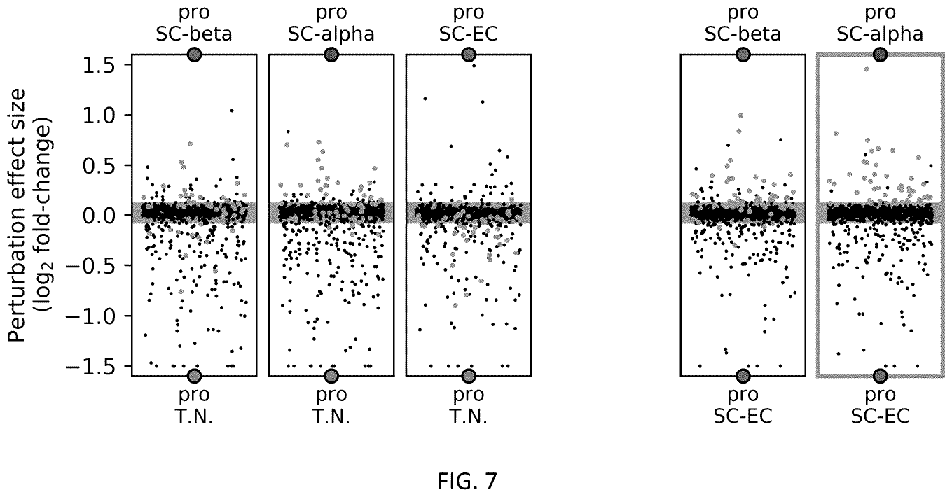

Diffusion pseudotime analysis (DPT)⁴³ was performed using the Scanpy package44, using 100 nearest-neighbors in 10 unscaled principal components to find 10 diffusion components. The DPT was then computed from a manually specified root cell and cells were ordered by their rank along DPT branches (if any). In the Stage 5 branching analysis, cells assigned to the SC-beta or SC-EC clusters were assigned to that branch, while progenitor cells were randomly assigned to a branch. Pseudotime along each branch scales from 0 to 1 corresponding to ranked ordering of the cells, but adjusting the rank of the progenitors such that both branches diverge from the common progenitors at a value of 0.5.To identify genes whose expression is a function of pseudotime, a version of the BEAM⁴⁵ model was implemented. For unbranched pseudotime trajectories, two negative binomial generalized linear models are fit using the VGAM R package. The first is a complete model incorporating a natural spline function of pseudotime. The second is a reduced model which does not include the pseudotime spline term. For branched trajectories, a second complete model incorporates the branch term for each cell as a regression variable. Fold-changes between branches, or across the pseudotime trajectories are then computed using the regressed values. Each regression is run on all the cells being analyzed in that specific analysis, the resulting sample sizes for the regressions are: 10,034 (# of SC-beta cells) for the analysis in FIGS. 11G-11I, 5,131 (# of progenitors at Stage 5, day 0) and 5,109 (# of progenitors at Stage 5, day 1) for the analyses in FIGS. 22C-22E and 18,099 (# of progenitors, endocrine induction, SC-EC or SC-beta cells) for the analysis in FIGS. 14E-14G. As done in the BEAM publication, the likelihood of the data under the complete and reduced models is compared using a likelihood ratio test (with 3 degrees of freedom) and reported as an FDR (alpha=0.001) corrected q-value. It is noted that although this provides a useful relative measure of significance, the significance level is likely inflated because this analysis does not account for the fact that pseudotime values of cells were derived from some of the genes tested in the first place⁴⁶. When reporting fold-changes derived from the pseudotime analysis, a floor on predicted expression (tpm=10) is enforced to prevent artificially high fold-changes. Then, fold-changes between the start and end of the trajectories are calculated by comparing the mean predicted expression in the first and last 5% of the trajectory.

Analysis of Human Pancreatic Islet inDrops Data

Raw sequencing reads from Baron et al.⁵ were reprocessed as described above, to align them the same reference as the in vitro sequencing data. UMIFM counts were converted to tpm for expression analyses as above. Finally, clustering was carried out as described above to identify the same classes of cells as in the original publication.

### Re-Analysis of Beta-Cell EED2 Knockout Data

Processed RNA sequencing data was downloaded from GEO (accession number GSE110648). The read count values were used as input to create linear models using Voom⁴⁷ and Limma⁴⁸. The original data contains three different genotypes (WT, heterozygous and homozygous EED2-floxed alleles) analyzed at two time points (8 and 25 weeks after induction of knock-out). All conditions have triplicate samples, except the heterozygous and homozygous samples at 25 weeks which have duplicates, for a total of 15 samples. A design-contrast parameterization was used to first define replicate groups across all 6 conditions in the dataset and to subsequently identify genes that are differentially expressed between the 25 weeks post-EED2 KO condition for WT, heterozygous and homozygous EED2-floxed alleles. The Benjamini-Hochberg FDR procedure with alpha=0.05 was used to correct for multiple hypothesis testing.

### Re-Analysis of Sorted NKX6.1(GFP)+/−Populations

Complete statistical analyses from Gupta et al.²⁸ were downloaded from the supplementary materials of the publications. The reported mean expression, fold-change and significance values were used directly to generate the relevant figures.

### Gene Set Enrichment Analysis

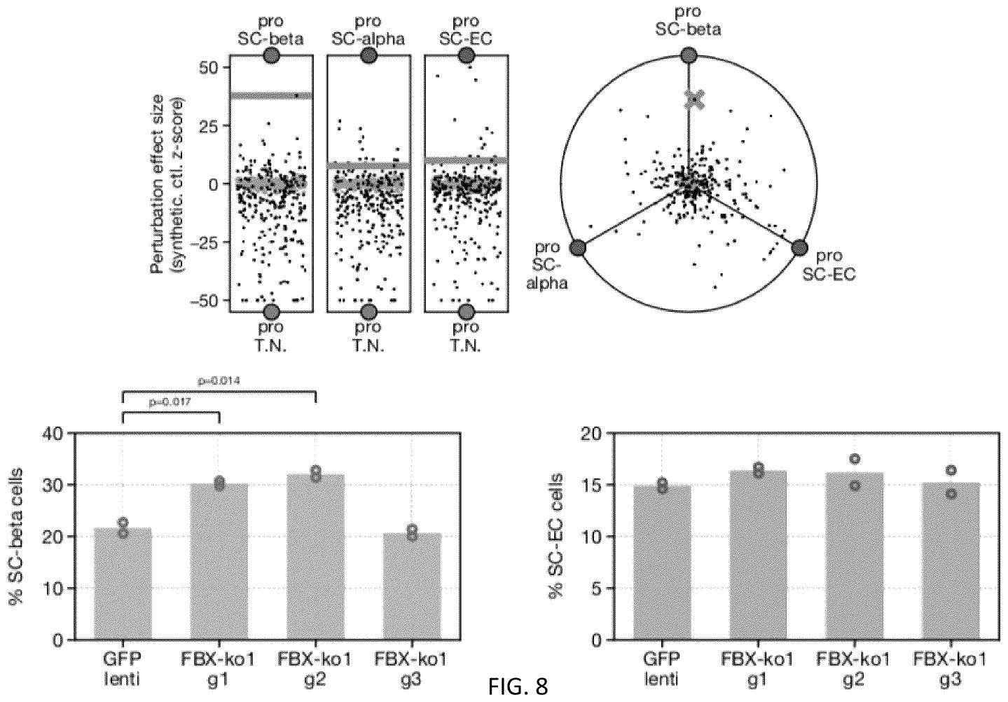

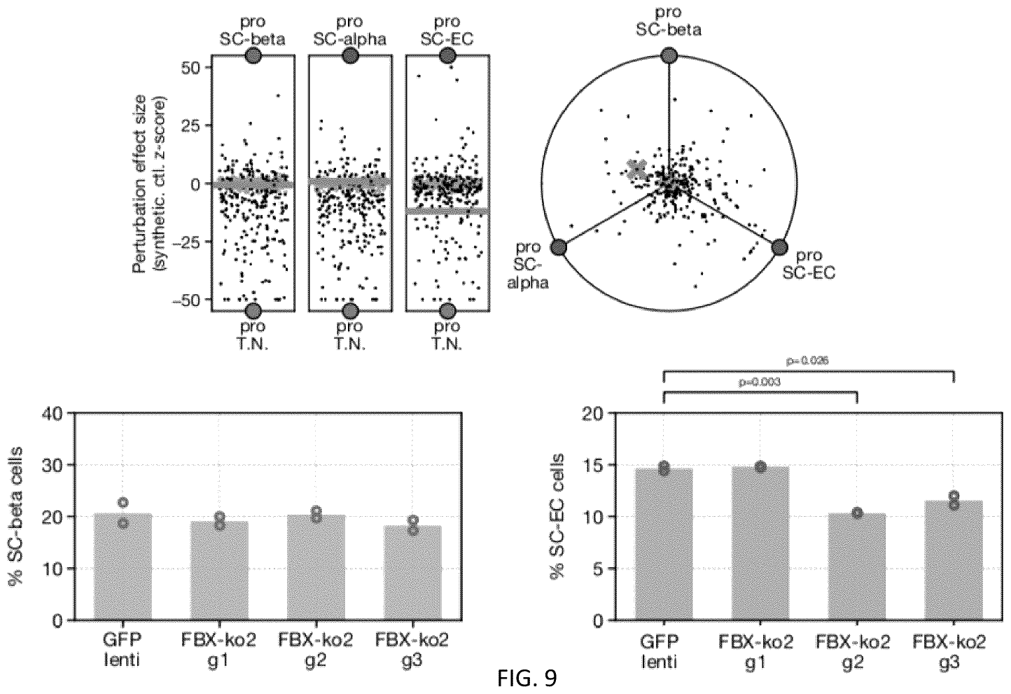

Gene set enrichment analysis (GSEA) was performed using GSEA 3.0 to carry out ‘pre-ranked’ analyses using as input the fold-change between NKX6.1+ progenitors, SC-beta cells and islet beta cells, or the fold-change tracking SC-beta pseudotime expression. The analysis was run including the Hallmark (h.all.v6.2) and Canonical Pathway categories (c2.cp.v6.2) from MSigDB, as well as the custom gene sets defined in FIG. 8 in one single analysis, to ensure appropriate correction for multiple hypothesis testing. Set sizes as small as 5 genes were included, but otherwise run using the default settings.

### REFERENCES

1 Pagliuca, F. W. et al. Generation of functional human pancreatic β cells in vitro. Cell 159, 428-439, doi:10.1016/j.cell.2014.09.040 (2014).
2 Rezania, A. et al. Reversal of diabetes with insulin-producing cells derived in vitro from human pluripotent stem cells. Nat. Biotechnol. 32, 1121-1133, doi:10.1038/nbt.3033 (2014).
3 Russ, H. A. et al. Controlled induction of human pancreatic progenitors produces functional beta-like cells in vitro. EMBO J., e201591058 (2015).
4 Klein, A. M. et al. Droplet barcoding for single-cell transcriptomics applied to embryonic stem cells. Cell 161, 1187-1201, doi:10.1016/j.cell.2015.04.044 (2015).
Baron, M. et al. A Single-Cell Transcriptomic Map of the Human and Mouse Pancreas Reveals Inter- and Intra-cell Population Structure. Cell Syst 3, 346-360.e344, doi:10.1016/j.cels.2016.08.011 (2016).
6 Segerstolpe, Å. et al. Single-Cell Transcriptome Profiling of Human Pancreatic Islets in Health and Type 2 Diabetes. Cell Metab. 24, 593-607, doi:10.1016/j.cmet.2016.08.020 (2016).
7 Xin, Y. et al. RNA Sequencing of Single Human Islet Cells Reveals Type 2 Diabetes Genes. Cell Metab. 24, 608-615, doi:10.1016/j.cmet.2016.08.018 (2016).
8 Muraro, M. J. et al. A Single-Cell Transcriptome Atlas of the Human Pancreas. Cell Syst 3, 385-394.e383, doi:10.1016/j.cels.2016.09.002 (2016).
9 Enge, M. et al. Single-Cell Analysis of Human Pancreas Reveals Transcriptional Signatures of Aging and Somatic Mutation Patterns. Cell 171, 321-330.e314, doi:10.1016/j.cell.2017.09.004 (2017).
10 Byrnes, L. E. et al. Lineage dynamics of murine pancreatic development at single-cell resolution. Nature Communications 9, 3922, doi:10.1038/s41467-018-06176-3 (2018).
11 Scavuzzo, M. A. et al. Endocrine lineage biases arise in temporally distinct endocrine progenitors during pancreatic morphogenesis. Nature Communications 9, 3356, doi:10.1038/s41467-018-05740-1 (2018).
12 Xie, R. et al. Dynamic chromatin remodeling mediated by polycomb proteins orchestrates pancreatic differentiation of human embryonic stem cells. Cell Stem Cell 12, 224-237, doi:10.1016/j.stem.2012.11.023 (2013).
13 Hrvatin, S. et al. Differentiated human stem cells resemble fetal, not adult, R cells. Proc. Natl. Acad. Sci. U.S.A 111, 3038-3043, doi:10.1073/pnas.1400709111 (2014).
14 Petersen, M. B. K. et al. Single-Cell Gene Expression Analysis of a Human ESC Model of Pancreatic Endocrine Development Reveals Different Paths to β-Cell Differentiation. Stem Cell Reports 9, 1246-1261, doi:10.1016/j.stemcr.2017.08.009 (2017).
15 Rutter, G. A., Pullen, T. J., Hodson, D. J. & Martinez-Sanchez, A. Pancreatic beta-cell identity, glucose sensing and the control of insulin secretion. (2015).
16 Thurmond, D. C. in Mechanisms of Insulin Action 52-70 (Springer, 2007).
17 Aslamy, A. & Thurmond, D. C. Exocytosis proteins as novel targets for diabetes prevention and/or remediation? (2017).
18 Arda, H. E. et al. Age-Dependent Pancreatic Gene Regulation Reveals Mechanisms Governing Human β Cell Function. Cell Metab. 23, 909-920, doi:10.1016/j.cmet.2016.04.002 (2016).
19 Blum, B. et al. Functional beta-cell maturation is marked by an increased glucose threshold and by expression of urocortin 3. Nat. Biotechnol. 30, 261-264, doi:10.1038/nbt.2141 (2012).
20 Thorrez, L. et al. Tissue-specific disallowance of housekeeping genes: the other face of cell differentiation. Genome Res. 21, 95-105, doi:10.1101/gr.109173.110 (2011).
21 Kelly, O. G. et al. Cell-surface markers for the isolation of pancreatic cell types derived from human embryonic stem cells. Nature Biotechnology 29, 750, doi:10.1038/nbt.1931 (2011).
22 Riedel, M. J. et al. Immunohistochemical characterisation of cells co-producing insulin and glucagon in the developing human pancreas. Diabetologia 55, 372-381, doi:10.1007/s00125-011-2344-9 (2012).
23 Spijker, H. S. et al. Loss of β-Cell Identity Occurs in Type 2 Diabetes and Is Associated With Islet Amyloid Deposits. Diabetes 64, 2928, doi:10.2337/dbl4-1752 (2015).
24 Bellono, N. W. et al. Enterochromaffin Cells Are Gut Chemosensors that Couple to Sensory Neural Pathways. Cell 170, 185-198.e116, doi:10.1016/j.cell.2017.05.034 (2017).
25 Haber, A. L. et al. A single-cell survey of the small intestinal epithelium. Nature 551, 333-339, doi:10.1038/nature24489 (2017).
26 Grün, D. et al. Single-cell messenger RNA sequencing reveals rare intestinal cell types. Nature 525, 251-255, doi:10.1038/nature14966 (2015).
27 Martin, A. M. et al. The nutrient-sensing repertoires of mouse enterochromaffin cells differ between duodenum and colon. Neurogastroenterol. Motil. 29, doi:10.1111/nmo.13046 (2017).
28 Gupta, S. K. et al. NKX6.1 induced pluripotent stem cell reporter lines for isolation and analysis of functionally relevant neuronal and pancreas populations. Stem Cell Research 29, 220-231, doi:10.1016/j.scr.2018.04.010 (2018).
29 Almaga, J. et al. Human Beta Cells Produce and Release Serotonin to Inhibit Glucagon Secretion from Alpha Cells. Cell Rep. 17, 3281-3291, doi:10.1016/j.celrep.2016.11.072 (2016).
30 Goyvaerts, L., Schraenen, A. & Schuit, F. Serotonin competence of mouse beta cells during pregnancy. Diabetologia 59, 1356-1363, doi:10.1007/s00125-016-3951-2 (2016).
31 Ohta, Y. et al. Convergence of the insulin and serotonin programs in the pancreatic β-cell. Diabetes 60, 3208-3216, doi:10.2337/db10-1192 (2011).
32 Lu, T. T.-H. et al. The Polycomb-Dependent Epigenome Controls β Cell Dysfunction, Dedifferentiation, and Diabetes. Cell Metabolism 27, 1294-1308.e1297, doi:10.1016/j.cmet.2018.04.013 (2018).
33 Britt, L. D., Stojeba, P. C., Scharp, C. R., Greider, M. H. & Scharp, D. W. Neonatal pig pseudo-islets. A product of selective aggregation. Diabetes 30, 580-583 (1981).
34 Agulnick, A. D. et al. Insulin-producing endocrine cells differentiated in vitro from human embryonic stem cells function in macroencapsulation devices in vivo. Stem Cells Transl. Med. 4, 1214-1222 (2015).
35 Tsoukalas, N. et al. Pancreatic carcinoids (serotonin-producing pancreatic neuroendocrine neoplasms): Report of 5 cases and review of the literature. Medicine 96, e6201, doi:10.1097/MD.0000000000006201 (2017).
36 Hilderink, J. et al. Controlled aggregation of primary human pancreatic islet cells leads to glucose-responsive pseudoislets comparable to native islets. J. Cell. Mol. Med. 19, 1836-1846 (2015).
37 Ramachandran, K., Peng, X., Bokvist, K. & Stehno-Bittel, L. Assessment of re-aggregated human pancreatic islets for secondary drug screening. Br. J. Pharmacol. 171, 3010-3022 (2014).
38 Spijker, H. S. et al. Conversion of mature human 3-cells into glucagon-producing a-cells. Diabetes 62, 2471-2480, doi:10.2337/db12-1001 (2013).
39 Zuellig, R. A. et al. Improved physiological properties of gravity-enforced reassembled rat and human pancreatic pseudo-islets. J. Tissue Eng. Regen. Med. 11, 109-120 (2017).
40 Zilionis, R. et al. Single-cell barcoding and sequencing using droplet microfluidics. Nat. Protoc. 12, 44-73, doi:10.1038/nprot.2016.154 (2017).
41 Zeisel, A. et al. Molecular Architecture of the Mouse Nervous System. Cell 174, 999-1014.e1022, doi:10.1016/j.cell.2018.06.021 (2018).
42 Traag, V., Waltman, L. & Eck, N. J. v. From Louvain to Leiden: guaranteeing well-connected communities. arXiv (2018).
43 Haghverdi, L., BUttner, M., Wolf, F. A., Buettner, F. & Theis, F. J. Diffusion pseudotime robustly reconstructs lineage branching. Nat. Methods 13, 845-848, doi:10.1038/nmeth.3971 (2016).
44 Wolf, F. A., Angerer, P. & Theis, F. J. SCANPY: large-scale single-cell gene expression data analysis. Genome Biol. 19, 15, doi:10.1186/s13059-017-1382-0 (2018).
45 Qiu, X. et al. Single-cell mRNA quantification and differential analysis with Census. Nature Methods 14, 309, doi:10.1038/nmeth.4150 (2017).
46 Zhang, J. M., Kamath, G. M. & Tse, D. N. Towards a post-clustering test for differential expression. bioRxiv, 463265, doi:10.1101/463265 (2018).
47 Law, C. W., Chen, Y., Shi, W. & Smyth, G. K. voom: Precision weights unlock linear model analysis tools for RNA-seq read counts. Genome Biol. 15, R29, doi:10.1186/gb-2014-15-2-r29 (2014).
48 Smyth, G. K. Linear models and empirical bayes methods for assessing differential expression in microarray experiments. Stat. Appl. Genet. Mol. Biol. 3, Article3, doi:10.2202/1544-6115.1027 (2004).

1. A method for directing differentiation of a population of cells comprising inhibiting expression of a regulator of cell fate in a progenitor cell, wherein the regulator is a gene controlling cell fate, thereby directing differentiation of a population of cells toward SC-β cells.

2. The method of claim 1, wherein the regulator is selected from the group consisting of FBXL14, BCORL1, SHOC2, CCDC6, B3GALT6, HOXA1, DDX3X, CARM1, EXT2, EXT1, DYRK1A, SCAF1, SCAF8, CAND1, NDST1, EYA3, GLCE, DYRK1B, PRDM16, ALG3, CXXC4, SMURF1, PHF21A, SOX4, and TET2.

3. (canceled)

4. The method of claim 1, wherein the regulator is selected from the group consisting of SOX4, BCORL1, FBXL14, CCDC6, SOX1, CARM1, TNRC18, CAND1, TET2, HOXA1, ASCL1, ARID2, SIRT6, FBXO22, FLVCR1, FOXA1, COPS9, ELAVL1, SSBP3, PROSER1, PROX1, SMURF1, SCAF1, HELLS, and DACH1.

5. (canceled)

6. The method of claim 1, wherein the expression of the regulator of cell fate is inhibited by knocking down the regulator using a gene editing technique.

7. The method of claim 6, wherein the expression of the regulator of cell fate is inhibited by knocking down the regulating using CRISPR.

8. The method of claim 1, wherein the expression of the regulator of cell fate is inhibited by knocking out the regulator using a gene editing technique.

9. The method of claim 8, wherein the expression of the regulator of cell fate is inhibited by knocking out the regulator using CRISPR.

10. (canceled)

11. (canceled)

12. An enriched population of SC-β cells produced by the method of claim 1.

13. (canceled)

14. (canceled)

15. An SC-islet comprising the enriched population of SC-β cells of claim 12.

16. A method for directing differentiation of a population of cells comprising inhibiting expression of a regulator of cell fate in a progenitor cell, wherein the regulator is a gene controlling cell fate, thereby directing differentiation of a population of cells towards SC-α cells.

17. The method of claim 16, wherein the regulator is selected from the group consisting of PDX1, CCDC6, HES1, PHF21A, PAX4, DYRK1B, DYRK1A, BCORL1, TET2, DDX3X, PROSER1, PBX1, HELLS, CAND1, EYA3, MYT1, AFF4, FBXL14, HOXA1, ZC3H15, SCAF8, PRDM16, HEXIM1, TTC14, ZRANB1, and B3GALT6.

18. (canceled)

19. The method of claim 16, wherein the regulator is selected from the group consisting of PAX4, HES1, CCDC6, SOX4, ZBTB10, PHF21A, PBX1, ARID2, TET2, BCORL1, TTC14, CAND1, PROSER1, SOX1, FBXO22, HELLS, DYRK1B, ZRANB1, DYRK1A, ASCL1, ZC3H15, SETBP1, FAM58A, MYT1, and RALGAPB.

20. (canceled)

21. The method of claim 16, wherein the expression of the regulator of cell fate is inhibited by knocking down the regulator using a gene editing technique.

22. The method of claim 21, wherein the expression of the regulator of cell fate is inhibited by knocking down the regulating using CRISPR.

23.-26. (canceled)

27. An enriched population of SC-α cells produced by the method of claim 16.

28.-30. (canceled)

31. A cell that has been modified to have reduced expression of one or more of the following genes: FBXL14, BCORL1, SHOC2, CCDC6, B3GALT6, HOXA1, DDX3X, CARM1, EXT2, EXT1, DYRK1A, SCAF1, SCAF8, CAND1, NDST1, EYA3, GLCE, DYRK1B, PRDM16, ALG3, CXXC4, SMURF1, PHF21A, SOX4, TET2, SOX4, BCORL1, FBXL14, CCDC6, SOX1, CARM1, TNRC18, CAND1, TET2, HOXA1, ASCL1, ARID2, SIRT6, FBXO22, FLVCR1, FOXA1, COPS9, ELAVL1, SSBP3, PROSER1, PROX1, SMURF1, SCAF1, HELLS, or DACH1.

32. The cell of claim 31, wherein the expression of the one or more genes is knocked-down.

33. The cell of claim 31, wherein the one or more genes is knocked-out.

34. The cell of claim 31, wherein the cell is a stem cell.

35. The cell of claim 31, wherein the cell is a SC-β cell.

36. A composition comprising the cell of claim 31 and a pharmaceutically acceptable carrier.

37. A method of treating a subject with diabetes, comprising administering to the subject the composition of claim 36.

38. A cell that has been modified to have reduced expression of one or more of the following genes: PDX1, CCDC6, HES1, PHF21A, PAX4, DYRK1B, DYRK1A, BCORL1, TET2, DDX3X, PROSER1, PBX1, HELLS, CAND1, EYA3, MYT1, AFF4, FBXL14, HOXA1, ZC3H15, SCAF8, PRDM16, HEXIM1, TTC14, ZRANB1, B3GALT6, PAX4, HES1, CCDC6, SOX4, ZBTB10, PHF21A, PBX1, ARID2, TET2, BCORL1, TTC14, CAND1, PROSER1, SOX1, FBXO22, HELLS, DYRK1B, ZRANB1, DYRK1A, ASCL1, ZC3H15, SETBP1, FAM58A, MYT1, and RALGAPB.

39. The cell of claim 38, wherein the expression of the one or more genes is knocked-down.

40. The cell of claim 38, wherein the one or more genes is knocked-out.

41. The cell of claim 38, wherein the cell is a stem cell.

42. The cell of claim 38, wherein the cell is a SC-α cell.

43. A composition comprising the cell of claim 38 and a pharmaceutically acceptable carrier.

44. A method of treating a subject with diabetes, comprising administering to the subject the composition of claim 43.
